(19) United States
(12) Patent Application Publication
(10) Pub. No.: US 2026/0354759 A1
(43) Pub. Date: Nov. 12, 2026

(54) MODULAR BIOSIGNAL ACQUISITION PLATFORM WITH STANDARDIZED ELECTROMECHANICAL INTERFACE, SIGNAL-CONDUCTING TENSIONING ELEMENT, AND ADAPTIVE TOPOLOGY-AWARE SIGNAL PROCESSING

(71) Applicant: Cognixion Corporation, Santa Barbara, CA (US)

(72) Inventors: Joseph Andreas Forsland, Santa Barbara, CA (US); Christopher Jason Ullrich, Ventura, CA (US); Rostyslav Matiukhin, Kiev (UA)

(73) Assignee: Cognixion Corporation, Santa Barbara, CA (US)

(21) Appl. No.: 19/672,417

(22) Filed: May 8, 2026

Related U.S. Application Data
(60) Provisional application No. 63/802,382, filed on May 8, 2025.

(51) Int. Cl. A61B 5/00

(52) U.S. Cl. CPC A61B 5/6803; A61B 2560/0285; A61B 2560/0443; A61B 2562/182; A61B 2562/225

(57) ABSTRACT

A modular head-mounted biosignal acquisition platform, a method for use, an accessory interface specification, and a non-transitory computer-readable medium are disclosed. The platform comprises a wearable structural chassis with a standardized electromechanical mating interface that defines physical connector geometry, mechanical retention, electrical pin assignment (power, ground, biosignal channel, digital identification) and a module-identification signaling protocol so that any conforming sensor module (e.g., EEG, fNIRS, ultrasound, EMG, electrical stimulation) can be physically and electrically attached. A processing system detects and identifies connected modules from the interface, builds a sensor configuration, computes per-channel confidence measures (e.g., impedance, signal variance, power-line interference, contact quality) and adjusts channel weights in the signal-processing pipeline to improve SNR. A wearable strap assembly integrates tensioning elements with electrical conductors in a composite structure to simultaneously provide mechanical fit and conduct biosignal or power.

### CROSS-REFERENCE TO RELATED APPLICATIONS

This application claims the benefit of U.S. provisional patent application Ser. No. 63/802,382, filed on May 8, 2025, the contents of which are incorporated herein by reference in their entirety.

### TECHNICAL FIELD

Sensor-agnostic biosignal acquisition platform, demonstrated with electroencephalography (EEG) and illustrative of functional near-infrared spectroscopy (fNIRS), transcranial ultrasound, electromyography (EMG), and transcranial electrical/magnetic stimulation modalities. The disclosure further relates to modular head-mounted device architectures, standardized electromechanical accessory interfaces, wearable computing, augmented reality systems, and adaptive signal-processing pipelines for brain-computer interfaces.

### BACKGROUND

Conventional head-mounted biosignal acquisition systems are closed, single-modality, and non-extensible. Biosensing electrodes and structural chassis are permanently integrated in conventional solutions, preventing sterilization, replacement, or upgrade of individual components without discarding the entire device. Strap adjustment mechanisms are purely mechanical, requiring separate wiring harnesses to route biosensor signals. This adds weight, failure points, and assembly complexity. There is no standardized, published, hardware-level interface permitting third-party manufacturers to produce compatible biosensing modules or peripherals for head-mounted biosignal acquisition systems.

Conventional and currently available acquisition systems lack automatic configuration: when sensor topology changes (module added, removed, or reconfigured), users must manually intervene in firmware or software. Current systems cannot alternate or combine multiple sensing and stimulation modalities (e.g., EEG, fNIRS, ultrasound, transcranial electrical stimulation or tES, transcranial magnetic stimulation or TMS, etc.) in a unified modular form factor. Closed-loop neurofeedback systems such as those available with conventional solutions do not leverage environmental context (e.g., data from cameras, microphones, ambient sensors) fused with biosignal data in a unified platform.

There is, therefore, a need for a head-mounted biosignal acquisition device and system that provides lighter wearable components and more flexible hardware and software integrations to better support a broad array of applications.

### BRIEF SUMMARY

A head-mounted biosignal acquisition platform includes a structural chassis configured to be worn on a user's head and a standardized electromechanical mating interface mounted on that chassis. The standardized interface specifies a physical connector geometry, a mechanical retention specification, an electrical pin assignment that includes at least one power conductor, at least one ground conductor, at least one biosignal channel conductor, and at least one digital identification conductor, and a module-identification signaling protocol. At least one sensor module is coupled to the structural chassis via this standardized interface, and the sensor module is configured to acquire a biosignal selected from electroencephalography (EEG), functional near-infrared spectroscopy (fNIRS), ultrasound, electromyography (EMG), and electrical stimulation signals. The standardized interface is defined by the listed physical, mechanical, electrical and signaling elements so that the structural chassis is configured to physically and electrically engage any sensor module that conforms to all of those elements.

A method of operating the modular biosignal acquisition platform comprises, by a processing system coupled to the structural chassis, detecting one or more sensor modules connected to the chassis via the standardized electromechanical mating interface; identifying, from a module-identification signal received via that interface, a module type and a capability profile for each detected sensor module; constructing a sensor configuration representation based on the identified sensor modules; computing, for each channel of each detected sensor module, a confidence measure based on at least one of electrical impedance, signal variance, power-line interference, and a contact-quality metric; and adjusting a weight assigned to each channel in a signal-processing pipeline based on the computed confidence measure.

A wearable biosignal acquisition strap assembly comprises a first strap segment and a second strap segment configured to be worn on a user's head, and at least one tensioning element disposed along at least one of the strap segments and configured to apply mechanical tension to maintain fit on the user's head. At least one electrical conductor is integrated with the tensioning element, and the tensioning element together with the electrical conductor form a composite structure that, during operation, concurrently applies mechanical tension and conducts at least one of a biosignal and a power signal between a sensor module and a processing system.

An accessory interface specification for a head-mounted device defines a physical connector geometry, a mechanical retention specification, an electrical pin assignment that includes at least one power conductor, at least one ground conductor, at least one biosignal channel conductor, and at least one digital identification conductor, and a module-identification signaling protocol. The accessory interface specification is configured so that any module conforming to those defined physical, mechanical, electrical and signaling elements may be physically and electrically engaged with a head-mounted device implementing the specification without modification of the head-mounted device. The module-identification signaling protocol is configured to identify a module type and a capability profile of the conforming module to the processing system of the head-mounted device.

A non-transitory computer-readable medium stores instructions that, when executed by a processing system coupled to the structural chassis of a head-mounted device, cause the processing system to detect one or more sensor modules connected via the standardized electromechanical mating interface; identify, from a module-identification signal received via that interface, a module type and a capability profile for each detected sensor module; construct a sensor configuration representation based on the identified sensor modules; compute, for each channel of each detected sensor module, a confidence measure based on at least one of electrical impedance, signal variance, power-line interference, and a contact-quality metric; and adjust a weight assigned to each channel in a signal-processing pipeline based on the computed confidence measure, thereby improving signal-to-noise ratio of biosignals acquired by the head-mounted device.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an exemplary head-mounted biosignal acquisition platform 100 in accordance with one embodiment.

FIG. 2A-2E illustrates a modular head-mounted biosignal acquisition system 200 in accordance with one embodiment.

FIG. 3A-3C illustrates a structural chassis assembly 300 in accordance with one embodiment.

FIG. 4 illustrates a wearable biosignal acquisition strap assembly 400 in accordance with one embodiment.

FIG. 5 illustrates an exemplary standardized electromechanical mating interface 500 in accordance with one embodiment.

FIG. 6A-6B illustrates a tensioning element installation and operation 600 in accordance with one embodiment.

FIG. 7 illustrates a strap assembly connector arm details 700 in accordance with one embodiment.

FIG. 8 illustrates a processing system 800 in accordance with one embodiment.

FIG. 9 illustrates an exemplary modular biosignal acquisition platform components 900 in accordance with one embodiment.

FIG. 10 illustrates a routine 1000 in accordance with one embodiment.

FIG. 11 illustrates a user agency and capability augmentation system 1100 in accordance with one embodiment.

FIG. 12 illustrates a biosignals subsystem 1200 in accordance with one embodiment.

FIG. 13 illustrates a context subsystem 1300 in accordance with one embodiment.

FIG. 14 illustrates a routine 1400 in accordance with one embodiment.

FIG. 15 illustrates a multiuser system 1500 in accordance with one embodiment.

FIG. 16 illustrates an exemplary tokenizer 1600 in accordance with one embodiment.

FIG. 17A illustrates an isometric view of a BCI headset system 1700 in accordance with one embodiment.

FIG. 17B illustrates a rear view of a BCI headset system 1700 in accordance with one embodiment.

FIG. 17C and FIG. 17D illustrate exploded views of a BCI headset system 1700 in accordance with one embodiment.

FIG. 18 illustrates a BCI+AR environment 1800 in accordance with one embodiment.

FIG. 19 illustrates an augmented reality device logic 1900 in accordance with one embodiment.

FIG. 20 illustrates a block diagram of nonverbal multi-input and feedback device 2000 in accordance with one embodiment.

FIG. 21 illustrates a block diagram of a single framework of a nonverbal multi-input and feedback device 2100 in accordance with one embodiment.

FIG. 22 illustrates a block diagram of nonverbal multi-input and feedback device 2200 in accordance with one embodiment.

FIG. 23 illustrates a logical diagram of a user wearing an augmented reality headset 2300 in accordance with one embodiment.

FIG. 24 a logical diagram of a user wearing an augmented reality headset 2400 in accordance with one embodiment.

FIG. 25 illustrates a diagram of a use case including a user wearing an augmented reality headset 2500 in accordance with one embodiment.

FIG. 26 illustrates a flow diagram 2600 in accordance with one embodiment.

FIG. 27 illustrates a flow diagram 2700 in accordance with one embodiment.

FIG. 28 illustrates a block diagram 2800 in accordance with one embodiment.

FIG. 29 illustrates a block diagram 2900 in accordance with one embodiment.

FIG. 30 illustrates a block diagram 3000 in accordance with one embodiment.

FIG. 31 illustrates a computing device 3100 in accordance with one embodiment.

FIG. 32 illustrates a cloud computing system 3200 in accordance with one embodiment.

FIG. 33 illustrates cloud computing functional abstraction layers 3300 in accordance with one embodiment.

### DETAILED DESCRIPTION

A sensor-agnostic biosignal acquisition platform is disclosed herein. The platform is organized around a standardized electromechanical mating interface that allows modular sensor cartridges, third-party accessories, and multiple sensing modalities to be connected, detected, and integrated into the signal-processing pipeline at runtime. Modularity is the central principle: structural components, sensor cartridges, strap elements, and external compute modules are independently replaceable, sterilizable, and interchangeable via the standardized interface.

The disclosed platform has a number of principal technical contributions. A published, standardized electromechanical and electrical interface specification defines connector geometry, mechanical retention, electrical pin assignment, and a communication protocol. This interface specification supports an ecosystem of compatible devices and modules, including third-party accessories. Further, an automatic sensor-topology detection and confidence-weighted signal-processing method allows the platform to discover connected modules, construct a sensor graph, compute per-channel confidence, and dynamically re-weight the signal-processing pipeline.

In addition, the platform includes a signal-conducting tensioning element in the head strap. The tensioning element concurrently provides mechanical tension for device fit and functions as an electrical conductor for biosignal routing. This supports elimination of separate wiring harnesses. In some embodiments the tensioning element may be combined with a rolling/rotational adjustment mechanism containing concentric electrical contacts. Finally, a context-aware closed-loop feedback system is implemented in the disclosed platform. This feedback system fuses environmental sensor data with biosignal data using an AI/ML model running locally, remotely, or hybrid. Feedback is returned to the user through visual, audio, or haptic modalities selected based on detected hardware.

In one embodiment, the head-mounted biosignal acquisition platform includes a standardized module interface specification and/or an accessory interface specification. This may be a published interface specification defining a physical connector geometry, including alignment features and retention method. The standardized module interface specification may include an electrical pin assignment covering power, ground, analog biosignal channels, digital communication, and module-identification signaling. The standardized module interface specification may include a mechanical retention specification (magnetic, latching, or hybrid). The standardized module interface specification may include a module-identification protocol by which a connected module reports its type, capability profile, and calibration metadata to the host platform. Third-party accessory manufacturers licensing or conforming to the specification may produce physically and electrically compatible modules without modification to the host chassis. The host platform may be the processing system disclosed herein, and the host chassis may be the structural chassis also disclosed. The interface specification disclosed herein may be claimed, licensed, or distributed as a thing apart from any specific host platform that implements the specification, and a host chassis that implements the standardized electromechanical mating interface may be operated without an installed sensor module so that a user may supply a conforming module from a third-party manufacturer.

The standardized interface may be implemented using at least one magnet paired with at least one electrical contact, where the magnet provides mechanical coupling force and the contact simultaneously provides electrical continuity. The interface may be self-aligning and tool-free. The interface may support rapid assembly, disassembly, and independent component sterilization. Various embodiments may include concentric ring contacts, pogo-pin arrays, and flex-circuit interposers, each mated with permanent or electropermanent magnets.

In one embodiment, the disclosed solution comprises an accessory interface specification for a head-mounted device. The accessory interface specification may include a physical connector geometry, a mechanical retention specification, an electrical pin assignment defining at least one power conductor, at least one ground conductor, at least one biosignal channel conductor or general or other signaling channel conductor, and at least one digital identification conductor, and. The accessory interface specification may also include a module-identification signaling protocol. The accessory interface specification may be configured such that any module conforming to the physical connector geometry, mechanical retention specification, electrical pin assignment, and module-identification signaling protocol is physically and electrically engageable with a head-mounted device implementing the accessory interface specification without modification of the head-mounted device. The module-identification signaling protocol may be configured to identify a module type and a capability profile of the conforming module to a processing system of the head-mounted device.

In one embodiment, the disclosed platform may include interchangeable sensor cartridges conforming to the standardized interface. These may include dry or wet EEG electrode arrays, fNIRS emitter/detector pairs; transcranial ultrasound transducer arrays, transcranial electrical stimulation (tES/tDCS/tACS) electrodes, transcranial magnetic stimulation (TMS) coil elements, and null/comfort cartridges containing no active sensing elements. The platform may be designed to alternate between acquisition and stimulation modes on the same electrode interface where appropriate. Cartridges may be disposable, reusable, or reusable with disposable contact surfaces.

In one embodiment, the standardized electromechanical mating interface may receive a bridging module configured to communicate with an implantable biosignal acquisition device. The bridging module may provide at least one of transcutaneous inductive power and data coupling, near-field radio-frequency communication, percutaneous connector pass-through, and ultrasonic data and power coupling between the implantable device and the head-mounted platform. The bridging module may conform to the physical connector geometry, mechanical retention specification, electrical pin assignment, and module-identification signaling protocol of the standardized interface, such that biosignal data acquired by the implantable device is presented to the host platform as if the implantable device were any other conforming sensor module. In this manner, the disclosed platform may serve as a non-invasive host for invasive or partially invasive biosignal acquisition systems, including, without limitation cortical electrode arrays, endovascular electrode arrays, and subdermal electrode arrays.

In one embodiment, upon power-on or module connection/removal, the platform may query all mating interfaces, read module-identification metadata, and construct a sensor configuration graph. A confidence measure may be computed per sensor channel based on electrode impedance, signal variance, power-line interference, or contact-quality proxies. Channels with low confidence may receive reduced weighting in the signal-processing model. The user may be alerted via visual augmented reality (AR) overlay, audio tone, or haptic cue to reposition or replace low-confidence modules.

In one embodiment, the platform may further support a conformance-testing protocol by which a candidate sensor module supplied by a third-party manufacturer is verified for compatibility with the standardized electromechanical mating interface. The conformance-testing protocol may comprise receiving a module-identification signal from the candidate sensor module via the standardized interface; verifying that the received module-identification signal conforms to a predefined module-identification signaling protocol; verifying that the candidate sensor module is physically and electrically compatible with the connector geometry, mechanical retention specification, and electrical pin assignment of the standardized interface; and producing a certification indicator. The certification indicator may be recorded in non-volatile memory of the candidate sensor module, transmitted to a certification registry, or both. A platform supporting the conformance-testing protocol may permit selective acceptance of conforming modules, exclusion of non-conforming modules, or both.

In one embodiment, sensor modules may comprise interchangeable sensor cartridges including dry or wet electroencephalography electrode arrays, functional near-infrared spectroscopy emitter and detector pairs, transcranial ultrasound transducer arrays, transcranial electrical stimulation electrodes, transcranial magnetic stimulation coil elements, and a bridging module configured to communicate with an implantable biosignal acquisition device.

In one embodiment, one or more tensioning elements may be disposed within the head strap and may simultaneously provide mechanical tension for device fit and function as electrical conductors for biosignal data or power. Implementations may include conductive fibers woven or braided around a tension cable, a conductive stainless-steel wire rope with insulating coating, co-extruded composites, a flex-circuit ribbon integrated with a tensioning tape, or other configurations that will be readily apparent to one of ordinary skill in the art. Conductive elements may support shielding against electromagnetic interference for biosignal fidelity.

In one embodiment, a rotational dial located at the intersection of strap segments may use a rolling motion to adjust tension. The dial may contain concentric conductive rings or brush contacts that maintain electrical continuity across rotational positions, allowing multi-channel biosignal data to pass through the dial. Press-in/pull-out secondary motions may engage independent adjustment modes. Ratcheted detents provide discrete size steps, and a lock feature secures the dial at a selected position.

In one embodiment, outward-facing environmental sensors such as red-green-blue (RGB) cameras, depth sensors, microphones, ambient-light detectors, etc., produce a context data stream. A processing system, executing locally on a body-worn compute module, remotely in a cloud service, or in a hybrid configuration, may fuse the context data with biosignal data via an artificial intelligence (AI)/machine learning (ML) model to compute a user-state estimate and generate feedback. Feedback is rendered through an AR display, audio output, or haptic actuator, with the modality selected based on which feedback-capable modules are currently detected on the platform. In a further embodiment, feedback rendered through the AR display may include rendering of visually evoked stimulus elements, including without limitation frequency-modulated, motion-modulated, or code-modulated shaders, overlaid on or proximate to interactive user-interface elements rendered by an underlying operating system or application, such that user attention to or gaze fixation on a shader-bearing element produces a measurable evoked-potential response detectable via the disclosed platform; the shaders may be applied across multiple applications and across native operating-system user-interface elements such that the disclosed platform provides operating-system-level evoked-potential interaction without modification of the underlying applications, and the shaders may be activated in response to detected gaze, dwell, hover, or proximity of a virtual cursor to a corresponding user-interface element.

In one embodiment, an external body-worn compute module (hip-pack) connects to the head-mounted device via cable or wireless link and provides compute specifically designed for algorithmic workloads such as machine learning inference. The hip-pack exposes the standardized accessory interface to third-party peripherals including keyboards, joystick controllers, expansion sensor arrays, and ML accelerator modules. The hip-pack may optionally include a touch-sensitive status display, speech input, and an electrically isolated trigger input for synchronization with external stimulus systems. The hip-pack may optionally dock directly to the head-mounted device for a self-contained form factor.

In one embodiment, the standardized electromechanical mating interface disclosed herein may carry a logical-layer protocol stack such that the device of the disclosed platform appears to a connected host computing device as a Human Interface Device (HID) over Bluetooth Low Energy, Universal Serial Bus, or other transport. In this manner, the magnetic-conductive mating interface establishes physical and electrical compatibility while the HID profile establishes logical compatibility, together forming a layered standard suitable for interoperable third-party modules and peripherals. The HID profile may be a profile specifically configured for biosignal acquisition platforms, including without limitation a profile providing biosignal channel events, module-identification events, contact-quality events, and stimulation control events; or the HID profile may be a standard HID profile augmented with vendor-defined usage pages associated with biosignal acquisition. Coordination of the disclosed electromechanical interface specification with such an HID profile may be desirable for ecosystem licensing and for designation as a standard-essential interface for head-mounted biosignal acquisition platforms.

The solution disclosed herein may provide a modality-agnostic platform allowing a single device to serve clinical EEG, research fNIRS, stimulation, and consumer use cases via cartridge swap. No conventional platform offers this. The disclosed standardized third-party interface may support a RAMBUS-style licensing posture: revenue from accessory ecosystem independent of device-unit sales. Mechanical and electrical integration in the tensioning element and adjustment dial may each serve dual mechanical-electrical roles, reducing weight and failure points. Tool-free modular reconfiguration via magnetic-conductive mating may be supported, which is clinically important for sterilization, infection control, and workflow speed. Automatic topology detection in the disclosed platform may eliminate manual configuration and human error in clinical settings. Context-aware closed-loop feedback may provide the capability not found in conventional solutions to fuse environmental sensing with biosignal data at the platform layer with modality-adaptive feedback.

For this reason, the disclosed platform may find ready adoption in a number of commercial applications, such as clinical neurology (EEG monitoring, epilepsy diagnostics, sleep studies, intraoperative monitoring, rapid sensor cartridge swap for hygiene compliance), brain-computer interfaces (BCI) (motor-impaired users controlling augmentative and alternative communication (AAC) and assistive technology; steady state visually evoked potential (SSVEP)-based interaction, neurofeedback therapy (attention-deficit/hyperactivity disorder (ADHD), post-traumatic stress disorder (PTSD), anxiety, stroke rehabilitation with closed-loop, context-aware feedback), research neuroscience (multi-modal acquisition (EEG +fNIRS+ultrasound+stimulation) in a portable, field-deployable platform), consumer wellness (meditation, cognitive training, attention monitoring), and defense/military/aerospace (operator state monitoring, covert configurations).

The disclosed platform may further support space and aerospace operations, including microgravity environments, in which the head-mounted device acquires biosignals from astronauts or other crew members performing extravehicular activity, intra-vehicular operator-state monitoring, in-flight cognitive workload assessment, and sleep-and circadian-cycle monitoring during extended missions. In such use cases, the platform may render visual user-interface elements that are world-locked to physical spacecraft systems, body-locked to the user, or head-locked to the user's field of view, such that critical spacecraft functions, monitoring telemetry, and life-support indicators may be accessed without manual input. The platform may further communicate via the standardized accessory interface or via wireless transport with spacecraft instrumentation, robotic effectors, life-support systems, communications equipment, and external systems located outside a pressurized vehicle, thereby enabling hands-free human-spacecraft interaction. Use cases include space medicine, neurological monitoring of crew under microgravity-induced cognitive and physiological changes, and BCI-based control of spacecraft and habitat systems where the use of conventional input devices is impractical.

There are disclosed herein systems and methods for human agency support through an integrated use of context information from the user's environment, the user's historical data such as recorded usage of the system, a body of recorded historical work product, etc., biosensor signals indicating information about the user's physical state and bodily actions, and explicit user input. These are input to a prompt composer capable of taking in these inputs, generating a prompt for a generative AI or generalist agent.

The output of the generative AI or generalist agent may drive an output stage that is able to support and facilitate the human agency of the user based on the output. In this manner, the user may interact with the system with ease and rapidity, the system generating communications to or instructing the actions of supportive and interactive entities surrounding the user, such as other people, robotic aids, smart systems, etc.

With reference to the figures described below, where numerous instances of a particular element are shown, only a representative selection may be called out and numbered. One of ordinary skill in the art will appreciate that more or fewer components such as biosensors, cameras, microphones, speakers, electrodes, etc., may be configured as part of the disclosed solution. Such components may be arranged in a number of ways, and may be present though not indicated or visible in all illustrated views. These illustrations are not intended to be limiting with regard to number and arrangement of sensors or actuators configured in the disclosed solution.

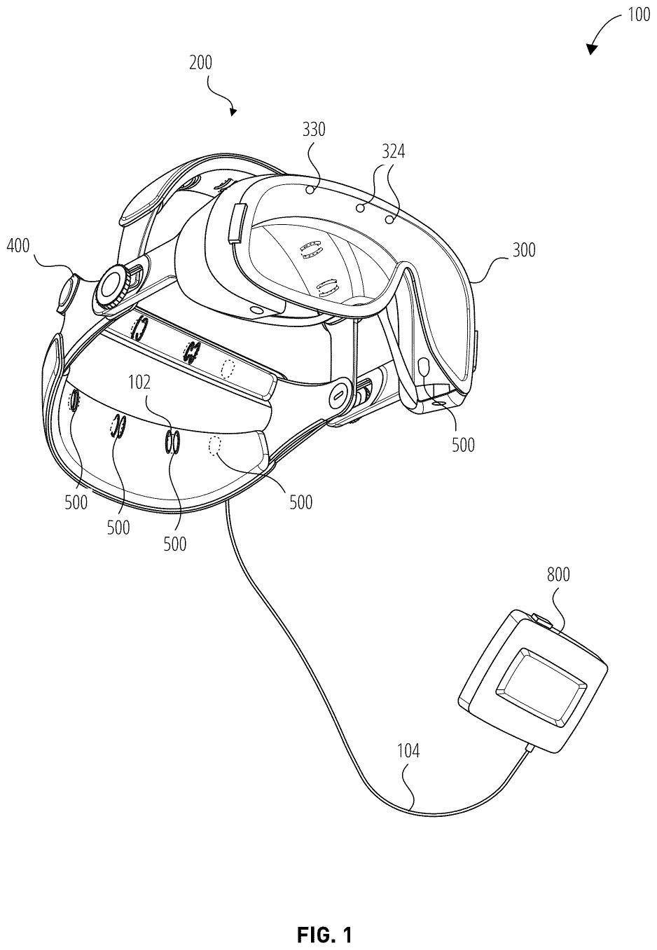

FIG. 1 illustrates an exemplary head-mounted biosignal acquisition platform 100 in accordance with one embodiment. The exemplary head-mounted biosignal acquisition platform 100 may comprise at least one sensor module 102 coupled through a standardized electromechanical mating interface 500 to elements of a modular head-mounted biosignal acquisition system 200, such as a structural chassis assembly 300 and a wearable biosignal acquisition strap assembly 400.

In one embodiment, the modular head-mounted biosignal acquisition system 200 may include brain sensor elements and other biosignal detecting sensors, as well as environmental data collecting sensors such as cameras, microphones, inertial measurement unit (IMU) sensors, etc. In one embodiment, the structural chassis assembly 300 may incorporate at least one camera 324 and at least one microphone 330.

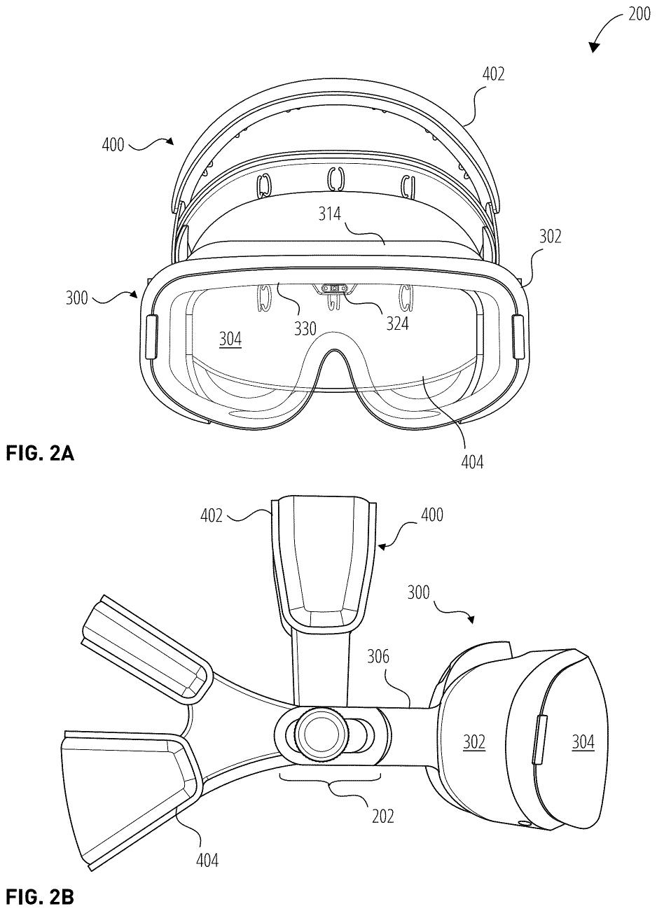

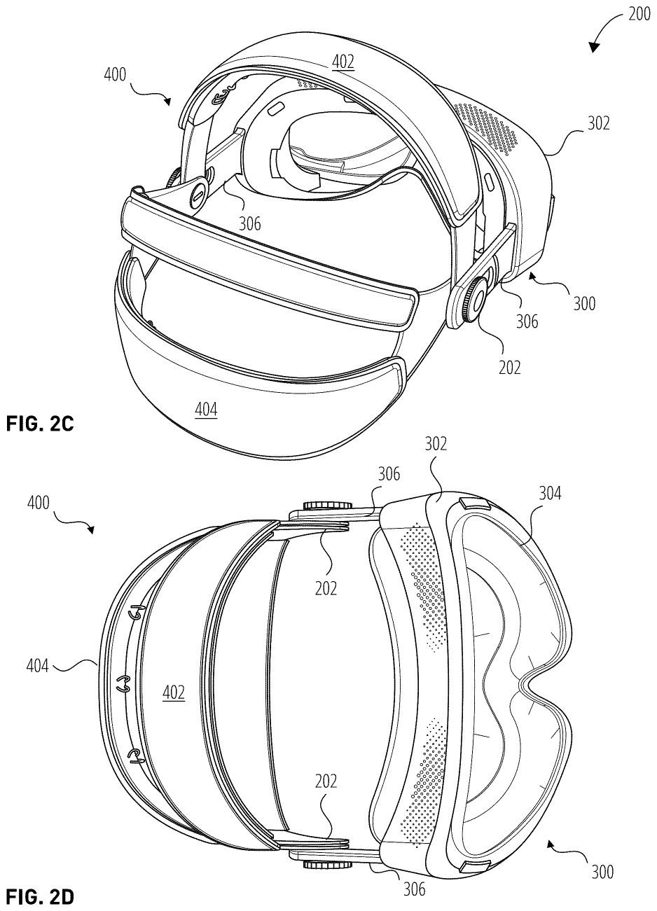

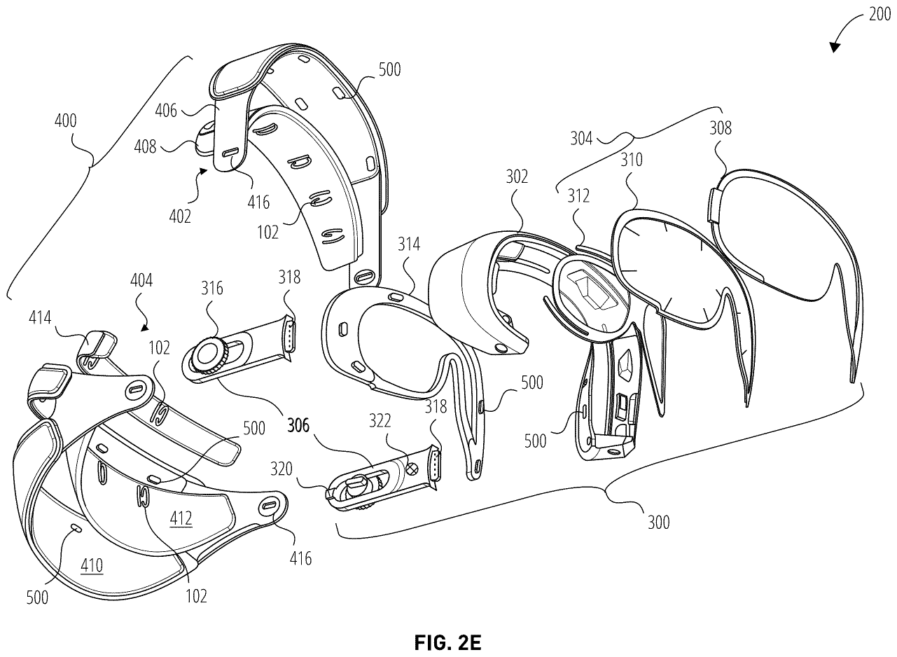

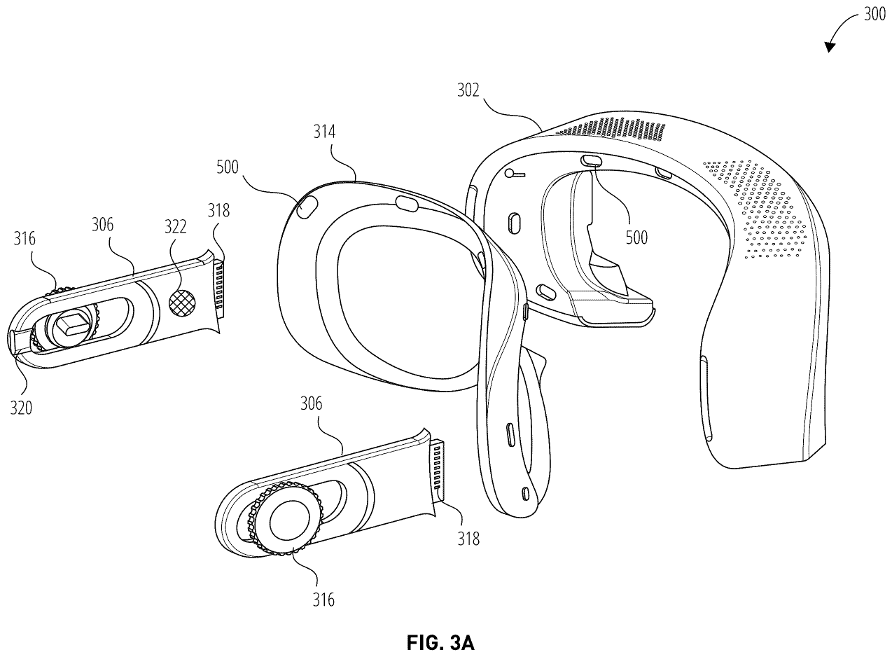

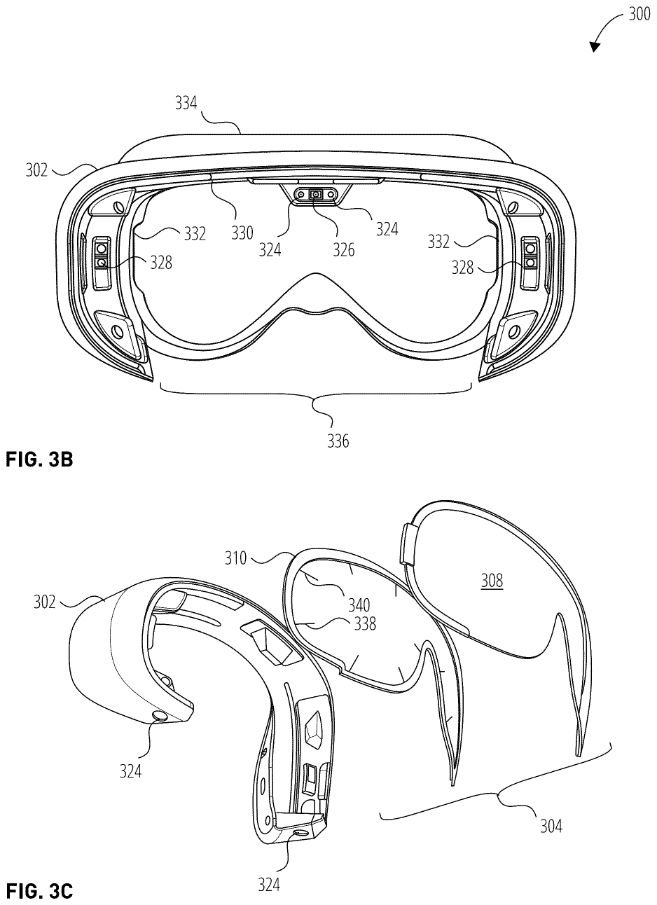

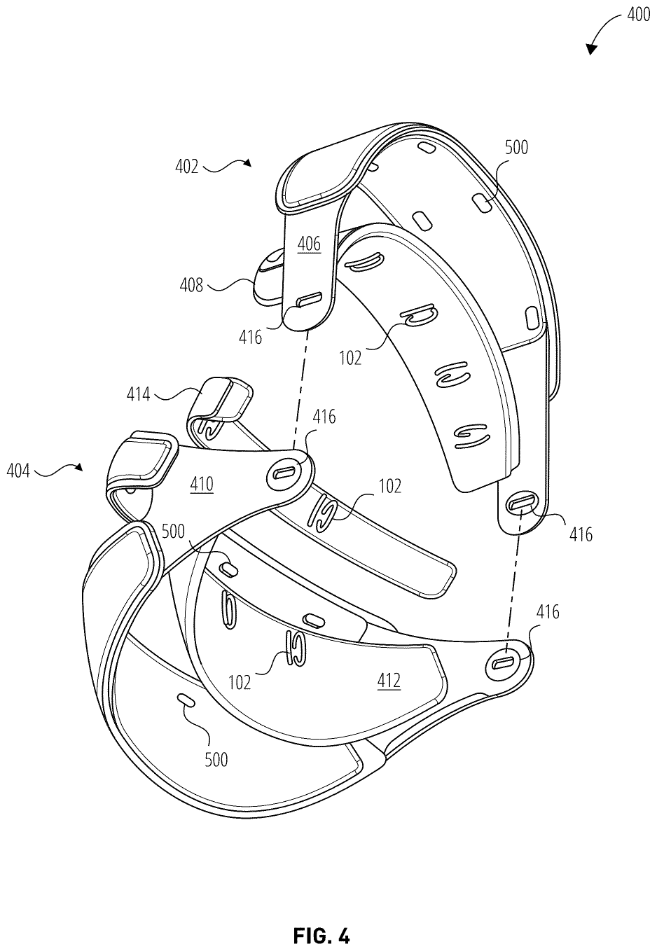

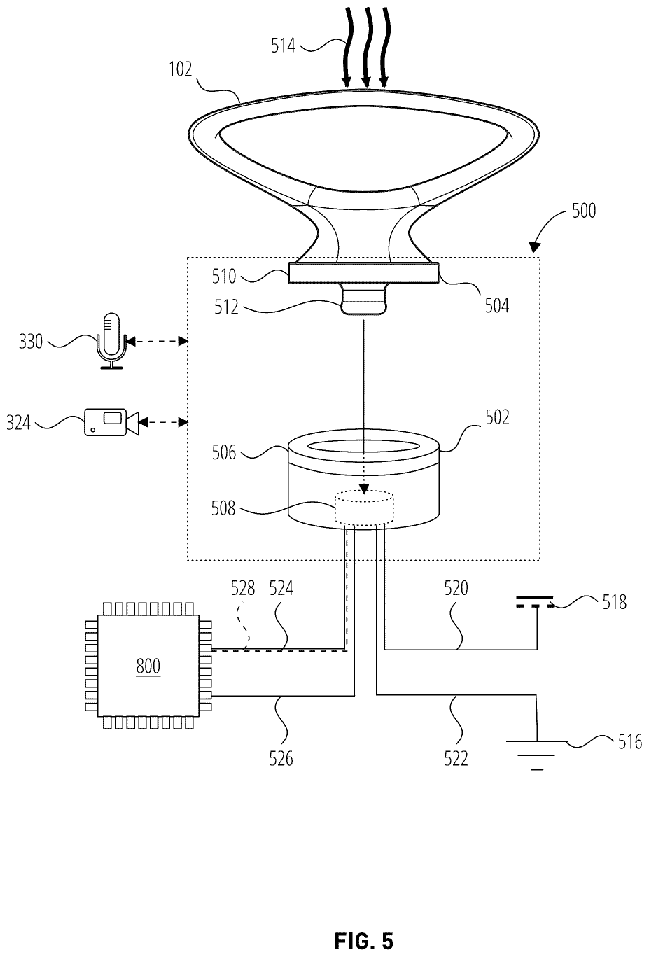

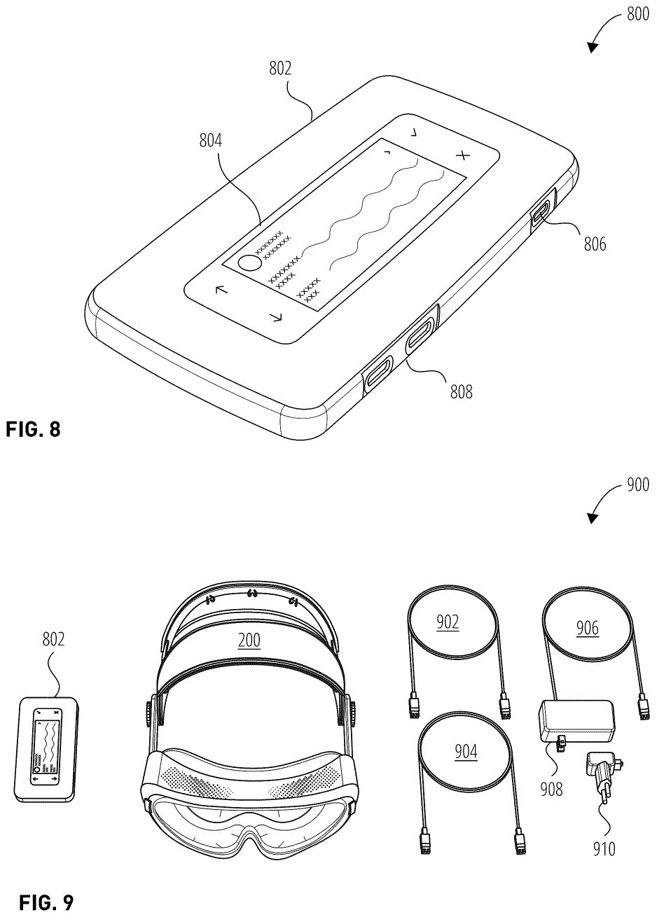

The exemplary head-mounted biosignal acquisition platform 100 may also include a wired coupler 104 connecting the modular head-mounted biosignal acquisition system 200 to a processing system 800. The processing system 800 may be configured to perform signal processing of biosignals acquired by sensor modules 102. These elements are described in greater detail with respect to FIG. 2A-2E, FIG. 3A-3C, FIG. 4, FIG. 5, and FIG. 8 below.

FIG. 2A-2E illustrates a modular head-mounted biosignal acquisition system 200 in accordance with one embodiment. FIG. 2A shows a front elevation view. FIG. 2B shows a right side elevation view. FIG. 2C shows a rear perspective view. FIG. 2D shows a top plan view. FIG. 2E shows an exploded view of components forming the subassemblies comprised in the modular head-mounted biosignal acquisition system 200, which are described in greater detail with respect to FIG. 3A-3C, FIG. 4, and FIG. 5.

The modular head-mounted biosignal acquisition system 200 includes a structural chassis assembly 300 and a wearable biosignal acquisition strap assembly 400 brought together by a tensioning element 202 that interfaces couplable elements such as the rotational dials 316 and tensioning interfaces 416 shown to adjust the relative positions of the structural chassis assembly 300 and wearable biosignal acquisition strap assembly 400, allowing a user to personalize the fit of the modular head-mounted biosignal acquisition system 200 to their own needs.

Various elements of the modular head-mounted biosignal acquisition system 200, and in particular components of the structural chassis assembly 300 and wearable biosignal acquisition strap assembly 400, may incorporate standardized electromechanical mating interfaces 500 allowing the attachment of numerous sensor modules 102. Connections through the standardized electromechanical mating interface 500 or other connection types as are well known to one of ordinary skill may incorporate the connection of at least one camera 324 and at least one microphone 330 through the structural chassis assembly 300 or other portion(s) of the exemplary head-mounted biosignal acquisition platform 100.

FIG. 3A-3C illustrates a structural chassis assembly 300 in accordance with one embodiment. The structural chassis assembly 300 may comprise a structural chassis 302, a front optical lens assembly 304 including an optical see-through lens 308, an eye tracking sensor insert 310 with near-field eye tracking sensors 338, and a prescription lens insert 312. The structural chassis assembly 300 may also include a face sensor insert 314, strap assembly connector arms 306 including rotational dials 316 acting as tensioning elements, electrical contacts 318 to carry signals from components on the structural chassis 302, front optical lens assembly 304, and face sensor insert 314, and one or more peripheral connectors 320 capable of receiving and transmitting signals through the electrical contacts 318. The structural chassis 302 may incorporate cameras 324, projectors 328, microphones 330, and notches 332 allowing a user to wear glasses concurrently with the modular head-mounted biosignal acquisition system 200.

The structural chassis assembly 300 includes a structural chassis 302 that acts as a see-through enclosure surrounding the user's eyes and providing mechanical support/comfort. The structural chassis 302 may couple with a removable face sensor insert 314 that may be disposable. In some embodiments, this insert has no sensing elements and is a non-sensing comfort insert or face pad 334 primarily for comfort, to be coupled to the structural chassis 302 in place of the face sensor insert 314 or other devise including a sensor module.

The strap assembly connector arms 306 may contain electronic elements to support analog or digital sensor signal pathways between the structural chassis 302, front optical lens assembly 304, and face sensor insert 314 and the peripheral connector 320 as well as the modular head-mounted biosignal acquisition system 200 and a computing chip within the modular head-mounted biosignal acquisition system 200 itself or within the processing system 800.

The strap assembly connector arm 306 may include electrical and/or mechanical connectors intended to mate with the structural chassis 302 and couple to the wearable biosignal acquisition strap assembly 400.

In one embodiment, the strap assembly connector arm 306 may be used without the structural chassis 302 as illustrated, but may rather mate with a forehead strap design. In this manner, the modular head-mounted biosignal acquisition system 200 may be configured and used as a biosensing system without need for or attachment to the structural chassis 302 having a front optical lens assembly 304. This head strap design may in one embodiment be a version of the top strap assembly 402 configured with mating connectors configured to accept the strap assembly connector arm 306 rather than the tensioning interface 416 illustrated. In one embodiment, the strap assembly connector arms 306 may include an auditory stimulator 322, such as a speaker. In one embodiment, the auditory stimulator 322 may be incorporated into the structural chassis 302 or a portion of the wearable biosignal acquisition strap assembly 400, as will be well understood by one of ordinary skill in the art.

The face sensor insert 314 and face pad 334 elements may include semi-rigid materials to aid in the mechanical connection to the structural chassis 302. The connection methods may include magnets or other mechanical connection methods.

The structural chassis assembly 300 may include at least one sensor for biometric data of the person, attachable through standardized electromechanical mating interfaces 500 configured within the elements of the structural chassis assembly 300. The system may include at least one biofeedback sensor or actuator (e.g., haptic, sound, electrical current, magnetic field, ultrasound, air pressure, temperature, etc.).

The structural chassis assembly 300 may include a number of sensing and actuating components. The structural chassis 302 may include devices such as cameras configured for various types of light detection and fields of view, such as cameras 324, lidar sensors 326, projectors 328, directional and ambient microphones 330, light emitting diode (LED) indicators, and other input and output source devices. Cameras 324 may be aimed at outward, away from the face, to detect the environment and monitor an approximate representation of the user's field of vision, inward toward the face to monitor expression and gaze, downward to detect body state and motion, additional configurations as needed, and combinations thereof. The system includes at least one projector 328 that may be aimed at least outward towards a medium to be projected upon, or inward directly toward the person's face and eyes, or both.

The structural chassis assembly 300 may include special shapes or notches 332 in the structural chassis 302 to allow room for a user to wear eye glasses inside of the enclosure. The structural chassis assembly 300 may incorporate a variety of removable or adjustable face pads 334 for comfort and stability in addition to or as alternatives to the face sensor insert 314. In one embodiment, the face pad 334 may be a single-use disposable insert.

A lower portion 336 of the structural chassis 302 may be open, allowing a user wearing the system to have relatively unobstructed line of sight downward. For example, seated at a desk while donning the modular head-mounted biosignal acquisition system 200, a user looking forward and sitting in a natural position may be able to look downward and clearly see their hands on the desktop, or while typing or using a mouse. This is impossible with conventional extended reality (XR) and virtual reality (VR) closed-style front assemblies.

Elements of the front optical lens assembly 304 may be configured to mount within the structural chassis 302. The front optical lens assembly 304 may include at least one optical see-through lens 308. In various embodiments, the optical see-through lens 308 may be clear, tinted, or coated. The optical see-through lens 308 may be a replaceable accessory for user cosmetic preference, ultraviolet (UV) protection, polarization, and other optical needs. The optical see-through lens 308 may increase contrast to improve device performance for augmented reality. In one embodiment, the optical see-through lens 308 may be treated to be partially see-through while also acting as a projection surface for the projector 328, which may act as an AR display projector.

The front optical lens assembly 304 may further support an eye tracking sensor insert 310 This may be an additional optical see-through lens incorporating the capability within the lens itself to support near-field eye tracking. Near-field eye tracking sensors 338 alternating with near-field eye tracking emitters 340 may be configured around the perimeter and within or on the surface of the medium of the eye tracking sensor insert 310. The eye tracking sensor insert 310 may also be manufactured in a way to be a medium that receives the light projections from at least one projector 328 inside of the front assembly, rendering light and visual graphics as semi-opaque or translucent holographic images on the inside surface of the lens, such that the wearer of the front assembly may see visual graphics. Thus a single lens insert may contact at least one of eye tracking sensors, and one of augmented reality projected graphics, or both, in the same medium or separate mediums. If separate mediums, or combined, the two technologies of projections and eye tracking may be designed in such a way to be optically visible, and detectable regarding what holograms may be gazed at by the person's eyes.

The front optical lens assembly 304 may include two or more such optical see through lenses. These may be connected via magnets or other mechanical methods, attaching to each other. The combined lenses may be attached to the structural chassis 302 as a unit or independently. In this manner, multiple separate mediums/lenses that have unique features unto themselves may work together at a subsystem. In other embodiments, these separate media may be consolidated into a single medium or lens retaining all functionality in a unified subsystem. In one embodiment, the front optical lens assembly 304 may support an optional prescription lens insert 312 as shown in FIG. 2E.

When fully assembled into the modular head-mounted biosignal acquisition system 200 and worn, the person wearing the headset may see through the elements of the front optical lens assembly 304 in order to visually perceive their surroundings. It may be important that the various coatings and tints on the at least one lens or combination of lenses allows for sufficient visibility of the eyes of the person wearing the system. In some embodiments, it may be important that an external viewer, such a person in proximity to the user, is not able to see the eyes of the person wearing the system. In some embodiments, it may be important that the external viewer be unable to detect any light being emitted within the headset, such as night time, or for special use cases such as military applications. The components of the front optical lens assembly 304 may, individually or as a unit, be configured to support these attributes as needed.

FIG. 4 illustrates a wearable biosignal acquisition strap assembly 400 in accordance with one embodiment. The wearable biosignal acquisition strap assembly 400 may comprise a top strap assembly 402 and a rear strap assembly 404. The top strap assembly 402 may include a top strap frame 406 and a top strap sensing assembly 408. The rear strap assembly 404 may include a rear strap frame 410, a first rear strap sensing assembly 412, and a second rear strap sensing assembly 414. The top strap sensing assembly 408, first rear strap sensing assembly 412, and second rear strap sensing assembly 414 may be configured with sensor modules 102. The sensor modules 102 may be configured to couple electrically to conductors in the top strap frame 406 and rear strap frame 410 through standardized electromechanical mating interfaces 500.

The wearable biosignal acquisition strap assembly 400 may include semi-rigid elements such as the top strap frame 406 and rear strap frame 410 that provide structural support and a mechanical anchor for the top strap sensing assembly 408 and first and second rear strap sensing assemblies (412 and 414), respectively. In one embodiment, sensing elements may be molded within or otherwise integral to these assemblies. In one embodiment, removeable and interchangeable sensor module 102 may be used. In another embodiment, combinations of integral and removable sensor modules 102 may be used. While only some of the sensor module 102 represented in this figure are indicated as such, one of ordinary skill in the art will appreciate that more or fewer sensor modules 102 than are shown, or sensor modules 102 in locations not visible or not indicated, may be included. This illustration is not intended to be limiting with regard to number and arrangement of sensor modules 102.

In one embodiment, the top strap assembly 402 may be removeable. A hinge mechanism may allow users to position the top strap over various brain regions, such as over the sensory or motor cortex area. The top strap assembly 402 may have a bounded rotation ability forward and backwards along the top surface of the head, anchored at the rotational dial 316 or other coupling mechanism of the strap assembly connector arms 306. The range of motion of the top strap assembly 402 may be determined by its configuration and interactions relative to the structural chassis assembly 300 and the rear strap assembly 404.

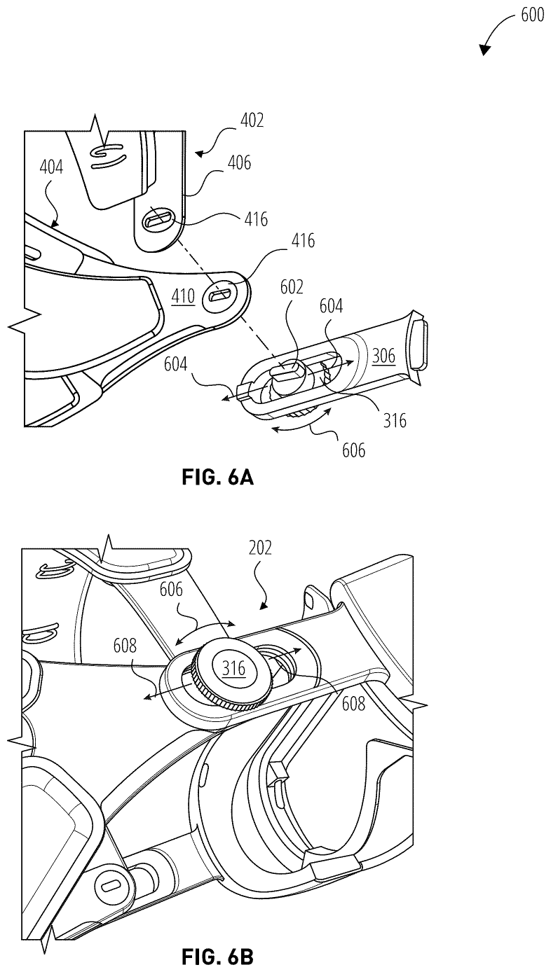

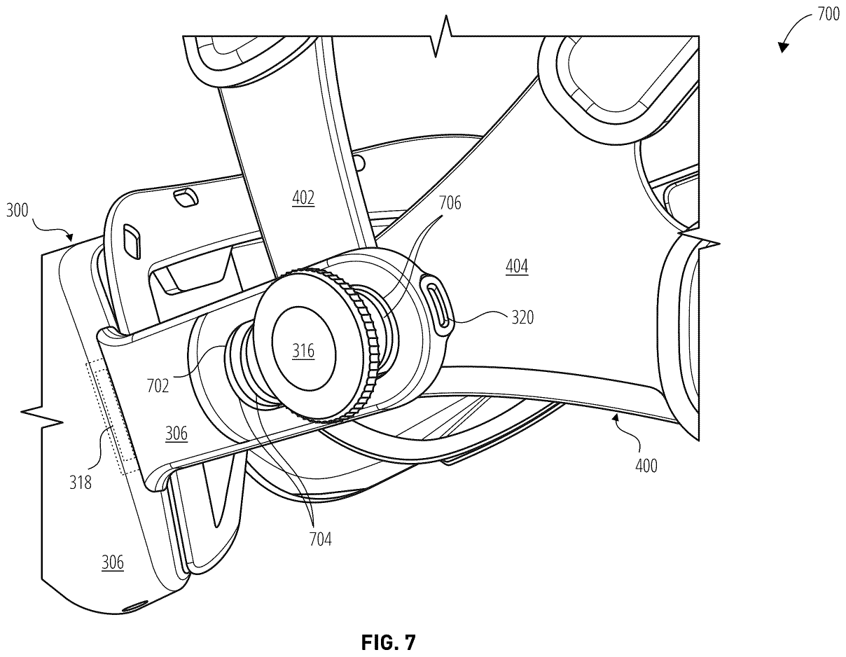

The rear strap assembly 404 may be rotational upward and downward along the back of the head within a bounded range of motion. The rear strap assembly 404 may be anchored at the strap assembly connector arms 306 similarly to the top strap assembly 402. The range of motion of the rear strap assembly 404 may be bounded by the top strap assembly 402 position and the area of the user's head where it meets the cervical spine or rear of the neck. Engagement of the top strap assembly 402 and rear strap assembly 404 with the strap assembly connector arm 306 is described in additional detail with respect to the tensioning element installation and operation 600 illustrated in FIGS. 6A-6B.

The rear strap assembly 404 may be manufactured with sensor features as a single piece, using various manufacturing techniques. In one embodiment, the rear strap assembly 404 may be produced with or without sensors. The rear strap assembly 404 may be produced with at least one material or multiple materials and coatings. The rear strap assembly 404 may be produced with at least one unified durometer, or multiple depending upon areas of the head that may have greater or lesser sensitivity

The wearable biosignal acquisition strap assembly 400 may include textile smart sensing fibers and materials woven into the textile pad for biosensing. In one embodiment, the sensor modules 102 embedded in the wearable biosignal acquisition strap assembly 400 may be molded, cleanable electrodes. In one embodiment, they may be removeable electrodes that may be removed for independent sterilization, and easily replaced when faulty. In one embodiment, the electrodes may be single-use and disposable. In one embodiment, the rear strap assembly 404 may incorporate molded sensor inserts or embedded sensors that are not removeable.

Electrode sensors may be rigid, semi-rigid or flexible. Electrodes may be coated or uncoated. Electrodes may be made of one material or multiple materials. Those materials may be rigid, semi-rigid, soft, flexible, or combination thereof. Electrodes may be made for dry use, or use with additive liquids to hydrate or convert the electrode performance into a wet electrode. Electrodes may have different flexing tolerances or durometer than the integrated pad that it is either manufactured into, or attached or assembled with.

In one embodiment, an adjustment mechanism comprises a plurality of electrical conductors disposed within or integrated into the adjustment mechanism such that the adjustment mechanism forms a continuous electrical signal pathway between the first strap segment and the second strap segment through the adjustment mechanism across a range of rotational positions of the adjustment mechanism. In one embodiment, at least one electrical conductor may be integrated with at least one tensioning element. The tensioning element and the electrical conductor may together form a composite structure that, during operation of the strap assembly, concurrently applies mechanical tension and conducts at least one of a biosignal and a power signal between a sensor module and a processing system.

In one embodiment, the adjustment mechanism may comprise a rotational dial containing at least one of concentric conductive rings and brush contacts configured to maintain electrical continuity across rotational positions. This may allow multi-channel biosignal data to pass through the dial. The at least one tensioning element may in one embodiment comprise at least one of conductive fibers woven or braided around a tension cable, a conductive stainless-steel wire rope with an insulating coating, co-extruded composites, and a flex-circuit ribbon integrated with a tensioning tape.

FIG. 5 illustrates an exemplary standardized electromechanical mating interface 500 in accordance with one embodiment. The standardized electromechanical mating interface 500 may include a first interface member 502 and a second interface member 504 configured to couple securely together both electrically and mechanically. In one embodiment, the first interface member 502 and second interface member 504 may include at least one first electrical contact 508 and at least one second electrical contact 512, respectively. The first interface member 502 and second interface member 504 may further include a first magnet 506 and a second magnet 510, respectively.

The first magnet 506 of the first interface member 502 and the second magnet 510 of the second interface member 504 may be configured to attract each other with increasing force as a sensor module 102 configured with the second interface member 504 is brought into greater proximity to the first interface member 502. In this manner, when the second interface member 504 is mated to the first interface member 502, the first magnet 506 and second magnet 510 may provide mechanical retention force to hold the sensor module 102 securely mechanically coupled to whatever element is equipped with the first interface member 502 of the standardized electromechanical mating interface 500.

The first electrical contact 508 of the first interface member 502 may be configured to accept and conduct electrical signals from the second electrical contact 512 when the two are seated against each other or one within the other, as will be readily understood one of ordinary skill in the art. Their coupled electrical contact may be secured and supported by the mechanical coupling of the magnets described above. In this manner, the sensor module 102 may detect biosignals 514 or other detectable signals, and may transmit these electrically through the second electrical contact 512 to the first electrical contact 508. The first electrical contact 508 may electrically couple to ground 516 and power 518 through a power conductor 520 and a ground conductor 522, respectively. In this manner, the standardized electromechanical mating interface 500 may supply power to a mounted sensor module 102. The first electrical contact 508 may further be eclectically coupled to a biosignal channel conductor 524 and a digital identification conductor 526. These conductors may be in wired communication with a processing system 800. In one embodiment, the biosignal channel conductor 524 and digital identification conductor 526 may be in wired communication with a network device supporting wireless communication with a processing system 800.

In one embodiment, at least one camera 324 and at least one microphone 330 may be configured to connect to the processing system 800 through conventional connectors and conductors that will be well understood by one of ordinary skill in the art. In one embodiment, these devices may be configured to connect through the standardized electromechanical mating interface 500, as illustrated. The biosignal channel conductor 524 may further act as a general signal channel conductor 528 in such embodiments. Other devices than specifically biosignal sensor modules may thus be incorporated into an exemplary head-mounted biosignal acquisition platform 100 using the standardized electromechanical mating interface 500 disclosed herein.

In one embodiment, the standardized electromechanical mating interface 500 may conform with an accessory interface specification for a head-mounted device. The accessory interface specification may include a physical connector geometry, a mechanical retention specification, an electrical pin assignment defining at least one power conductor, at least one ground conductor, at least one biosignal channel conductor or general or other signaling channel conductor, and at least one digital identification conductor, and. The accessory interface specification may also include a module-identification signaling protocol. The accessory interface specification may be configured such that any module conforming to the physical connector geometry, mechanical retention specification, electrical pin assignment, and module-identification signaling protocol is physically and electrically engageable with a head-mounted device implementing the accessory interface specification without modification of the head-mounted device. The module-identification signaling protocol may be configured to identify a module type and a capability profile of the conforming module to a processing system of the head-mounted device.

In one embodiment, the head-mounted biosignal acquisition platform may be configured to alternate between acquisition and stimulation modes by the same standardized electromechanical mating interface 500. In such an embodiment, the signal channel conductor 528 or biosignal channel conductor 524 may carry signals to a device coupled to the standardized electromechanical mating interface 500 as is shown for the sensor module 102 such that the device provides stimulation in addition to passive signal acquisition.

FIG. 6A-6B illustrates tensioning element installation and operation 600 in accordance with one embodiment. FIG. 6A illustrates the manner in which one side of a top strap assembly 402 and rear strap assembly 404 may be configured to connect with a rotational dial 316 or other mechanism of the strap assembly connector arm 306 in order to bring the wearable biosignal acquisition strap assembly 400 and structural chassis assembly 300 into mechanical and electrical contact and communication.

A tensioning key 602 may be configured on an end of a rotational dial 316 opposite the end manipulable by the user. This tensioning key 602 may fit within or otherwise engage with the tensioning interface 416 of the rear strap frame 410. The tensioning key 602 may pass through this tensioning interface 416 in order to further engage with the tensioning interface 416 of the top strap frame 406.

The rotational dial 316 may be configured within a slot or track in the strap assembly connector arm 306. Through a rolling or rotational motion 606 of the rotational dial 316, the rotational dial 316 may be configured to traverse 604 a linear path within this slot or track. Additional description of this is provided with respect to the strap assembly connector arm details 700 of FIG. 7.

FIG. 6B illustrates the operational interaction of the strap assembly connector arm 306, top strap assembly 402, and rear strap assembly 404 when fully engaged to form the tensioning element 202. In this configuration, the rolling or rotational motion 606 of the rotational dial 316 and its resulting traverse 604 along the track or slot within the strap assembly connector arm 306 allow tension and fit adjustment 608 of the conjoined components by the tensioning element 202.

In one embodiment, the rotational dial 316 of the tensioning element 202 may support at least one degree of rotational adjustment. Mechanical parts of the tensioning element 202 may be made of rigid, semi-rigid, and/or flexible materials, and combination, thereof, to increase usability and comfort of the system. The tensioning element 202 supports an integrated strap adjustment system that allows for positioning of both the top strap and the rear strap. This configuration may provide an integration point for 3^{rd} party headsets in one embodiment.

The tensioning element 202 may in various embodiments be implemented as or with internal belts, cables, or moving parts that allow one or more connected straps to be adjusted as a whole together, or independently. The tensioning element 202 may include additional features supporting incremental adjustments ranging from smooth, fluid rotation or linear motion to ratcheted positions when rotated or otherwise displaced. In one embodiment the rotational dial 316 may be in a fixed position relative to its distance toward or away from the user's head. In one embodiment, the rotational dial 316 may be capable of being pressed inward or pulled outward to initiate secondary adjustment features.

In one embodiment, the tensioning element 202 may have a locking mechanism. Lock and unlock features may be engaged or disengaged to engage or disengage rotation of the dial, linear motion of belts or cables, etc. These features may be pressed, grasped, squeezed, toggled, or otherwise manipulated by the user.

FIG. 7 illustrates a strap assembly connector arm details 700 in accordance with one embodiment. The rotational dial 316 of the afore-described tensioning element 202 is shown situated within a track or slot 702 configured lengthwise along a portion of the strap assembly connector arm 306. The rotational dial 316 is shown engaged with the top strap assembly 402 and rear strap assembly 404, thus securing the structural chassis assembly 300 to the wearable biosignal acquisition strap assembly 400.

The track or slot 702 may be configured with depth-offset conductive channels 704 along its inner perimeter. These depth-offset conductive channel 704 may be configured to accept concentric ring contacts 706 configured around the portion of the rotational dial 316 residing within the track or slot 702. In this manner, or in analogous configurations that will be readily understood by one of ordinary skill in the art, the motive mechanism of the tensioning element 202 may be provisioned with metal contacts that support electrical connection among the strap assembly connector arm 306, top strap assembly 402, and rear strap assembly 404, allowing passage of signals generated by any sensors configured within these elements to a centralized physical location for processing and/or busing to additional devices.

In this manner, the tensioning element 202 may also be designed to act as a connection hub for embedded electronics, sensors and actuators in the structural chassis assembly 300 and wearable biosignal acquisition strap assembly 400. Signals from the structural chassis 302 may pass through the electrical contact 318 to this hub, which may further be in communication with the peripheral connector 320 and devices connected through it. This configuration may support data transfer from strap to another to a computer chip, either inside the headset or detached from the headset, connected wirelessly or via a cable.

FIG. 8 illustrates a processing system 800 in accordance with one embodiment. The exemplary head-mounted biosignal acquisition platform 100 may include the processing system 800 as an external hip-pack accessory 802 that contains computer, battery, wireless, analog signal processing, and a touch display 804. The hip-pack accessory 802 may optionally include an isolated trigger input 806 for precise synchronization. The hip-pack accessory 802 may optionally include a general purpose analog and/or digital interface 808 for additional peripherals. The touch display 804 may be used for visualization of acquisition and/or for clinical application deployments for patient or clinician use. The processing system 800 may, in addition or alternatively, be distributed across mulitple processing components, including those in the modular head-mounted biosignal acquisition system 200, those in the hip-pack accessory 802, those configured on additional computational hardware local to a user, and those available via a cloud computing system.

FIG. 9 illustrates exemplary modular biosignal acquisition platform components 900 in accordance with one embodiment. The exemplary modular biosignal acquisition platform components 900 may comprise the modular head-mounted biosignal acquisition system 200 and hip-pack accessory 802 (or other processing system 800) previously described, as well as a USB-C cable 902, a Lightning cable 904, a USB-A cable 906, a US power adapter 908, and an EU power adapter 910. One of ordinary skill in the art will appreciate that this list is not exhaustive and is intended as a non-limiting example. Numerous sensing, active, connective, and power components may be included in the solution disclosed herein.

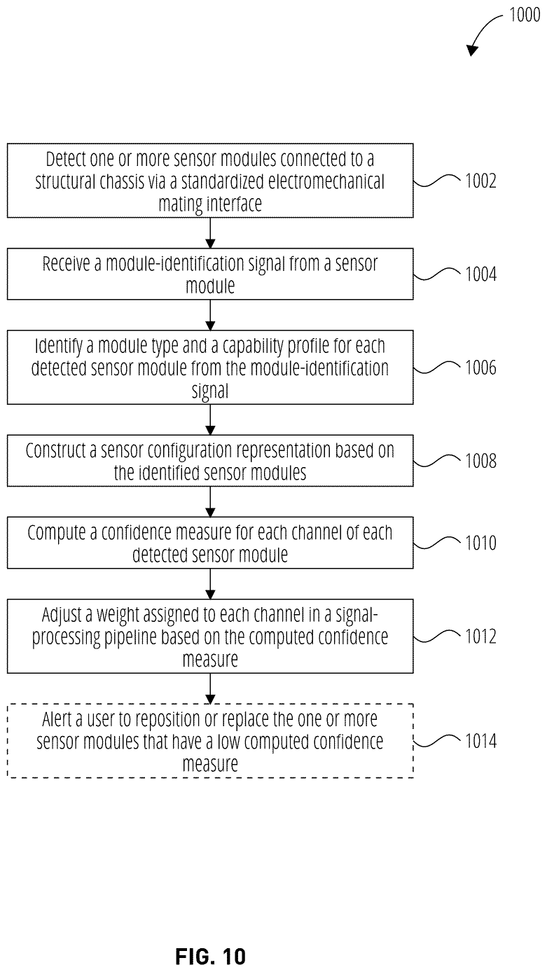

FIG. 10 illustrates an example routine 1000 to be performed by the exemplary head-mounted biosignal acquisition platform 100. Although the example routine 1000 depicts a particular sequence of operations, the sequence may be altered without departing from the scope of the present disclosure. For example, some of the operations depicted may be performed in parallel or in a different sequence that does not materially affect the function of the routine 1000. In other examples, different components of an example device or system that implements the routine 1000 may perform functions at substantially the same time or in a specific sequence.

According to some examples, the method includes detecting one or more sensor modules connected to a structural chassis via a standardized electromechanical mating interface at block 1002. For example, the processing system 800 illustrated in FIG. 8 may detect one or more sensor modules connected to a structural chassis via a standardized electromechanical mating interface. The processing system is coupled to the structural chassis of a head-mounted device.

According to some examples, the method includes receiving a module-identification signal from a sensor module at block 1004. For example, the standardized electromechanical mating interface 500 illustrated in FIG. 5 may receive a module-identification signal from a sensor module.

According to some examples, the method includes identifying a module type and a capability profile for each detected sensor module from the module-identification signal at block 1006. For example, the processing system 800 illustrated in FIG. 8 may identify a module type and a capability profile for each detected sensor module from the module-identification signal. In one embodiment, identifying the module type and the capability profile further comprises reading module-identification metadata configured to report calibration metadata to a host platform.

According to some examples, the method includes constructing a sensor configuration representation based on the identified sensor modules at block 1008. For example, the processing system 800 illustrated in FIG. 8 may construct a sensor configuration representation based on the identified sensor modules. In one embodiment, detecting the one or more sensor modules and constructing the sensor configuration representation occur automatically upon power-on of the platform or upon connection or removal of the one or more sensor modules.

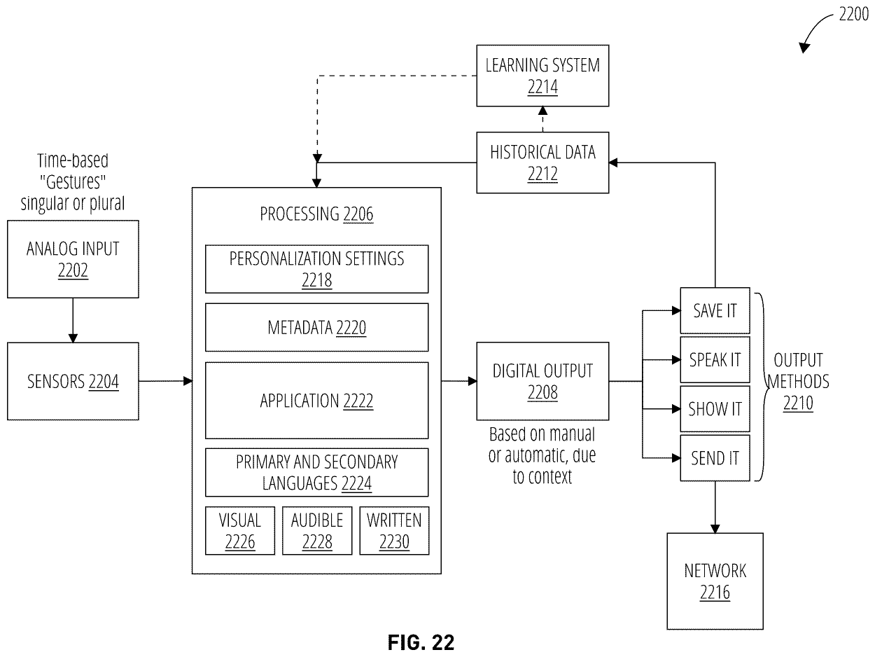

According to some examples, the method includes computing a confidence measure for each channel of each detected sensor module at block 1010. For example, the processing 2206 illustrated in FIG. 22 may compute a confidence measure for each channel of each detected sensor module. The confidence measure may be computed based on at least one of electrical impedance, signal variance, power-line interference, and contact-quality metric. In one embodiment, computing the confidence measure comprises analyzing contact-quality proxies associated with each channel of each detected sensor module.

According to some examples, the method includes adjusting a weight assigned to each channel in a signal-processing pipeline based on the computed confidence measure at block 1012. For example, the processing system 800 illustrated in FIG. 8 may adjust a weight assigned to each channel in a signal-processing pipeline based on the computed confidence measure. In one embodiment, adjusting the weight comprises reducing weighting in a signal-processing model for channels with a low computed confidence measure.

According to some examples, the method includes alerting a user to reposition or replace the one or more sensor modules that have a low computed confidence measure at block 1014. For example, the processing system 800 illustrated in FIG. 8 may alert a user to reposition or replace the one or more sensor modules that have a low computed confidence measure. Alerts may be provided via at least one of a visual augmented reality overlay, an audio tone, and a haptic cue.

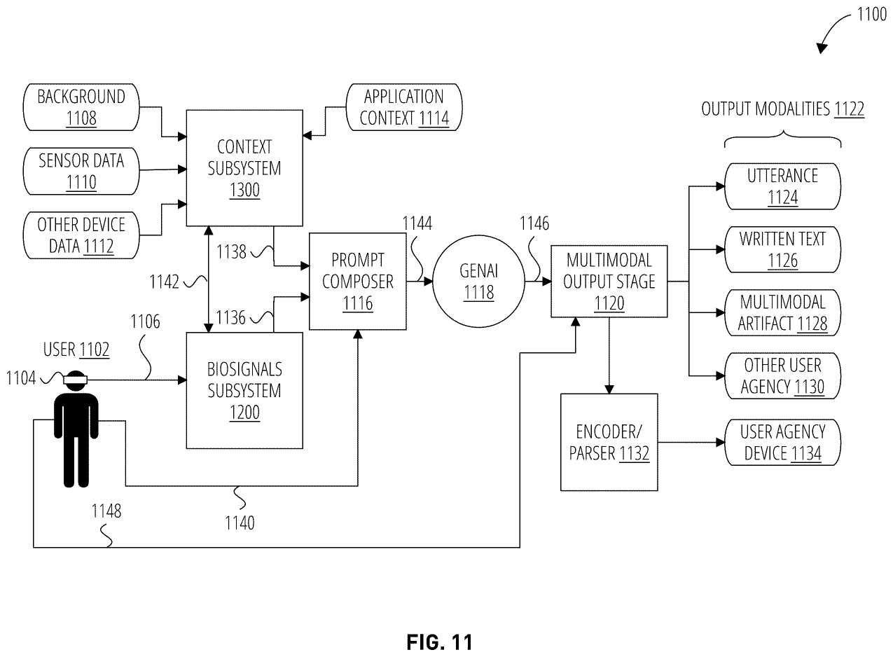

FIG. 11 illustrates a user agency and capability augmentation system 1100 in accordance with one embodiment. The user agency and capability augmentation system 1100 comprises a user 1102, a wearable computing and biosignal sensing device 1104, biosignals 1106, background material 1108, sensor data 1110, other device data 1112, application context 1114 a prompt composer 1116, a GenAI 1118, a multimodal output stage 1120, an encoder/parser 1132, output modalities 1122 such as an utterance 1124, a written text 1126, a multimodal artifact 1128, an other user agency 1130, and a non-language user agency device 1134, a biosignals subsystem 1200, and a context subsystem 1300.

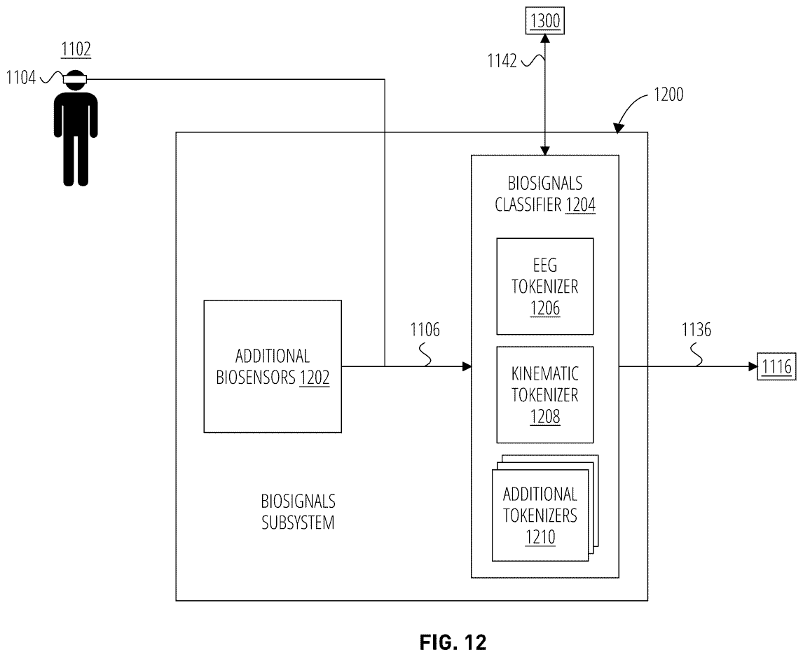

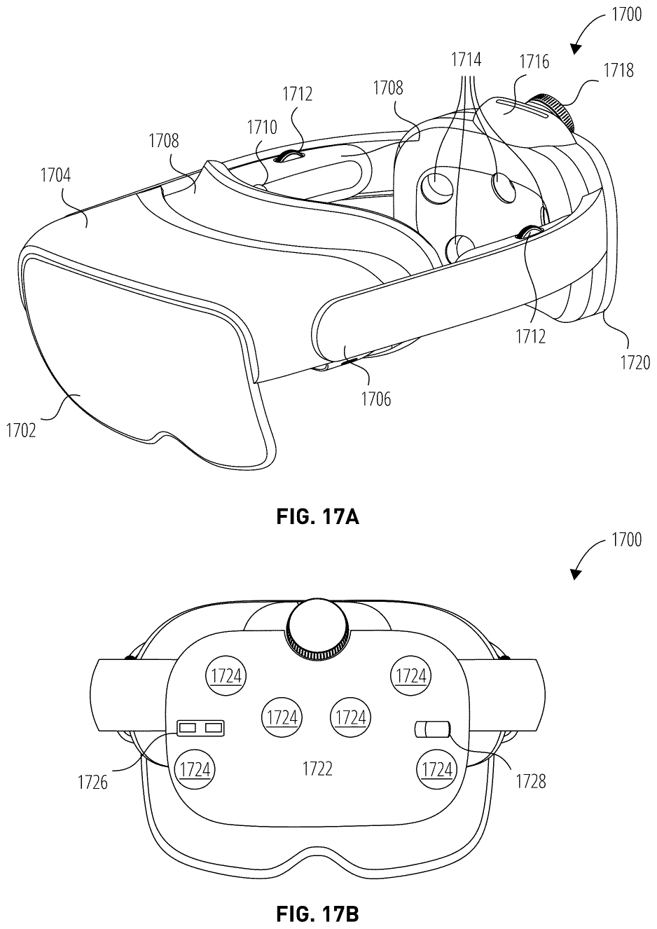

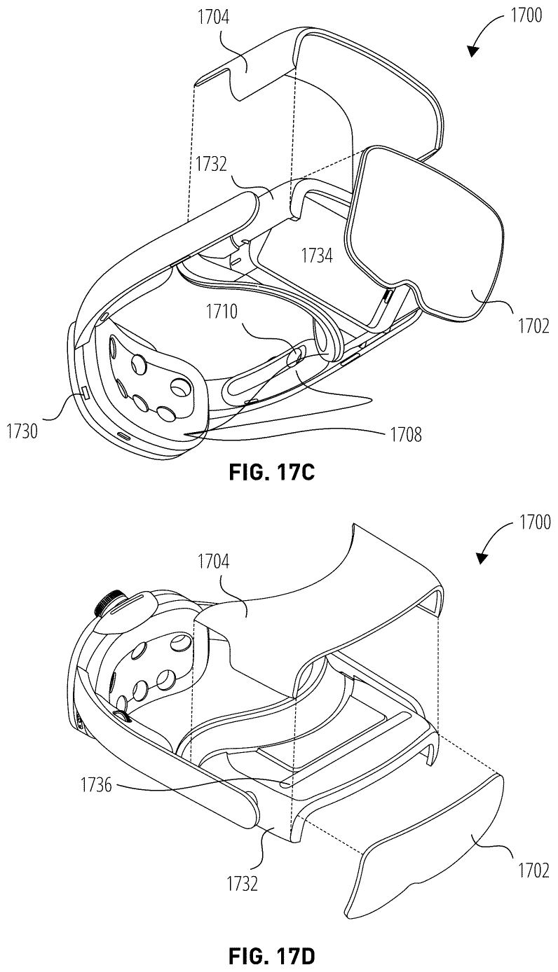

The user 1102 in one embodiment may be equipped with and interact with a wearable computing and biosignal sensing device 1104. The wearable computing and biosignal sensing device 1104 may be a device such as the brain computer interface or BCI headset system 1700 described in greater detail with respect to FIG. 17A through FIG. 17D. This embodiment may provide the user 1102 with capability augmentation or agency support by utilizing biosignals 1106, such as neurologically sensed signals and physically sensed signals, detected from the wearable computing and biosignal sensing device 1104 and sent to a biosignals subsystem 1200, in addition to data from biosignal sensors that may be part of the biosignals subsystem 1200. The biosignals subsystem 1200 may produce as its output a tokenized biosignals prompt 1136. The action of the biosignals subsystem 1200 is described in detail with respect to FIG. 12.

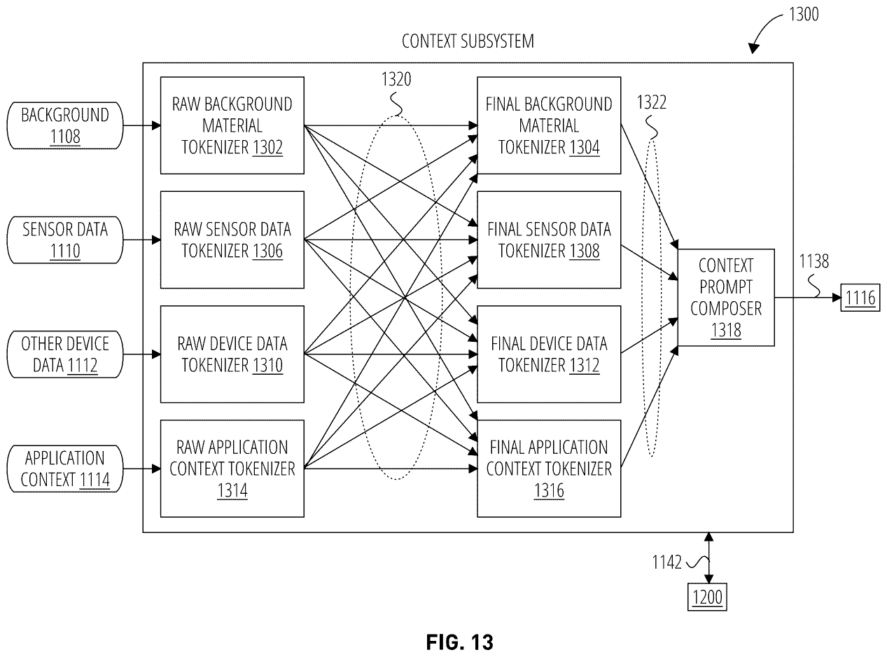

This embodiment may provide the user 1102 with capability augmentation or agency support by utilizing inference of the user's environment, physical state, history, and current desired capabilities as a user context, to be gathered at a context subsystem 1300, described in greater detail with respect to FIG. 13. This data may be provided as background material 1108 on the user stored in a database or other storage structure, sensor data 1110 and other device data 1112 from a range of devices and on-device and off-device sensors, and application context 1114 provided by applications, interfaces, or parameters configured to provide the capability augmentation sought by the user 1102. The context subsystem 1300 may produce as its output a tokenized context prompt 1138.

In one embodiment, the biosignals subsystem 1200 and the context subsystem 1300 may be coupled or configured to allow shared data 1142 to flow between them. For instance, some sensor data 1110 or other device data 1112 may contain biosignal information that may be useful to the biosignals subsystem 1200. Or the biosignals subsystem 1200 may capture sensor data 1110 indicative of the user 1102 context. These systems may communicate such data, in raw, structured, or tokenized forms, between themselves using wired or wireless communication. In one embodiment, these systems may operate as part of a device that is also configured and utilized to run other services.

This embodiment may finally provide the user 1102 capability augmentation or agency support by utilizing direct user 1102 input in the form of a user input prompt 1140, such as mouse, keyboard, or biosignal-based selections, typed or spoken language, or other form of direct interaction the user 1102 may have with a computational device that is part of or supports the user agency and capability augmentation system 1100 disclosed. In one embodiment, the user 1102 may provide an additional token sequence in one or more sensory modes, which may include a sequence of typed or spoken words, an image or sequence of images, and a sound or sequence of sounds. The biometric and optional multimodal prompt input from the user may be tokenized using equivalent techniques as for the context data.

The biosignals prompt 1136, context prompt 1138, and user input prompt 1140 may be sent to a prompt composer 1116. The prompt composer 1116 may consume the data including the biosignals prompt 1136, context prompt 1138, and user input prompt 1140 tokens, and may construct a single token, a set of tokens, or a series of conditional or unconditional commands suitable to use as a prompt 1144 for a GenAI 1118 such as a Large Language Model (LLM), a Generative Pre-trained Transformer (GPT) like GPT-4, or a generalist agent such as Gato. For example, a series such as “conditional on command A success, send command B, else send command C” may be built and sent all at once given a specific data precondition, rather than being built and sent separately.

The prompt composer 1116 may also generate tokens that identify a requested or desired output modality (text vs. audio/visual vs. commands to a computer or robotic device, etc.) from among available output modalities 1122 such as those illustrated. In one embodiment, the prompt composer 1116 may further generate an embedding which may be provided separately to the GenAI 1118 for use in an intermediate layer of the GenAI 1118. In another embodiment, the prompt composer 1116 may generate multiple tokenized sequences at once that constitute a series of conditional commands. In one exemplary use case, the user 1102 submits a general navigational command to an autonomous robot or vehicle, such as “go to the top of the hill.” The prompt composer 1116 may then interact with satellite and radar endpoints to construct specific motor commands, such as “Move forward 20 feet and turn left,” that navigate the robot or vehicle to the desired destination.

In one exemplary use case, the context subsystem 1300 may generate a context prompt 1138 token sequence corresponding to the plaintext, “The user has travelled to Los Angeles to visit a doctor specializing in rare diseases. The user is sitting in the doctor's office and preparing to discuss their disease. The user is looking at the doctor who has just asked the user for an update on their condition.” Such a context prompt 1138 may be generated by utilizing sensors on a computing device worn or held by the user 1102, such as a smart phone or the wearable computing and biosignal sensing device 1104. Such sensors may include global positioning system (GPS) components, as well as microphones configured to feed audio to a speech to text (STT) device or module in order to identify the doctor and the questions. The biosignals subsystem 1200 may generate a biosignals prompt 1136 including a token sequence corresponding to the user selecting “speak” with a computing device to select this directive using an electroencephalography-based brain computer interface. The user input prompt 1140 may include a token sequence corresponding to the plaintext, “The user has selected ‘summarize my recent disease experience’.” In this case, the prompt composer 1116 may simply append these three token sequences into a single prompt 1144 and may then pass it to the GenAI 1118. In an alternate embodiment, the prompt composer 1116 may replace the biosignals prompt 1136 with a token sequence corresponding to the plaintext “Generate output in a format suitable for speech synthesis.”

In some embodiments, the prompt composer 1116 may utilize a formal prompt composition language such as Microsoft Guidance. In such a case, the composition language may utilize one or more formal structures that facilitate deterministic prompt composition as a function of mixed modality inputs. For example, the prompt composer 1116 may contain subroutines that process raw signal data and utilize this data to modify context prompt 1138 and/or biosignals prompt 1136 inputs in order to ensure specific types of GenAI 1118 outputs.

A more intricate exemplary prompt from the prompt composer 1116 to the GenAI 1118, incorporating information detected from user 1102 context and biosignals 1106, may be as follows:

I am Sarah, a 60-year-old retired school teacher with advanced ALS. I enjoy the peaceful sounds of birds chirping. I just finished reading a mystery novel recommended by my friend Donna. As a literature enthusiast, I have a long history of discussing books with my friends and family I need help communicating and you are my assistant.

This is the current context:
Conversation history: Recent discussions about books with family and friends, including favorite authors, genres, and specific titles
Personal preferences: Fondness for mystery novels, historical fiction, and biographies; appreciation for strong character development and engaging plots
Language corpus and demographics: 60-year-old, retired school teacher, well-versed in literary terms and expressions
Mood: Content, relaxed, and eager to share her thoughts on the novel
Conversation Partner: Donna, my friend with shared interest in literature
Environmental audio: Sounds of birds chirping, rustling leaves, and distant neighborly conversations in the garden
Front-facing camera: Images of blooming flowers, lush greenery, and the mystery novel's cover
Location, motion, and positioning: At home, sitting in a comfortable garden chair
Reading preferences: Mystery novels, historical fiction, biographies, and classic literature
Educational background: Years of teaching experience in literature, familiarity with various literary periods and styles.

If I send an emoji, use it to topically or thematically improve prediction and alter tone. Use all contextual information and prior conversation history to modulate your responses. After each input, review the prior inputs and modify your subsequent predictions based on the context of the thread. Taking into account the current context, with spartan language, return a JSON string called ‘suggestions’ with three different and unique phrases without quotes. They should be complete sentences longer than two words. Do not include explanations. The phrases you respond with will be spoken by my speech generating device.

The GenAI 1118 may take in the prompt 1144 from the prompt composer 1116 and use this to generate a multimodal output 1146. The GenAI 1118 may consist of a pre-trained machine learning model, such as GPT. The GenAI 1118 may generate a multimodal output 1146 in the form of a token sequence that may be converted back into plaintext, or which may be consumed by a user agency process directly as a token sequence. In an alternate embodiment, the output of the GenAI 1118 further constitutes embeddings that may be decoded into multimodal or time-series signals capable of utilization by agency endpoints. Once determined, the output is digitally communicated to an agency endpoint capable of supporting the various output modalities 1122.

In some embodiments, the GenAI 1118 may generate two or more possible multimodal outputs 1146 and the user 1102 may be explicitly prompted at the multimodal output stage 1120 to select between the choices. In the case of language generation, the user 1102 may at the multimodal output stage 1120 select between alternative utterances 1124. In the case of robot control, the choices may consist of alternative paths that a robot could take in order to achieve a user-specified goal. In these embodiments, there may be an output mode selection signal 1148 provided by the user 1102 explicitly or indicated through biosignals 1106, to the multimodal output stage 1120. The output mode selection signal 1148 may instruct a choice between the multimodal outputs 1146 available from the GenAI 1118 at the multimodal output stage 1120. In one embodiment, the user 1102 may further direct one or more of the alternatives to alternate endpoints supporting the various output modalities 1122. For example, the user 1102 may select one utterance 1124 for audible presentation and a different one for transformation and/or translation to written text 1126.

In an alternate configuration, the user agency and capability augmentation system 1100 may contain multiple GenAIs 1118, each of which is pre-trained on specific application, context, or agency domains. In this configuration, the context subsystem 1300 may be responsible for selecting the appropriate GenAI 1118 or GenAIs 1118 for the current estimated user context. In some embodiments, mixture-of-experts models such as a generalist language model may be used for this.

In some embodiments, models may be fine-tuned by the user 1102. For example, the user 1102 may provide a GenAI 1118 LLM classifier model with exemplars of classes, either by speaking or writing them, and through few-shot learning, the model may improve accuracy. The multimodal outputs 1146 may be made available to the user 1102 through the agency endpoints supporting the various output modalities 1122, and the user 1102 may respond in a manner detectable through the user's biosignals 1106, or directly through an additional user input prompt 1140, and in this manner may also provide data through which the GenAI 1118 may be refined.

The multimodal outputs 1146 may be used to extend and support user 1102 agency and augment user 1102 capability into real and virtual endpoints. In one embodiment, the selected user agency process may be a speech synthesis system capable of synthesizing a token sequence or text string as a spoken language utterance 1124 in the form of a digital audio signal. In another embodiment, the system's output may be constrained to a subset of domain-relevant utterances 1124 for applications such as employment, industry, or medical care. This output constraint may be implemented using a domain specific token post-processing system or it may be implemented with an alternate GenAI that has been pre-trained on the target domain. In another embodiment, the endpoint may be a written text 1126 composition interface associated with a communication application such as email, social media, chat, etc., or presented on the user's or their companions' mobile or wearable computing device. In a further embodiment, the output may be a multimodal artifact 1128 such as a video with text, an audio file, etc. In another embodiment, the output may augment some other user agency 1130, such as by providing haptic stimulation, or through dynamic alteration of a user's interface, access method, or complexity of interaction, to maximize utility in context.

In some embodiments, the multimodal outputs 1146 may be additionally encoded using an encoder/parser 1132 framework such as an autoencoder. In this system, the output of the encoder/parser 1132 framework may be a sequence of control commands to control a non-language user agency device 1134 or robotic system such as a powered wheelchair, prosthetic, powered exoskeleton, or other smart, robotic, or AI-powered device. In one embodiment, the prompt 1144 from the prompt composer 1116 may include either biosignals prompt 1136 or user input prompt 1140 tokens which represent the user's desired configuration, and the multimodal output includes detailed steps that a robotic controller may digest, once encoded by the encoder/parser 1132. In this embodiment, the user 1102 may express a desire to move from location A to location B, and the combination of the GenAI 1118 and the robot controller may generate an optimal path as well as detailed control commands for individual actuators. In another embodiment, biosignals 1106 may be used to infer a user's comfort with the condition of their surroundings, their context indicating that they are at home, and a prompt may be developed such that the GenAI 1118 provides multimodal outputs 1146 instructing a smart home system to adjust a thermostat, turn off music, raise light levels, or perform other tasks to improve user comfort. In a further embodiment, the GenAI 1118 may generate a novel control program which is encoded by parsing or compiling it for the target robot control platform at the encoder/parser 1132. The multimodal output 1146 may through these methods be available as information or feedback to the user 1102, through presentation via the wearable computing and biosignal sensing device 1104 or other devices in the user's immediate surroundings. The multimodal output 1146 may be stored and become part of the user's background material 1108. The user 1102 may respond to the multimodal output 1146 in a manner detectable through biosignals 1106, and thus a channel may be provided to train the GenAI 1118 based on user 1102 response to multimodal output 1146.

In general, the user agency and capability augmentation system 1100 may be viewed as a kind of application framework that uses the biosignals prompt 1136, context prompt 1138, and user input prompt 1140 sequences to facilitate interaction with an application, much as a user 1102 would use their finger to interact with a mobile phone application running on a mobile phone operating system. Unlike a touchscreen or mouse/keyboard interface, this system incorporates real time user inputs along with an articulated description of their physical context and historical context to facilitate extremely efficient interactions to support user agency. FIG. 11 shows the pathways signals take from input, by sensing devices, stored data, or the user 1102, to output in the form of text-to-speech utterances 1124, written text 1126, multimodal artifacts 1128, other user agency 1130 supportive outputs, and/or commands to a non-language user agency device 1134. It will be well understood by one of skill in the art that not all components of the disclosed user agency and capability augmentation system 1100 may be used in every application such a system may operate within or may not be used with equal weight. Some applications may make greater use of biosignals 1106 than of context indicating the user history and surroundings. Some applications may necessitate operation completely independent from user input prompt 1140 data. The disclosed user agency and capability augmentation system 1100 may be used in support of such user applications as are described in the embodiments disclosed herein.

FIG. 12 illustrates a biosignals subsystem 1200 in accordance with one embodiment. The biosignals subsystem 1200 may comprise additional biosensors 1202, a biosignals classifier 1204, an electroencephalography or EEG tokenizer 1206, a kinematic tokenizer 1208, and additional tokenizers 1210, each of which may be suitable for one or more streams of biosignal data.

In addition to sensors which may be available on the wearable computing and biosignal sensing device 1104 worn by the user 1102, additional biosensors 1202 may be incorporated into the biosignals subsystem 1200. These may be of a mixture of physical sensors on or near the user's body that connect with network-connected and embedded data sources and models to generate a numerical representation of a biosignal estimate. An appropriate biosignal tokenizer may encode the biosignal estimate with associated data to generate at least one biosignal token sequence. In some embodiments, the mobile or wearable computing and biosignal sensing device 1104 may include a set of sensory peripherals designed to capture user 1102 biometrics. In this manner, the biosignals subsystem 1200 may receive biosignals 1106, which may include at least one of a neurologically sensed signal and a physically sensed signal.

Biosignals 1106 may be tokenized through the use of a biosignals classifier 1204. In some embodiments, these biometric sensors may include some combination of EEG, electrocorticography (ECoG), electrocardiogram (ECG or EKG), electromyography (EMG), electrooculography (EOG), pulse, heart rate variability, blood sugar sensing, dermal conductivity, etc. These biometric data may be converted into a biosignal token sequence in the biosignals classifier 1204, through operation of the EEG tokenizer 1206, kinematic tokenizer 1208, or additional tokenizers 1210, as appropriate.

It is common practice for biosignal raw signal data to be analyzed in real time using a classification system. For EEG signals, a possible choice for an EEG tokenizer 1206 may be canonical correlation analysis (CCA), which ingests multi-channel time series EEG data and outputs a sequence of classifications corresponding to stimuli that the user may be exposed to. However, one skilled in the art will recognize that many other signal classifiers may be chosen that may be better suited to specific stimuli or user contexts. These may include but are not limited to independent component analysis (ICA), xCCA (CCA variants), power spectral density (PSD) thresholding, and machine learning. One skilled in the art will recognize that there are many possible classification techniques. In one example, these signals may consist of SSVEP which occur in response to specific visual stimuli. In other possible embodiments, the classification may consist of a binary true/false sequence corresponding to a P300 or other similar neural characteristic. In some embodiments, there will be a user or stimuli specific calibrated signal used for the analysis. In other embodiments, a generic reference may be chosen. In yet other possible embodiments, the classes may consist of discrete event related potential (ERP) responses. It may be clear to one of ordinary skill in the art that other biosignals including EOG, EMG, and EKG, may be similarly classified and converted into symbol sequences. In other embodiments, the signal data may be directly tokenized using discretization and a codebook. The resulting tokens may be used as part of the biosignals prompt 1136.

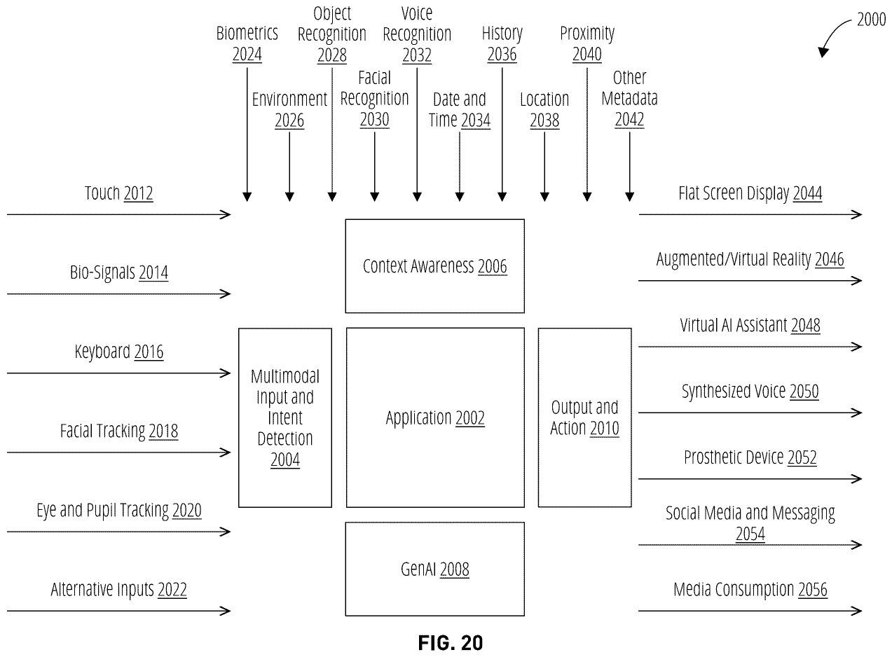

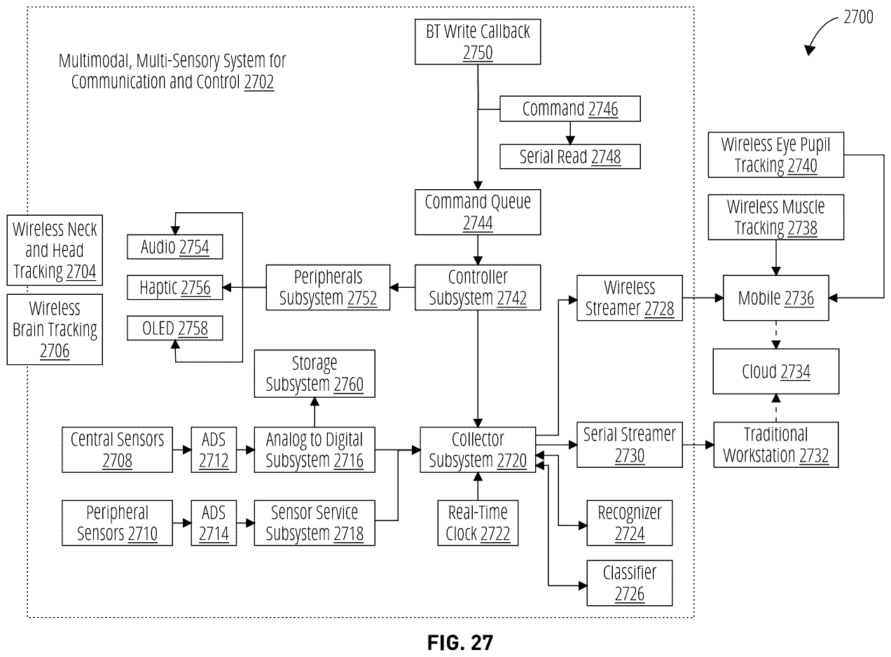

The kinematic tokenizer 1208 may receive biosignals 1106 indicative of user 1102 motion, or motion of some part of a user's body, such as gaze detection based on the orientation and dilation of a user's pupils, through eye and pupil tracking discussed with reference to FIG. 20 or FIG. 27. Such kinematic biosignals 1106 may be tokenized through the operation of the kinematic tokenizer 1208 for inclusion in the biosignals prompt 1136. Additional tokenizers 1210 may operate similarly upon other types of biosignals 1106. In one possible embodiment, the kinematic tokenizer 1208 may utilize a codebook that maps state-space values (position/orientation, velocity/angular velocity) into codes which form the sequence of codes. In other embodiments, a model-based tokenizer may be used to convert motion data into discrete code sequences.

The final output from the biosignals subsystem 1200 may be a sequence of text tokens containing a combination of the token sequences generated from the biosignals 1106, in the form of the biosignals prompt 1136. The biosignals subsystem 1200 may also have a connection with the context subsystem 1300 in advance of any prompt composition. This shared data 1142 connection may bidirectionally inform each of the subsystems to support more precise, or more optimal token generation.

FIG. 13 illustrates a context subsystem 1300 in accordance with one embodiment. The context subsystem 1300 may comprise a raw background material tokenizer 1302, a final background material tokenizer 1304, a raw sensor data tokenizer 1306, a final sensor data tokenizer 1308, a raw device data tokenizer 1310, a final device data tokenizer 1312, a raw application context tokenizer 1314, a final application context tokenizer 1316, and a context prompt composer 1318.

Broadly speaking, the user's context consists of prompts generated from a variety of different data sources, including background material 1108 that provides information about the user's previous history, sensor data 1110 and other device data 1112 captured on or around the user 1102, and application context 1114, i.e., information about the current task or interaction the user 1102 may be engaged in.

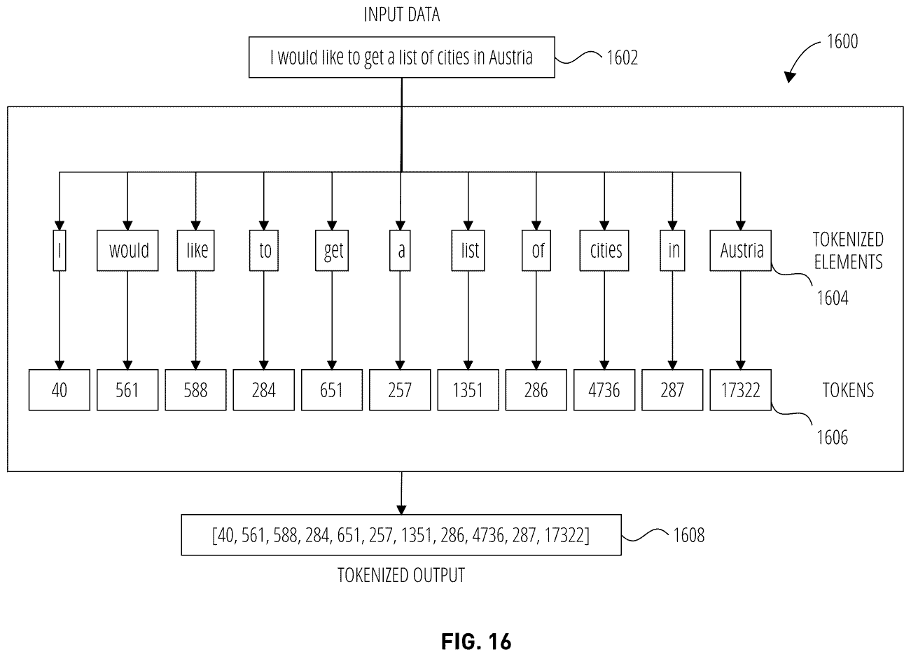

Background material 1108 may be plain text, data from a structured database or cloud data storage (structured or unstructured), or any mixture of these data types. In one embodiment background material 1108 may include textual descriptions of activities that the user 1102 has performed or requested in a similar context and their prior outcomes, if relevant. In one embodiment, background material 1108 may include general information about the user 1102, about topics relevant to the user's current environment, the user's conversational histories, a body of written or other work produced by the user 1102, or notes or other material related to the user's situation which is of a contextual or historical nature. In some embodiments, the background material 1108 may first be converted into a plain text stream and then tokenized using a plaintext tokenizer. This is illustrated in greater detail with respect to FIG. 16.

Sensor data 1110 may include microphone output indicative of sound in the user's environment, temperature, air pressure, and humidity data from climactic sensors, etc., output from motion sensors, and a number of other sensing devices readily available and pertinent to the user's surroundings and desired application of the user agency and capability augmentation system 1100. Other device data 1112 may include camera output, either still or video, indicating visual data available from the user's surrounding environment, location information from a global positioning system device, date and time data, information available via a network based on the user's location, and data from a number of other devices readily available and of use in the desired application of the user agency and capability augmentation system 1100. Scene analysis may be used in conjunction with object recognition to identify objects and people present in the user's environment, which may then be tokenized. The context subsystem may also include a mixture of physical sensors such as microphones and cameras that connect with network-connected and embedded data sources and models to generate a numerical representation of a real-time context estimate.

In some instances, the user 1102 may interact with an application on a computing device, and this interaction may be supported and expanded through the integration of a user agency and capability augmentation system 1100. In these instances, explicit specification of the application may greatly enhance the context subsystem 1300 knowledge of the user 1102 context and may facilitate a more optimal context token set. Application context 1114 data may in such a case be made available to the user agency and capability augmentation system 1100, and data from the application context 1114 data source may be tokenized as part of the operation of the context subsystem 1300, for inclusion in the context prompt 1138. Application context 1114 data may include data about the current application (e.g., web browser, social media, media viewer, etc.) along with the user's interactions associated with the application, such as a user's interaction with a form for an online food order, data from a weather application the user is currently viewing, etc.

For each data source, a raw data tokenizer may generate a set of preliminary tokens 1320. These preliminary tokens 1320 may be passed to final tokenizers for all of the data sources to be consumed as input for the final tokenizers for each data source. Each data source final tokenizer may refine its output based on the preliminary tokens 1320 provided by other data sources. This may be particularly important for background material 1108. For example, the context used by the final background material tokenizer 1304 to determine which background material 1108 elements are likely to be relevant may be the prompt generated by the raw data source tokenizers. For example, camera data and microphone data may indicate the presence and identity of another person within the user's immediate surroundings. Background material 1108 may include emails, text messages, audio recordings, or other records of exchanges between this person and the user, which the final background material tokenizer 1304 may then include and tokenize as of particular interest to the user's present context.

The context subsystem 1300 may send the final tokens output from the final tokenizers for each data source to a context prompt composer 1318. The context prompt composer 1318 may use these final tokens 1322, in whole or in part, to generate a context prompt 1138, which may be the final output from the context subsystem 1300. The context prompt 1138 may be a sequence of text tokens containing the combination of the background, audio/video, and other final tokens 1322 from the final background material tokenizer 1304, final sensor data tokenizer 1308, final device data tokenizer 1312, and final application context tokenizer 1316. In the simplest embodiment, the context prompt composer 1318 concatenates all the final tokens 1322. In other possible embodiments, the context prompt composer 1318 creates as its context prompt 1138 a structured report that includes additional tokens to assist the GenAI in parsing the various final tokens 1322 or prompts.

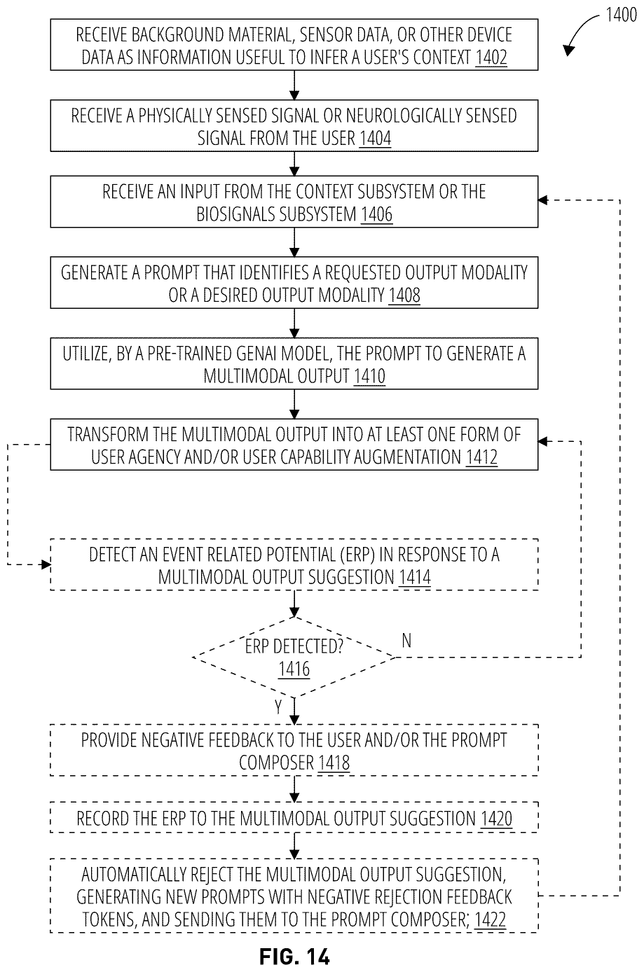

FIG. 14 illustrates an example routine 1400 for implementation using a human augmentation platform using context, biosignals, and Generative AI. Such a routine 1400 may be performed through the operation of a user agency and capability augmentation system 1100 such as that illustrated and described with respect to FIG. 11. Although the example routine 1400 depicts a particular sequence of operations, the sequence may be altered without departing from the scope of the present disclosure. For example, some of the operations depicted may be performed in parallel or in a different sequence that does not materially affect the function of the routine 1400. In other examples, different components of an example device or system that implements the routine 1400 may perform functions at substantially the same time or in a specific sequence.

According to some examples, the method includes receiving, by a context subsystem, at least one of background material, sensor data, and other device data as information useful to infer a user's context at block 1402. For example, the context subsystem 1300 illustrated in FIG. 13 may receive background material, sensor data, or other device data as information useful to infer a user's context. In one embodiment, the at least one of the sensor data and the other device data may be received by the context subsystem from at least one of a camera and a microphone array. In one embodiment, the context subsystem may also receive application context from applications installed on computing or smart devices in communication with the context subsystem.

According to some examples, the method includes receiving, by a biosignals subsystem, at least one of a physically sensed signal and a neurologically sensed signal from the user at block 1404. For example, the biosignals subsystem 1200 illustrated in FIG. 12 may receive a physically sensed signal or neurologically sensed signal from the user as the biosignals 1106 introduced in FIG. 11. In one embodiment, the biosignals subsystem may receive biosignals data from biometric sensors for at least one of electroencephalography (EEG), electrocorticography (ECoG), electrocardiogram (ECG or EKG), electromyography (EMG), electrooculography (EOG), pulse determination, heart rate variability determination, blood sugar sensing, and dermal conductivity determination.

According to some examples, the method includes receiving, by a prompt composer, an input from at least one of the context subsystem and the biosignals subsystems; at block 1406. For example, the prompt composer 1116 illustrated in FIG. 11 may receive an input from the context subsystem or the biosignals subsystem. In one embodiment, the prompt composer may additionally receive a user input prompt. In this manner, a user may directly and explicitly provide instruction to the prompt composer.

According to some examples, the method includes generating, by the prompt composer, a prompt that identifies at least one of a requested output modality and a desired output modality at block 1408. For example, the prompt composer 1116 illustrated in FIG. 11 may generate a prompt that identifies a requested output modality or a desired output modality. In one embodiment, the prompt composer may generate at least one of a single token, a string of tokens, a series of conditional or unconditional commands suitable to prompt the GenAI model, tokens that identify at least one of the requested output modality and the desired output modality, an embedding to be provided separately to the GenAI model for use in an intermediate layer of the GenAI model, and multiple tokenized sequences at once that constitute a series of conditional commands.

According to some examples, the method includes utilizing, by a pre-trained Generative Artificial Intelligence (GenAI) model, the prompt to generate a multimodal output at block 1410. For example, the GenAI 1118 illustrated in FIG. 11 may utilize, by a pre-trained GenAI model, the prompt to generate a multimodal output. In one embodiment, the GenAI model may be at least one of LLMs, Generative Pre-trained Transformer (GPT) models, text-to-image creators, visual art creators, and generalist agent models.

According to some examples, the method includes transforming, by an output stage, the multimodal output into at least one form of user agency, user capability augmentation, and combinations thereof at block 1412. For example, the multimodal output stage 1120 illustrated in FIG. 11 may transform the multimodal output into at least one form of user agency and/or user capability augmentation. In one embodiment, the at least one form of the user agency includes neural stimulation to the user with Transcranial Direct Current Stimulation (tDCS), direct brain stimulation (DBS), or other known neural stimulation techniques. In one embodiment, the multimodal output may be in the form of at least one of text-to-speech utterances, written text, multimodal artifacts, other user agency supportive outputs, and commands to a non-language user agency device. In one embodiment, the output stage may receive an output mode selection signal from the user. The output mode selection signal may be a direct selection or may be generated from the user's biosignals. The output mode selection signal may instruct the output stage of a choice between multimodal outputs or may direct one or more alternative multimodal outputs to alternate endpoints. In one embodiment, an encoder/parser framework, such as the encoder/parser 122 of FIG. 11, may encode the multimodal output to provide control commands to control at least one of a non-language user agency device, a robot system, and smart AI-powered devices.

According to some examples, the method includes detecting, using an output adequacy feedback system, an event related potential (ERP) which may be an error-related negativity in response to a multimodal output suggestion at block 1414.

According to some examples, the method includes, if an ERP is detected at decision block 1416, providing negative feedback to at least one of the user and the prompt composer at block 1418. The prompt composer may provide the negative feedback to the GenAI model.

According to some examples, the method includes, if an ERP is detected at decision block 1416, recording the ERP to the multimodal output suggestion at block 1420.

According to some examples, the method includes, if an ERP is detected at decision block 1416, automatically rejecting the multimodal output suggestion, generating new prompts with negative rejection feedback tokens, and sending the negative rejection feedback tokens to the prompt composer at block 1422.

### Simultaneous Users

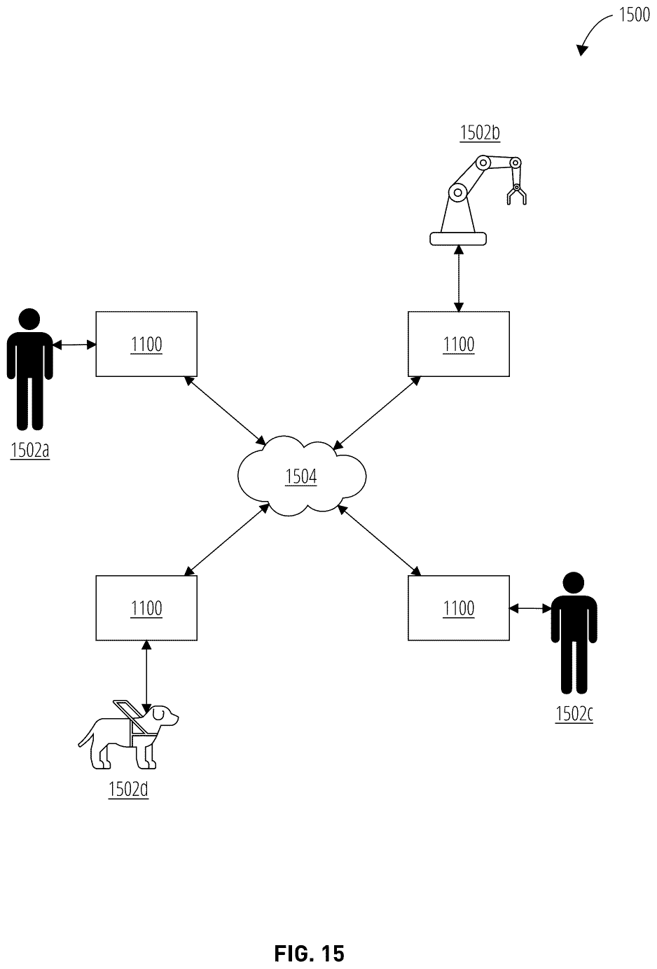

FIG. 15 illustrates a multiuser system 1500 in accordance with one embodiment. In some embodiments more than one user may be interacting simultaneously with a common artifact, environment, or in a social scenario. In these embodiments, each of simultaneous user 1502a, 1502b, 1502c, and 1502d, may interact with an instance of one or more of the user agency and capability augmentation system 1100 embodiments described herein.

Further, when multiple such user agency and capability augmentation systems 1100 are present, they may establish direct, digital communication with each other via a local area or mesh network 1504 to support direct context transmission and exchange of GenAI 1118 outputs.

In some instances, one or more of the simultaneous users may be a robot or other autonomous agent, such as simultaneous user 1502b. In other instances, one or more of the simultaneous users may be another human user, as shown by simultaneous user 1502c. In yet other instances, one or more of the users may be an assistive animal such as a sight impairment support dog, as shown by simultaneous user 1502d.

FIG. 16 illustrates an exemplary tokenizer 1600 in accordance with one embodiment. Input data 1602 may be provided to the exemplary tokenizer 1600 in the form of a text string typed by a user. In one embodiment, the text string may be generated by performing voice-to-text conversion on an audio stream. The exemplary tokenizer 1600 may detect tokenizable elements 1604 within the input data 1602. Each tokenizable element 1604 may be converted into a token 1606. The set of tokens 1606 created from the tokenizable elements 1604 of the input data 1602 may be sent from the exemplary tokenizer 1600 as tokenized output 1608. The set of tokens 1606 may be such as are used to create the prompt 1144 of FIG. 11.

For structured historical data such as plaintext, database, or web-based textual content, tokens may consist of the numerical indexes in an embedded or vectorized (e.g., word2vec or similar) representation of the text content such as are shown here. In some embodiments, a machine learning technique called an autoencoder may be utilized to transform plaintext inputs into high dimensional vectors that are suitable for indexing and tokenization ingestion by the prompt composer 1116 introduced with respect to FIG. 11.

In some embodiments, data to be tokenized may include audio, visual, or other multimodal data. For images, video, and similar visual data, tokenization may be performed using a convolution-based tokenizer such as a vision transformer. In some alternate embodiments, multimodal data may be quantized and converted into tokens 1606 using a codebook. In yet other alternate embodiments, multimodal data may be directly encoded and for presentation to a language model as a vector space encoding. An exemplary system that utilizes this tokenizer strategy is Gato, a generalist agent capable of ingesting a mixture of discrete and continuous inputs, images, and text as tokens.

FIG. 17A illustrates an isometric view of a BCI headset system 1700 in accordance with one embodiment. The BCI headset system 1700 comprises an augmented reality display lens 1702, a top cover 1704, an adjustable strap 1706, a padding 1708, a ground/reference electrode 1710, a ground/reference electrode adjustment dial 1712, a biosensor electrodes 1714, a battery cell 1716, a fit adjustment dial 1718, and a control panel cover 1720.

The augmented reality display lens 1702 may be removable from the top cover 1704 as illustrated in FIG. 17C. The augmented reality display lens 1702 and top cover 1704 may have magnetic portions that facilitate removably securing the augmented reality display lens 1702 to the top cover 1704. The augmented reality display lens 1702 may in one embodiment incorporate a frame around the lens material allowing the augmented reality display lens 1702 to be handled without depositing oils on the lens material.

The adjustable strap 1706 may secure the BCI headset system 1700 to a wearer's head. The adjustable strap 1706 may also provide a conduit for connections between the forward housing 1732 shown in FIG. 17C and the components located along the adjustable strap 1706 and to the rear of the BCI headset system 1700. Padding 1708 may be located at the front and rear of the BCI headset system 1700, as well as along the sides of the adjustable strap 1706, as illustrated. A fit adjustment dial 1718 at the rear of the BCI headset system 1700 may be used to tighten and loosen the fit of the BCI headset system 1700 by allowing adjustment to the adjustable strap 1706.

A snug fit of the BCI headset system 1700 may facilitate accurate readings from the ground/reference electrodes 1710 at the sides of the BCI headset system 1700, as illustrated here in FIG. 17A as well as in FIG. 17C. A snug fit may also facilitate accurate readings from the biosensor electrodes 1714 positioned at the back of the BCI headset system 1700. Further adjustment to these sensors may be made using the ground/reference electrode adjustment dials 1712 shown, as well as the biosensor electrode adjustment dials 1724 illustrated in FIG. 17B.

In addition to the padding 1708, biosensor electrodes 1714, and fit adjustment dial 1718 already described, the rear of the BCI headset system 1700 may incorporate a battery cell 1716, such as a rechargeable lithium battery pack. A control panel cover 1720 may protect additional features when installed, those features being further discussed with respect to FIG. 17B.

FIG. 17B illustrates a rear view of a BCI headset system 1700 in accordance with one embodiment. The control panel cover 1720 introduced in FIG. 17B is not shown in this figure, so that underlying elements may be illustrated. The BCI headset system 1700 further comprises a control panel 1722, a biosensor electrode adjustment dials 1724, an auxiliary electrode ports 1726, and a power switch 1728.

With the control panel cover 1720 removed, the wearer may access a control panel 1722 at the rear of the BCI headset system 1700. The control panel 1722 may include biosensor electrode adjustment dials 1724, which may be used to calibrate and adjust settings for the biosensor electrodes 1714 shown in FIG. 17A.

The control panel 1722 may also include auxiliary electrode ports 1726, such that additional electrodes may be connected to the BCI headset system 1700. For example, a set of gloves containing electrodes may be configured to interface with the BCI headset system 1700, and readings from the electrodes in the gloves may be sent to the BCI headset system 1700 wirelessly, or via a wired connection to the auxiliary electrode ports 1726.

The control panel 1722 may comprise a power switch 1728, allowing the wearer to power the unit on and off while the control panel cover 1720 is removed. Replacing the control panel cover 1720 may then protect the biosensor electrode adjustment dials 1724 and power switch 1728 from being accidentally contacted during use. In one embodiment, a power LED may be incorporated onto or near the power switch 1728 as an indicator of the status of unit power, e.g., on, off, battery low, etc.

FIG. 17C illustrates an exploded view of a BCI headset system 1700 in accordance with one embodiment. The BCI headset system 1700 further comprises a universal serial bus or USB port 1730 in the rear of the BCI headset system 1700 as well as a forward housing 1732 which may be capable of holding a smart phone 1734. The USB port 1730 may in one embodiment be a port for a different signal and power connection type. The USB port 1730 may facilitate charging of the battery cell 1716 and may allow data transfer through connection to additional devices and electrodes.

The top cover 1704 may be removed from the forward housing 1732 as shown to allow access to the forward housing 1732, in order to seat and unseat a smart phone 1734. The smart phone 1734 may act as all or part of the augmented reality display. In a BCI headset system 1700 incorporating a smart phone 1734 in this manner, the augmented reality display lens 1702 may provide a reflective surface such that a wearer is able to see at least one of the smart phone 1734 display and the wearer's surroundings within their field of vision.

The top cover 1704 may incorporate a magnetized portion securing it to the forward housing 1732, as well as a magnetized lens reception area, such that the augmented reality display lens 1702 may, through incorporation of a magnetized frame, be secured in the front of the top cover 1704, and the augmented reality display lens 1702 may also be removable in order to facilitate secure storage or access to the forward housing 1732.

FIG. 17D illustrates an exploded view of a BCI headset system 1700 in accordance with one embodiment. The BCI headset system 1700 further comprises a smart phone slot 1736 in the forward housing 1732. When the augmented reality display lens 1702 and top cover 1704 are removed to expose the forward housing 1732 as shown, the smart phone slot 1736 may be accessed to allow a smart phone 1734 (not shown in this figure) to be inserted.

### Additional System Embodiment Details

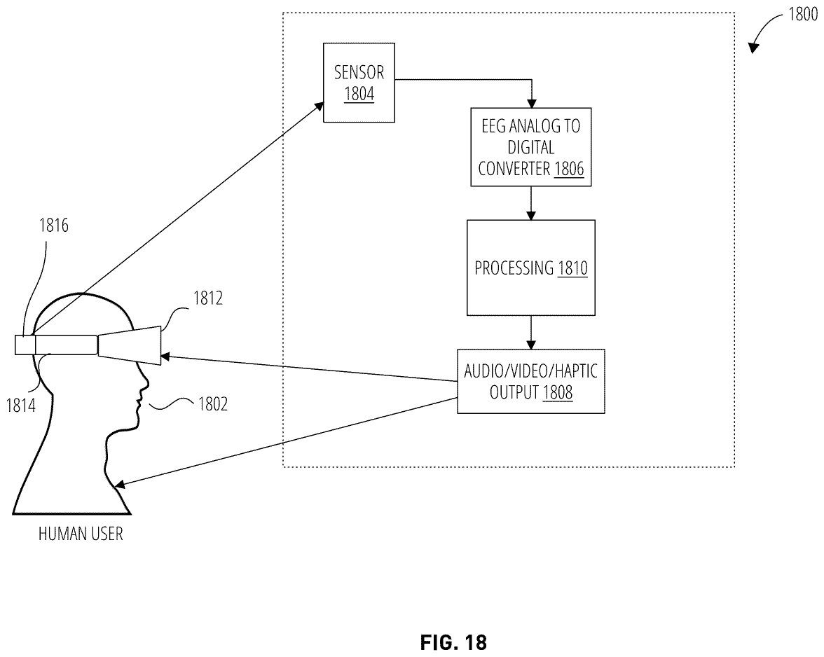

FIG. 18 illustrates an embodiment of a BCI+AR environment 1800. The BCI+AR environment 1800 comprises a sensor 1804, an EEG analog to digital converter 1806, an Audio/Video/Haptic Output 1808, a processing 1810, a strap 1814, an augmented reality glasses 1812, a human user 1802, and a BCI 1816. A human user 1802 is wearing BCI 1816, which is part of a headset. When the human user 1802 interacts with the environment, the sensor 1804, located within the BCI 1816, reads the intentions and triggers the operating system. The EEG analog to digital converter 1806 receives the sensor 1804 output (e.g., intention). EEG analog to digital converter 1806 transforms the sensor output into a digital signal which is sent to processing 1810. The signal is then processed, analyzed and mapped to an Audio/Video/Haptic Output 1808 and displayed on the augmented reality glasses 1812.

In an embodiment, strap 1814 is a head strap for securing the AR+BCI to the human head. In some embodiments, such as an implantable BCI, and AR system, the strap may not be used. The strapless system may use smart glasses or contact lenses. There may be multiple sensors, but no less than one sensor, in different embodiments. After seeing the output, the user may have different bio-signals from the brain, and as such this is a closed-loop biofeedback system. As the user focuses more on the SSVEP stimuli, the audio may feedback by frequency, power (volume), and selected cue audio to assist the human in reinforcing their focus on the stimuli. This may also occur with the vibration type and intensity of the haptics, as well as additional peripheral visual cues in the display. This feedback is independent from the audio and haptics that may play back through the AR headset via a smartphone. It is even possible to remotely add to the sensory mix that of olfactory (smell) feedback that actually travels through entirely different parts of the brain that has been shown to be one of the strongest bio-feedback reinforcements in human cognitive training.

As a non-limiting example, when someone uses the BCI for the first time, they are considered a “naïve” user, or one whose brain has never been trained with this kind of user interface. As a user continues to use it, their brain becomes less naïve and more capable and trained. They may become quicker and quicker at doing it. This is reinforcement learning—the BCI allows someone to align their intention and attention to an object and click it.

In an embodiment, to enrich the user interface experience, multiple feedback modalities (auditory, visual, haptic, and olfactory) may be available for choosing the most advantageous feedback modality for the individual or for the type of training. For example, when an appropriate brain wave frequency is generated by the user, real-time feedback about the strength of this signal may be represented by adjusting the intensity and frequency of the audio or haptic feedback. In addition, the possibility of using multimodal feedback may allow multiple sensory brain regions to be stimulated simultaneously, which enhances the neural signal and representation of feedback, thereby accelerating learning and neural plasticity.

An advantage of using odors as reinforcers may be due to the direct link between the brain areas that sense smell (olfactory cortex) and those that form memories (hippocampus) and produce emotions (amygdala). Odors may strengthen memory encoding, consolidation, and trigger recall.

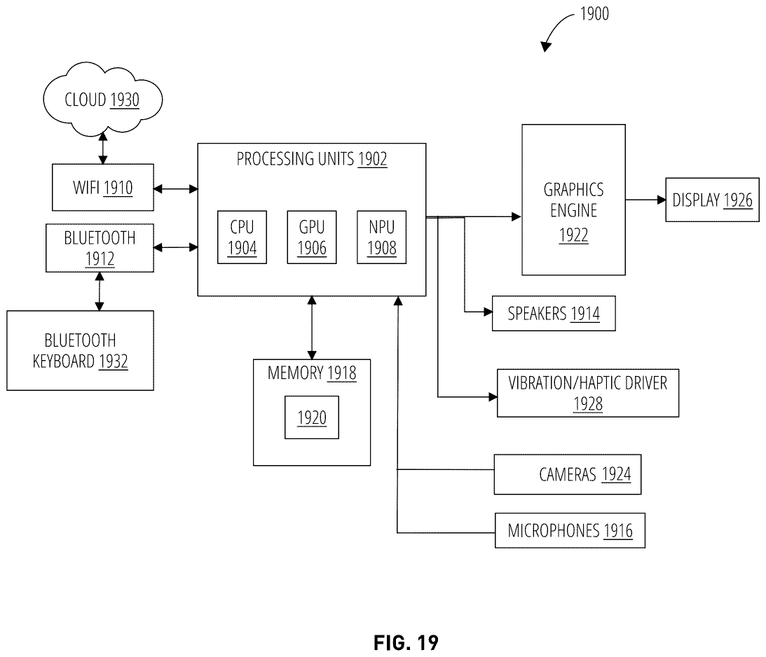

FIG. 19 illustrates components of an exemplary augmented reality device logic 1900. The augmented reality device logic 1900 comprises a graphics engine 1922, a camera 1924, processing units 1902, including one or more central processing units CPU 1904, graphical processing units GPU 1906, and/or neural processing units NPU 1908, a WiFi 1910 wireless interface, a Bluetooth 1912 wireless interface, speakers 1914, microphones 1916, one or more memory 1918, logic 1920, a visual display 1926, and vibration/haptic driver 1928.

The processing units 1902 may in some cases comprise programmable devices such as bespoke processing units optimized for a particular function, such as AR related functions. The augmented reality device logic 1900 may comprise other components that are not shown, such as dedicated depth sensors, additional interfaces, etc.

Some or all of the components in FIG. 19 may be housed in an AR headset. In some embodiments, some of these components may be housed in a separate housing connected or in wireless communication with the components of the AR headset. For example, a separate housing for some components may be designed to be worn or a belt or to fit in the wearer's pocket, or one or more of the components may be housed in a separate computer device (smartphone, tablet, laptop or desktop computer etc.) which communicates wirelessly with the display and camera apparatus in the AR headset, whereby the headset and separate device constitute the full augmented reality device logic 1900. A user may also communicate with the AR headset via a Bluetooth keyboard 1932. Additionally, the AR headset may communicate with the cloud 1930 via WiFi 1910 or cellular connection.

The memory 1918 comprises logic 1920 to be applied to the processing units 1902 to execute. In some cases, different parts of the logic 1920 may be executed by different components of the processing units 1902. The logic 1920 typically comprises code of an operating system, as well as code of one or more applications configured to run on the operating system to carry out aspects of the processes disclosed herein. In one embodiment, the memory 1918 and logic 1920, or a similarly integrated or peripherally connected storage device, may include a non-transitory computer-readable medium storing instructions that, when executed by a processing system coupled to a structural chassis of a head-mounted device, cause the processing system to detect one or more sensor modules connected to the structural chassis via a standardized electromechanical mating interface, identify, from a module-identification signal received via the standardized interface, a module type and a capability profile for each detected sensor module, construct a sensor configuration representation based on the identified sensor modules, compute, for each channel of each detected sensor module, a confidence measure based on at least one of electrical impedance, signal variance, power-line interference, and contact-quality metric, and adjust a weight assigned to each channel in a signal-processing pipeline based on the computed confidence measure, thereby improving signal-to-noise ratio of biosignals acquired by the head-mounted device.

FIG. 20 is a block diagram of nonverbal multi-input and feedback device 2000 of a nonverbal multi-input and feedback device such as herein. It may be a block diagram of a portion of the device such as a processing portion of the device. FIG. 20 may be a high-level system architecture block diagram that helps explain the major building blocks. Block diagram of nonverbal multi-input and feedback device 2000 may be applied to the overall system (e.g., multiple devices used as inputs), into a common universal application interface that allows the application 2002 to synchronize data coming from multiple devices and process signals with meta data, plus vocabulary and output logic to a plurality of output methods.

In the center of block diagram of nonverbal multi-input and feedback device 2000 is the application 2002 or main processing block. To the left is the multimodal input and intent detection 2004 block which receives and processes user inputs from sensors (e.g., based on user input received by the sensors) such as touch 2012; bio-signals 2014; keyboard 2016; facial tracking 2018; eye and pupil tracking 2020; and alternative inputs 2022. This multimodal input and intent detection 2004 block feeds the processing from these inputs to the application 2002.

Above is a context awareness 2006 block which receives and processes metadata inputs from sensors such as biometrics 2024; environment 2026; object recognition 2028; facial recognition 2030; voice recognition 2032; date and time 2034; history 2036; location 2038; proximity 2040; and other metadata 2042 inputs. This context awareness 2006 block feeds the processing from these inputs to the application 2002.

To the right is an output and action 2010 block which sends outputs to displays, computing devices, controllers, speakers and network communication devices such as flat screen flat screen display 2044; augmented/virtual reality 2046; virtual AI assistant 2048; synthesized voice 2050; prosthetic device 2052; social media and messaging 2054; media consumption 2056; and other output. The outputs may include control commands and communication sent to other computing devices. They may include text, graphics, emoji, and/or audio.

Below is a GenAI 2008 block that provides a lexicon or vocabulary in the selected language to the application. FIG. 20 may also be applied to a single sensory device unto itself. This may be a “Big Idea” in so far as the architecture may scale from a single closed-loop system as well as combinations of sensory I/O devices. It may be a system of systems that scale up, down and play together.

The system in block diagram of nonverbal multi-input and feedback device 2000 comprises one (or more) sensory input, one intent detection application programming interface (API), one application, one (or more) meta data, one (or more) vocabulary, one (or more) output and action method, and one (or more) output/actuation system or device. It may be thought of as a universal “augmented intelligence” engine that takes inputs, enriches them with extra meaning, and directs the output based on instructions for the enriched information.

In a simple embodiment of diagram, a user sees a symbol or button indicating “help”, and presses it, and the device says “help”. In a more complicated embodiment of block diagram of nonverbal multi-input and feedback device 2000, a user sees a symbol or button indicating “help” and presses it. Here, rather than the device saying “help,” it learns that the user is connected to a caregiver with logic to send urgent matters to that person via text or instant message when away from home. The device may geolocation data that indicates the user is away from home; tag the communication with appended contextual information; and its output and action logic tell the system to send a text message to the caregiver with the user's location in a human-understandable grammatically correct phrase “Help, I'm in Oak Park” including the user's Sender ID/Profile and coordinates pinned on a map.

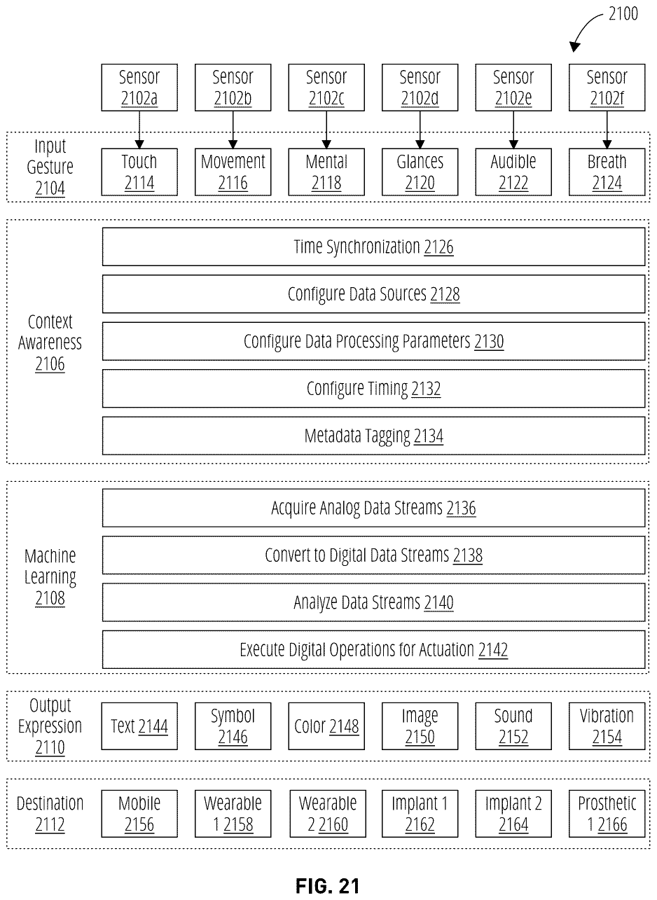

FIG. 21 is a block diagram of a single framework of a nonverbal multi-input and feedback device 2100 such as herein. The block diagram of a single framework of a nonverbal multi-input and feedback device 2100 may be of a single framework for translating diverse sensor inputs into a variety of understandable communication and command outputs for a nonverbal multi-input and feedback device such as herein. The single framework of a nonverbal multi-input and feedback device comprises sensors 2102a-2102f, input gestures 2104, context awareness 2106, machine learning 2108, output expressions 2110, and destinations 2112. Input gestures 2104 may include touch 2114, movement 2116, mental 2118, glances 2120, audible 2122, and breath 2124. Context awareness 2106 may include time synchronization 2126, configure data sources 2128, configure data processing parameters 2130, configure timing 2132, and metadata tagging 2134. Machine learning 2108 may include an acquire analog data streams 2136, convert to digital data streams 2138, analyze data streams 2140, and execute digital operations for actuation 2142. Output expressions 2110 may include text 2144, symbol 2146, color 2148, an image 2150, sound 2152, and vibration 2154. Destinations 2112 may include a mobile 2156, a wearable 1 2158, a wearable 2 2160, an implant 1 2162, an implant 2 2164, and a prosthetic 1 2166.

FIG. 21 may describe in more detail what kind of processing is happening within and across the blocks of FIG. 20. Specifically, the left intention signals being combined with context awareness metadata to enrich the data in order to determine the logic of the output and action. FIG. 21 may include the description of the GenAI 2008 and application 2002 boxes of FIG. 20, though not shown. It may be a block diagram of a portion of the device such as a processing portion of the device. In the framework, input from the sensors 2102a-2102f (e.g., due to input received by the sensors) are received by or as an input gesture 2104. In the framework, context awareness 2106 awareness is used to interpret or determine the user gesture or intent from the inputs received. In the framework machine learning 2108 is used to interpret or determine the user gesture or intent from the inputs received. In the framework, output expression 2110 is used to determine the outputs, such as control commands and communication sent to other computing devices that include text, graphics, emoji, and/or audio. In the framework, destination 2112 is used to determine where the outputs are sent, such as to what other computing devices the command and/or communications are to be sent (such as by the network). The user's Primary and Secondary language preferences are accessed during the processing of intention data which is stored in the GenAI 2008 subsystem such as shown in FIG. 20, and may be accessed in the context awareness 2106, machine learning 2108 and output and action 2010 systems and methods in FIG. 20 and FIG. 21.

FIG. 22 illustrates a block diagram of nonverbal multi-input and feedback device 2200 in one embodiment. The block diagram of nonverbal multi-input and feedback device 2200 shows a system comprising analog input 2202, sensors 2204, processing 2206, digital output 2208, and output methods 2210 that may be performed with the digital output 2208.

The system illustrated may include an application programming interface (API) that is interoperable with multiple types of analog input 2202 from the sensors 2204. The system illustrated may also comprise a real-time clock for tracking, synchronizing, and metadata 2220 tagging of data streams and analog inputs 2202. The system further comprises a subsystem for data storage and management, for historical data 2212 in some embodiments. The system may comprise a subsystem for personalization settings 2218, as well as a subsystem for sourcing and integrating metadata 2220 into the application 2222 and data stream. The system may further comprise a software application 2222. In some embodiments, the system may include a graphical user interface (GUI) for the software application for the user. In other embodiments, the system may include a GUI for the software application for others who are connected to a system user.

A subsystem of the system may include processing for visual 2226, audible 2228, and written 2230 languages. This language subsystem may differentiate between the user's primary and secondary languages 2224. The language subsystem may set the secondary language manually or automatically. Attributes processed by visual 2226, audible 2228, and written 2230 language subsystems may include but not be limited to color, image, graphics, audible tones, phonemes, dialects, jargon, semantics, tonality, and written characters. In one embodiment, the language subsystems may consist of a suitably trained generative AI model.

The system may include a subsystem of digital outputs 2208 and output methods 2210, that may be configured either manually or automatically. The variety of output methods 2210 may include a network 2216 interface connection. The system may comprise a subsystem for managing data transfer over the network 2216.

The system in some embodiments may comprise a historical data 2212 subsystem for closed-loop machine learning of the system and subsystems and the sensory devices being used with the system. In some embodiments, improved models, algorithms and software may be pushed from the learning system 2214 to update and be used within the system and subsystems and the sensory devices being used with the system.

In one embodiment, the system and subsystems may operate entirely on a sensory device. In one embodiment, the system and subsystems may operate partially on a sensory device and partially distributed to other devices or the cloud. In one embodiment, the system and subsystems may operate entirely distributed on other devices or the cloud.

The system of FIG. 22 may be one embodiment of a fully self-contained brain computer interface in a wireless headset, comprising an augmented reality display as part of the digital output 2208, at least two sensors 2204 for reading a bio-signal from a user as analog input 2202, at least one processing 2206 module for the augmented reality display, at least one biofeedback device that produces at least one of a visual, audible, and tactile effect in communication with the processing module to provide feedback to the user, a wireless network interface that transmits and receives data to and from other devices over the processing 2206, wherein the data is at least one of stored, passed through, and processed on the fully self-contained BCI, as part of the output methods 2210, a battery, wherein the battery provides power to one or more of the augmented reality display, the at least two sensors, the processing module, and the at least one biofeedback device, at least one of onboard storage or remote storage with enough memory to store, process and retrieve the data, and a printed circuit board.

Bio-signals from the user may comprise at least one of EEG, ECG, functional near infrared spectroscopy (fNIRS), Magnetoencephalography (MEG), EMG, EOG, and Time-Domain variants (TD-) of these bio-signal processing methods. Bio-signals may also comprise a visually evoked potential, an audio evoked potential, a haptic evoked potential, and a motion evoked potential, and other bio-signals from multiple sources attached to other body parts other than a user's head.

The at least one processing module for the augmented reality display may include a processor that renders a stimulation effect. This stimulation effect may be at least one of a timed visual stimulation on the augmented reality display, a timed audio stimulation, and a haptic stimulation on the fully self-contained BCI configured to evoke a measurable response in a user's brain. The processing module may include a processor that analyzes and maps the bio-signal into a digital command. This digital command may include at least one of instructions for a visual output configured for displaying on the augmented reality display and instructions for triggering a visual effect. The processing module may be embodied as the processing units 1902 introduced in FIG. 19.

The printed circuit board may include at least one of the at least two sensors, the processing module, the at least one biofeedback device, the battery, and combinations thereof. The printed circuit board may be configured to emulate a Bluetooth keyboard and send output data to at least one of a mobile device, a computer, and the augmented reality display. The output data may include at least one of a letter, a character, a number, and combinations thereof.

Processing performed by the processing module may include the visually evoked potential, the audio evoked potential, and the haptic evoked potential. The bio-signal is processed and analyzed in real-time. The processing module may have different modes, including raw, simmer, and cooked modes, a human interface device-keyboard mode, and combinations thereof. The system may also have a strapless mode, wherein the fully self-contained BCI uses smart glasses or smart contact lenses, an implantable brain computer interface, and an AR system.

The raw mode may stream a full EEG sensor stream of data for further processing locally on device or remotely in a cloud via a mobile or desktop internet connected device that may filter, recognize, or interact with the full EEG sensor stream of data. The cooked mode may comprise a fully processed custom digital command generated by a local recognizer and classifier. This cooked mode data may consist of a sequence of biosignals tokens, as provided by the EEG tokenizer 1206, the kinematic tokenizer 1208, or the additional tokenizers 1210 introduced with respect to FIG. 12.The fully processed custom digital command may be sent to a destination system over the network 2216, per the “send it” output method 2210, and executed on the destination system, with no raw data passed to the user. The recognizer and classifier may be embodied as the recognizer 2724 and classifier 2726 introduced in FIG. 27. The simmer mode may be a hybrid combination between the raw mode and the cooked mode, and the at least one processing module may intersperse a raw data stream with cooked metadata 2220 appended to bio-signal data.

Time domain data may be appended to raw data, cooked data, and simmer data in order for the system to process bio-signal data streams from multiple bio-signal data sources and ensure all bio-signal data streams are synchronized. Metadata from other sensors and data sources may be appended to the raw data, the cooked data, and the simmer data in order for a classifier to alter the command that is sent to execute on a destination system. This classifier may be embodied as the classifier 2726 introduced in FIG. 27. Visual, audible, and tactile sensory frequency stimulators may be appended with metadata from other sensors 2204 and data sources wherein the visual, audible, and tactile sensory frequency stimulators are altered to produce a unique pattern which includes metadata that is decodable by the recognizer and classifier.

The fully self-contained BCI may be electrically detached from the augmented reality display and may be configured to transfer data wirelessly or via a wired connection to an external augmented reality display. The fully self-contained BCI in the wireless headset may be an accessory apparatus that is configured to be temporarily mechanically integrated with another wearable device and configured to transfer data wirelessly or via a wired connection to the other wearable device. The fully self-contained BCI may in another embodiment be permanently mechanically integrated with another wearable device and may transfer data wirelessly or via a wired connection to the other wearable device.

A charging port may be connected to a charging bridge, wherein the charging bridge includes internal circuitry and data management connected to the fully self-contained BCI and the augmented reality display. The internal circuitry may include charging circuitry, thereby allowing charging of both the fully self-contained BCI and the augmented reality display with the charging circuitry.

The fully self-contained BCI may be configured to generate visual, auditory, or haptic stimulations to a user's visual cortex, a user's auditory cortex, and a user's somatosensory cortex, thereby resulting in detectable brain wave frequency potentials that are at least one of stimulated, event-related, and volitionally evoked. The BCI may process the detectable brain wave frequencies, thereby facilitating mapping of bio-signals to digital commands. Stimulation effects and digital commands may be altered with metadata from other sensors or data sources.

The BCI may synchronize bio-signal processing from multiple sensors with a real-time clock such as the real-time clock 2722 introduced in FIG. 27. Digital commands may be associated with a device. The device may be operated according to the digital commands. The BCI may stimulate the user's visual cortex, wherein stimulating includes biofeedback to the user's visual cortex and biofeedback confirmation of the operating of the device. The BCI may stimulate the user's somatosensory cortex, wherein stimulating includes the biofeedback confirmation of the operating of the device. The BCI may stimulate the user's auditory cortex, wherein the stimulating includes biofeedback confirmation of the operating of the device.

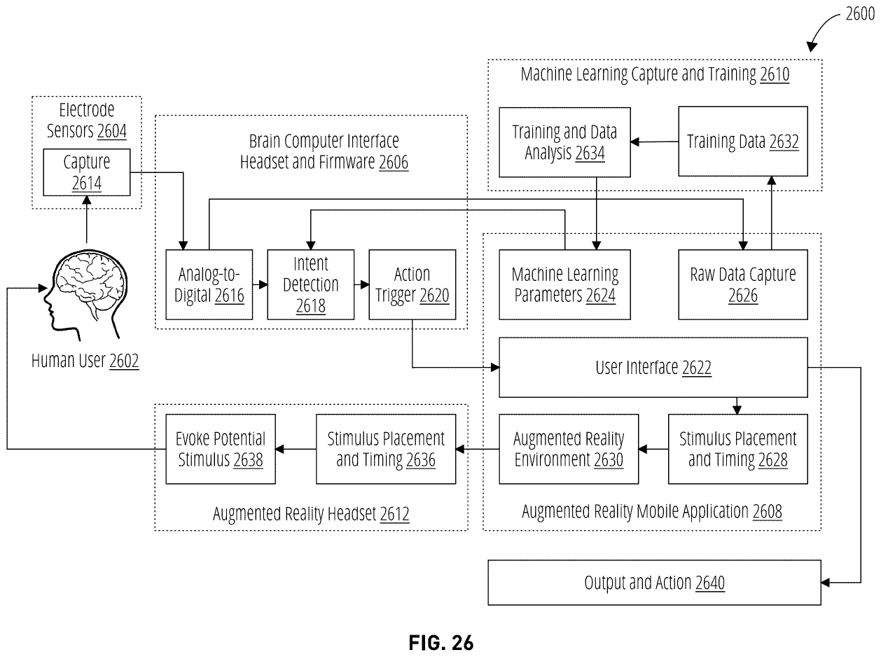

The fully self-contained BCI may be configured to utilize AI machine learning for pattern recognition, classification, and personalization that operates while the fully self-contained BCI is not connected to a network 2216. The AI machine learning may be embodied as the machine learning 2108 introduced in FIG. 21. It may be included in the learning system 2214 of this figure. It may also be supported by the machine learning capture and training 2610 and machine learning parameters 2624 introduced in FIG. 26. The AI machine learning may act as one or more of an auto-tuning dynamic noise reducer, a feature extractor, and a recognizer-categorizer-classifier. AI machine learning training may be applied when the fully self-contained BCI is connected to the network 2216 to create an individualized recognizer-categorizer-classifier. Derived outputs of the AI machine learning training may be stored in a GenAI model in cloud storage or on a mobile computing device having at least one of a wireless connection and a wired connection to the wireless headset and being at least one of mounted on the wireless headset and within wireless network range of the wireless headset. Synthesized insights derived from the AI machine learning and the GenAI may be stored in cloud storage or on the mobile computing device and may be used to generate an individualized executable recognizer-categorizer-classifier downloadable onto the at least one processing 2206 module of the fully self-contained BCI or the mobile computing device via at least one of a wireless connection and a wired connection between the network and a BCI storage device for offline usage without network dependencies. The system may be configured to interface with resource constrained devices including wearable devices, implantable devices, and internet of things (IoT) devices. At least one biofeedback device may be configured to stimulate at least one of a user's central nervous system and peripheral nervous system.

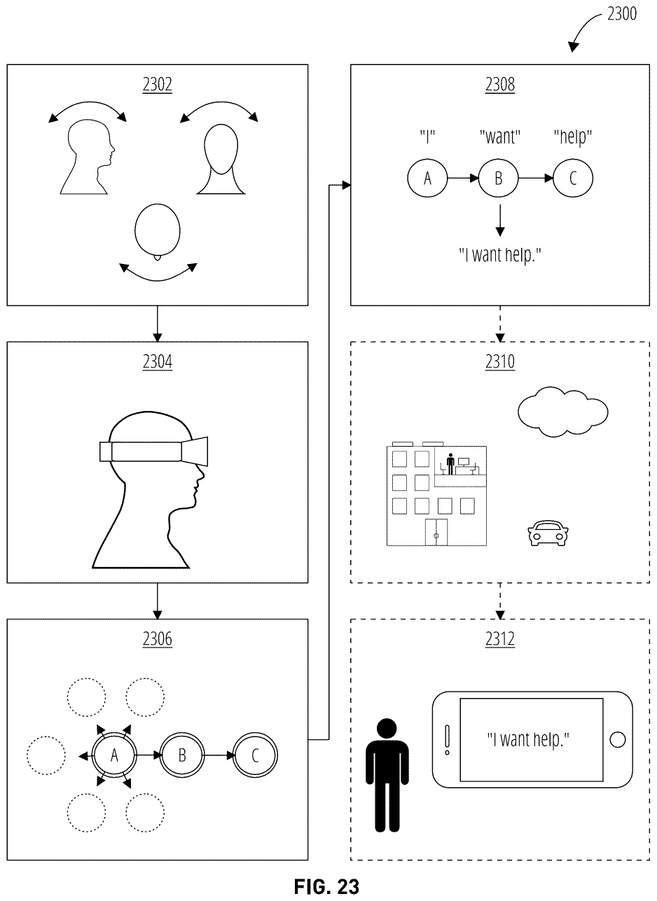

FIG. 23 illustrates a logical diagram of a user wearing an augmented reality headset 2300 that includes a display, speakers and vibration haptic motors and an accelerometer/gyroscope and magnetometer. FIG. 23 shows the flow of activity from head motion analog input 2302 as captured by a headset with head motion detection sensors 2304, through how a user selects options through head motion 2306 and the application creates output based on the user's selected options 2308. On the condition that system detects the user is away from home 2310, FIG. 23 shows that the system may send output to a caregiver via text message 2312.

The user may calibrate the headset based on the most comfortable and stable neck and head position which establishes the X/Y/Z position of 0/0/0. Based on this central ideal position, the user interface is adjusted to conform to the user's individual range of motion, with an emphasis of reducing the amount of effort and distance needed to move a virtual pointer in augmented reality from the 0/0/0 position to outer limits of their field of view and range of motion. The system may be personalized with various ergonomic settings to offset and enhance the user's ease of use and comfort using the system. A head motion analog input 2302 may be processed as analog streaming data and acquired by the headset with head motion detection sensors 2304 in real-time, and digitally processed, either directly on the sensory device or via a remotely connected subsystem. The system may include embedded software on the sensory device that handles the pre-processing of the analog signal. The system may include embedded software that handles the digitization and post-processing of the signals. Post-processing may include but not be limited to various models of compression, feature analysis, classification, metadata tagging, categorization. The system may handle preprocessing, digital conversion, and post-processing using a variety of methods, ranging from statistical to machine learning. As the data is digitally post-processed, system settings and metadata may be referred to determine how certain logic rules in the application are to operate, which may include mapping certain signal features to certain actions. Based on these mappings, the system operates by sending these post-processed data streams as tokens to the GenAI models and may include saving data locally on the sensory device or another storage device, streaming data to other subsystems or networks.

In the case illustrated in FIG. 23, the user is looking at a display that may include characters, symbols, pictures, colors, videos, live camera footage or other visual, oral or interactive content. In this example, the user is looking at a set of “radial menus” or collection of boxes or circles with data in each one that may be a symbol, character, letter, word or entire phrase. The user has been presented a set of words that surround a central phrase starter word in the middle like a hub and spoke to choose from based on typical functional communication with suggested fringe words and access to predictive keyboard, structured and unstructured language. The user selects options through head motion 2306 and may rapidly compose a phrase by selecting the next desired word presented in the radial menus or adding a new word manually via another input method. The user traverses the interface using head movement gestures, similar to 3-dimensional swipe movements, to compose communication. The user progressively chooses the next word until they're satisfied with the phrase they've composed and may determine how to actuate the phrase. Algorithms may be used to predict the next character, word, or phrase, and may rearrange or alter the expression depending on its intended output including but not limited to appending emoji, symbols, colors, sounds or rearranging to correct for spelling or grammar errors. The user may desire for the phrase to be spoken aloud to a person nearby, thus selecting a “play button” or simply allowing the sentence to time out to be executed automatically. The application creates output based on the user's selected options 4208. If they compose a phrase that is a control command like “turn off the lights”, they may select a “send button” or may, based on semantic natural language processing and understanding, automatically send the phrase to a third party virtual assistant system to execute the command, and turn off the lights. The potential use of metadata, in this example, could simply be geolocation data sourced from other systems such as a geographic information system (GIS) or a global positioning system (GPS) data or WiFi data, or manually personalized geofencing in the application personalization settings, where the system would know if the user is “at home” or “away from home”. On condition that system detects the user is away from home 2310, for example, the metadata may play a role in adapting the language being output to reflect the context of the user. For instance, the system could be configured to speak aloud when at home but send output to a caregiver via text message 2312 and append GPS coordinates when away from home. The system may support collecting and processing historical data from the sensory device, system, subsystems, and output actions to improve the performance and personalization of the system, subsystems, and sensory devices.

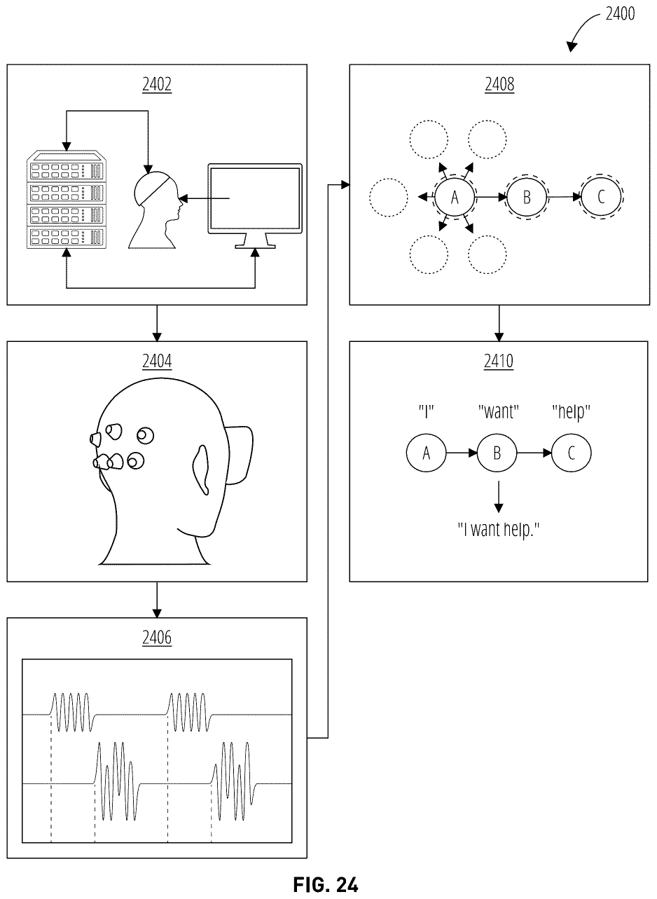

FIG. 24 illustrates a logical diagram of a user wearing an augmented reality headset 2400, in which user wears an EEG-based brain-computer interface headset 2402 containing electrodes that are contacting the scalp 2404. FIG. 24 shows that streaming analog data may be acquired from the brainwave activity 2406. In this manner, the user may be presented a set of words to choose from 2408, compose a phrase, and select what action the system takes using the phrase they've composed 2410.

A user wears an EEG-based brain-computer interface headset 2402 containing electrodes that are contacting the scalp 2404. The electrodes are connected to an amplifier and analog-to-digital processing pipeline. The sensory device (BCI) acquires streaming electrical current data measured in microvolts (μV). The more electrodes connected to the scalp and to the BCI, the more streaming analog data may be acquired from the brainwave activity 2406. The analog streaming data is acquired by the electrodes, pre-processed through amplification, and digitally processed, either directly on the sensory device or via a remotely connected subsystem. The system may include embedded software on the sensory device that handles the pre-processing of the analog signal. The system may include embedded software that handles the digitization and post-processing of the signals. Post-processing may include but not be limited to various models of compression, feature analysis, classification, metadata tagging, categorization. The system may handle preprocessing, digital conversion, and post-processing using a variety of methods, ranging from statistical to machine learning. As the data is digitally post-processed, system settings and metadata may be referred to determine how certain logic rules in the application are to operate, which may include mapping certain signal features to certain actions. Based on these mappings, the system operates by executing commands and may include saving data locally on the sensory device or another storage device, streaming data to other subsystems or networks.

In the case illustrated in FIG. 24, the user is looking at a display that may include characters, symbols, pictures, colors, videos, live camera footage or other visual, oral or interactive content. In this example, the user is looking at a group of concentric circles, arranged in a radial layout, with characters on each circle. The user has been presented a set of words to choose from 2408 based on typical functional communication with suggested fringe words and access to predictive keyboard and may rapidly compose a phrase by selecting the next desired word presented in the outer ring of circles or adding a new word manually. The user progressively chooses the next word until they're satisfied with the phrase they've composed 2410 and may determine how to actuate the phrase. GenAI may be used to predict the next character, word, or phrase, and may rearrange or alter the expression depending on its intended output including but not limited to appending emoji, symbols, colors, sounds or rearranging to correct for spelling or grammar errors. The user may desire for the phrase to be spoken aloud to a person nearby, thus selecting a “play button” or simply allowing the sentence to time out to be executed automatically. If they compose a phrase that is a control command like “turn off the lights”, they may select a “send button” or may, based on semantic natural language processing and understanding, automatically send the phrase to a third party virtual assistant system to execute the command, and turn off the lights. The potential use of metadata, in this example, could simply be geolocation data sourced from other systems such as GIS or GPS data or WiFi data, or manually personalized geofencing in the application personalization settings, where the system may know if the user is “at home” or “away from home”. In this case, the metadata may play a role in adapting the language being output to reflect the context of the user. For instance, the system could be configured to speak aloud when at home but send to a caregiver via text message and append GPS coordinates when away from home. The system may support collecting and processing historical data from the sensory device, system, subsystems, and output actions to improve the performance and personalization of the system, subsystems, and sensory devices.

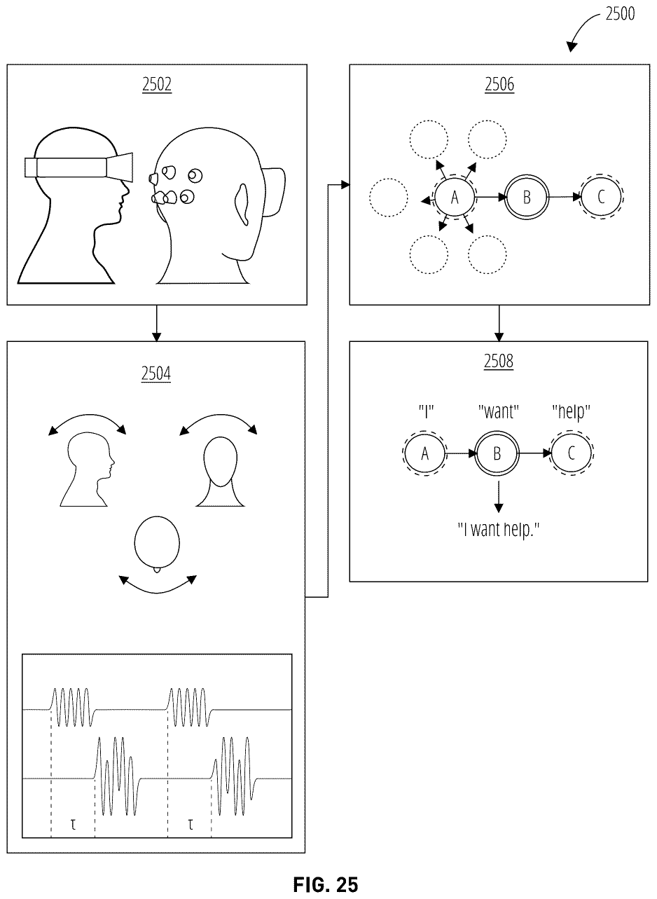

FIG. 25 illustrates a diagram of a use case including a user wearing an augmented reality headset 2500, in which a user wears an augmented reality headset combined with a brain computer interface 2502, having the capabilities described with respect to FIG. 23 and FIG. 24. Both head motion analog input and brainwave activity 2504 may be detected and may allow a user to select from a set of words to choose from 2506, as well as what to do with the phrase they've composed 2508 by selecting those words.

A user is wearing an augmented reality headset combined with a brain computer interface on their head. The headset contains numerous sensors as a combined sensory device including motion and orientation sensors and temporal bioelectric data generated from the brain detected via EEG electrodes contacting the scalp of the user, specifically in the regions where visual, auditory and sensory/touch is processed in the brain. The AR headset may produce visual, auditory or haptic stimulation that is detectible via the brain computer interface, and by processing brainwave data with motion data, the system may provide new kinds of multi-modal capabilities for a user to control the system. The analog streaming data is acquired by the Accelerometer, Gyroscope, Magnetometer and EEG analog-to-digital processor, and digitally processed, either directly on the sensory device or via a remotely connected subsystem. The system may include embedded software on the sensory device that handles the pre-processing of the analog signal. The system may include embedded software that handles the digitization and post-processing of the signals. Post-processing may include but not be limited to various models of compression, feature analysis, classification, metadata tagging, categorization. The system may handle preprocessing, digital conversion, and post-processing using a variety of methods, ranging from statistical to machine learning. As the data is digitally post-processed, system settings and metadata may be referred to determine how certain logic rules in the application are to operate, which may include mapping certain signal features to certain actions. Based on these mappings, the system operates by executing commands and may include saving data locally on the sensory device or another storage device, streaming data to other subsystems or networks.

In the case illustrated in FIG. 25, the user is looking at a display that may include characters, symbols, pictures, colors, videos, live camera footage or other visual, oral or interactive content. In this example, the user is looking at a visual menu system in AR with certain hard to reach elements flickering at different frequencies. The user has been presented a set of items to choose from based on typical functional communication with suggested fringe words and access to predictive keyboard and may rapidly compose a phrase by selecting the next desired word presented in the AR head mounted display or adding a new word manually. Enabling the user affordances of extra-sensory reach of visible objects out of reach within the comfortable range of motion of neck movement. The user progressively chooses the next word until they're satisfied with the phrase they've composed and may determine how to actuate the phrase. Algorithms may be used to predict the next character, word, or phrase, and may rearrange or alter the expression depending on its intended output including but not limited to appending emoji, symbols, colors, sounds or rearranging to correct for spelling or grammar errors. The user may desire for the phrase to be spoken aloud to a person nearby, thus selecting a “play button” or simply allowing the sentence to time out to be executed automatically. If they compose a phrase that is a control command like “turn off the lights”, they may select a “send button” or may, based on semantic natural language processing and understanding, automatically send the phrase to a third party virtual assistant system to execute the command, and turn off the lights. The potential use of metadata, in this example, could simply be geolocation data sourced from other systems such as GIS or GPS data or WIFI data, or manually personalized geofencing in the application personalization settings, where the system may know if the user is “at home” or “away from home”. In this case, the metadata may play a role in adapting the language being output to reflect the context of the user. For instance, the system could be configured to speak aloud when at home but send to a caregiver via text message and append GPS coordinates when away from home. The system may support collecting and processing historical data from the sensory device, system, subsystems, and output actions to improve the performance and personalization of the system, subsystems, and sensory devices.

FIG. 26 is a flow diagram 2600 showing a closed loop bio-signal data flow for a nonverbal multi-input and feedback device such as herein. It may be performed by inputs or a computer of the device. The flow diagram 2600 comprises a human user 2602, electrode sensors 2604, a brain computer interface headset and firmware 2606, an augmented reality mobile application 2608, machine learning capture and training 2610 that may be performed in an edge, peer, or cloud device, and an augmented reality headset 2612. The electrode sensors 2604 may capture 2614 data that is sent for analog-to-digital 2616 conversion. The digital signal may be used for intent detection 2618 resulting in an action trigger 2620 to a user interface 2622. The digital data may further be sent to raw data capture 2626 and may be used as training data 2632 for training and data analysis 2634. Training and data analysis 2634 may yield machine learning parameters 2624 which may be fed back for use in intent detection 2618. The user interface 2622 may determine stimulus placement and timing 2628, which may be used in the augmented reality environment 2630 created by the augmented reality mobile application 2608. The stimulus placement and timing 2636 resulting in the augmented reality headset 2612 and may evoke potential stimulus 2638 in the human user 2602. The user interface 2622 may also generate an output and action 2640.

The flow diagram 2600 includes computer stimulates visual, auditory and somatosensory cortex with evoked potentials; signal processing of real time streaming brain response; human controls computer based on mental fixation of stimulation frequencies; and system may determine different output or actions on behalf of the user for input data received via one or more sensors of the device. Flow diagram 2600 may apply to a user wearing any of the nonverbal multi-input and feedback devices and/or sensors herein. As a result of this being closed-loop biofeedback and sensory communication and control system that stimulates the brains senses of sight, sound, and touch and reads specific stimulation time-based frequencies, and tags them with metadata in real-time as the analog data is digitized, the user may rapidly learn how to navigate and interact with the system using their brain directly. This method of reinforcement learning is known in the rapid development process of the brain's pattern recognition abilities and the creation of neural plasticity to develop new neural connections based on stimulation and entrainment. This further allows the system to become a dynamic neural prosthetic extension of their physical and cognitive abilities. The merging of context awareness metadata, vocabulary, and output and action logic into the central application in addition to a universal interface for signal acquisition and data processing is what makes this system extremely special. Essentially, this system helps reduce the time latency between detecting cognitive intention and achieving the associated desired outcome, whether that be pushing a button, saying a word or controlling robots, prosthetics, smart home devices or other digital systems.

FIG. 27 is a flow diagram 2700 showing multimodal, multi-sensory system for communication and control 2702 for a nonverbal multi-input and feedback device such as herein. It may be performed by inputs or a computer of the device. The flow diagram 2700 comprises multimodal, multi-sensory systems for communication and control 2702 that includes wireless neck and head tracking 2704 and wireless brain tracking 2706. The multimodal, multi-sensory system for communication and control 2702 may further comprise central sensors 2708 for EEG, peripheral sensors 2710 such as EMG, EOG, ECG, and others, an analog to digital signal processor 2712 processing data from the central sensors 2708, and an analog to digital signal processor 2714 processing data from the peripheral sensors 2710. The analog to digital subsystem 2716 and sensor service subsystem 2718 manage output from the analog to digital signal processor 2712 and the analog to digital signal processor 2714, respectively. Output from the analog to digital subsystem 2716 may be sent to a storage subsystem 2760.

Outputs from the analog to digital subsystem 2716 and sensor service subsystem 2718 go to a collector subsystem 2720, which also receives a real-time clock 2722. The collector subsystem 2720 communicates with a recognizer 2724 for EEG data and a classifier 2726 for EMG, EOG, and ECG data, and data from other sensing. The collector subsystem 2720 further communicates to a wireless streamer 2728 and a serial streamer 2730 to interface with a miniaturized mobile computing system 2736 and a traditional workstation 2732, respectively. The traditional workstation 2732 and miniaturized mobile computing system 2736 may communicate with a cloud 2734 for storage or processing. The miniaturized mobile computing system 2736 may assist in wireless muscle tracking 2738 (e.g., EMG data) and wireless eye pupil tracking 2740.

A controller subsystem 2742 accepts input from a command queue 2744 which accepts input from a Bluetooth or BT write callback 2750. The BT write callback 2750 may send commands 2746 to a serial read 2748. The controller subsystem 2742 may send output to the controller subsystem 2742 and a peripherals subsystem 2752. The peripherals subsystem 2752 generates audio feedback 2754, haptic feedback 2756, and organic LED or OLED visual feedback 2758 for the user.

The flow diagram 2700 includes synchronizing signals from multiple biosensors including brain, body (see skin colored arm), eye and movement; processing multiple models concurrently for multi-sensory input; and directing and processing biofeedback through peripheral subsystems. Flow diagram 2700 may apply to a user wearing any of the nonverbal multi-input and feedback devices and/or sensors herein.

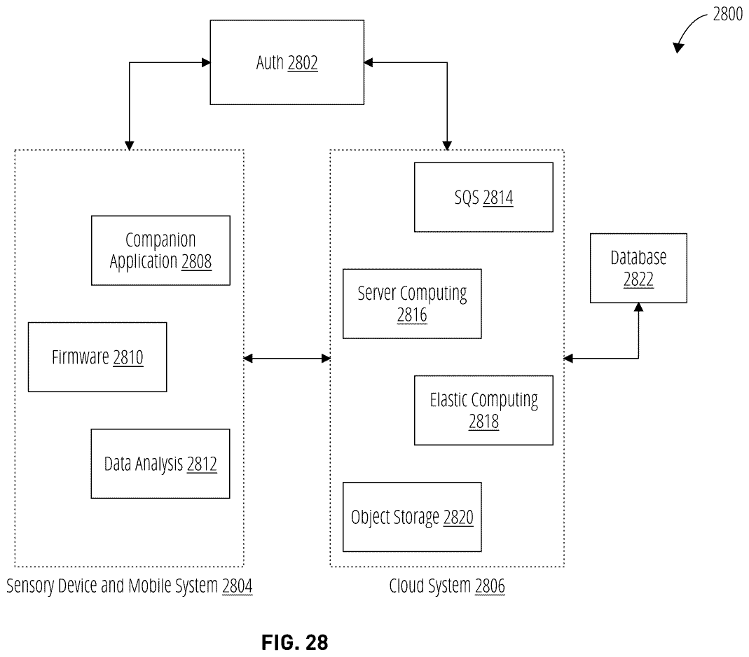

FIG. 28 is a block diagram 2800 showing an example of cloud processing for a nonverbal multi-input and feedback device such as herein. The block diagram 2800 comprises data authentication 2802, a sensory device and mobile system 2804, a cloud system 2806, and a database 2822. The data authentication 2802 module may be configured to authenticate data and communicate with the sensory device and mobile system 2804 and cloud system 2806. The sensory device and mobile system 2804 may include companion application 2808 and data collection, firmware 2810 and data collection, and data analysis 2812 or raw and processed data. The cloud system 2806 may comprise simple queue service or SQS message queuing 2814, server computing 2816 to analyze raw and process data, elastic computing 2818 to build, train, and test machine learning models, and object storage 2820 for persistent storage of biodata, machine learning, and metadata. The database 2822 stores associations and metadata and is in communication with the cloud system 2806.

Block diagram 2800 has the cloud system, the nonverbal multi-input device and an authorization system. Block diagram 2800 includes: machine learning processing signal data on device; metadata enrichment; push raw and processed data to cloud; cloud application building new models for devices; system updates devices remotely and wirelessly; secure and privacy compliant. This configuration is quite powerful but unassumingly simple in this block diagram.

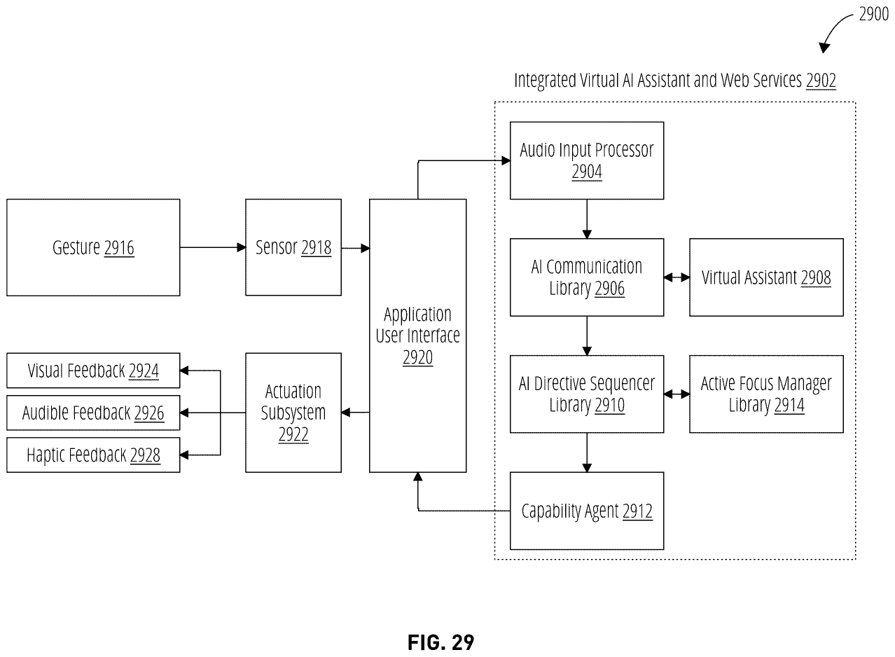

FIG. 29 is a block diagram 2900 showing an example of a system architecture for integrated virtual AI assistant and web services 2902 for a nonverbal multi-input and feedback device such as herein. The block diagram 2900 comprises integrated virtual AI assistant and web services 2902 which may include an audio input processor 2904, an AI communication library 2906, a virtual assistant 2908 such as Alexa, an AI directive sequencer library 2910, a capability agent 2912, and an active focus manager library 2914. A gesture 2916 from a user may be detected by a sensor 2918. An application user interface 2920 may process sensor data and may send data to the audio input processor 2904. The capability agent 2912 may send data back to the application user interface 2920. The application user interface 2920 may signal an actuation subsystem 2922 to provide visual feedback 2924, audible feedback 2926, and haptic feedback 2928.

The block diagram 2900 includes: system manages intention signal acquisition, processing, language composition, and output; in the event where a user wants to send their intention to a virtual assistant (like Alexa, Siri). The blocks outside of the dashed border run on the sensory device, and currently, the blocks inside the dashed line are running in the cloud (e.g., represent a custom configuration for how to use the Alexa service in a cloud architecture.) It may also be possible that all of what is described here as in the cloud may run locally in the sensory device.

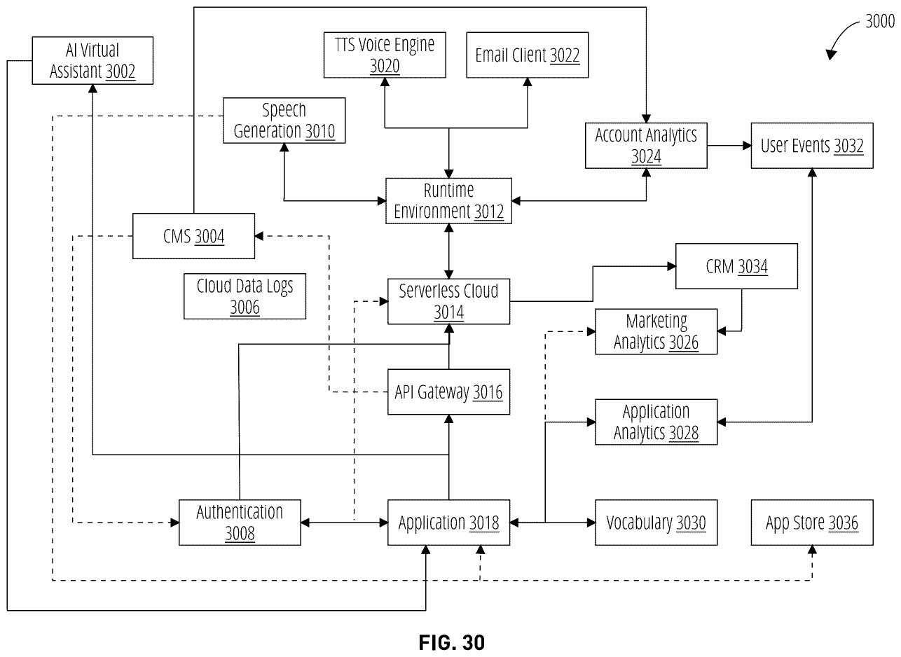

FIG. 30 is a block diagram 3000 showing an example of system operations for a nonverbal multi-input and feedback device such as herein. The block diagram 3000 comprises an AI virtual assistant 3002, such as Alexa, a content management system 3004, cloud data logs 3006, authentication 3008, speech generation 3010, a runtime environment 3012, a serverless cloud 3014, an API gateway 3016, an application 3018, a text-to-speech or TTS voice engine 3020, an email client 3022, account analytics 3024, marketing analytics 3026, application analytics 3028, a vocabulary 3030, user events 3032, a customer relations management 3034, and an app store 3036.

Block diagram 3000 includes: system operation blocks including authentication. This is an example of the complexity of a system operating in the cloud. Everything in this figure is in the cloud, except for the application that is running on the sensory device. The augment/virtual reality application 3018 for the nonverbal multi-input and feedback device may interface with an authentication 3008 module, an API gateway 3016, a vocabulary 3030, application analytics 3028, AI virtual assistant 3002, and marketing analytics 3026. The AI virtual assistant 3002 may communicate back to the application 3018. The application 3018 may also be in direct communication with a serverless cloud 3014 or may communicate with the serverless cloud 3014 through the API gateway 3016. Authentication 3008 may also be in communication with the serverless cloud 3014. The API gateway 3016 further allows the application 3018 to communicate with the content management system 3004, which may be used to store cloud data logs 3006. The content management system 3004 may send data back to the application 3018 through the authentication 3008 module, which may act as a gateway to ensure security and content authorization. Finally, the content management system 3004 may provide data to an account analytics 3024 module. Account analytics 3024 may provide data to a user events 3032 module, which may in turn feed data to application analytics 3028.

The serverless cloud 3014 may allow communication with the runtime environment 3012 and the customer relations management 3034 module. The customer relations management 3034 may provide data for marketing analytics 3026. The runtime environment 3012 may interface with speech generation 3010, a TTS voice engine 3020, an email client 3022, and account analytics 3024. Speech generation 3010 may allow a user to access an app store 3036.

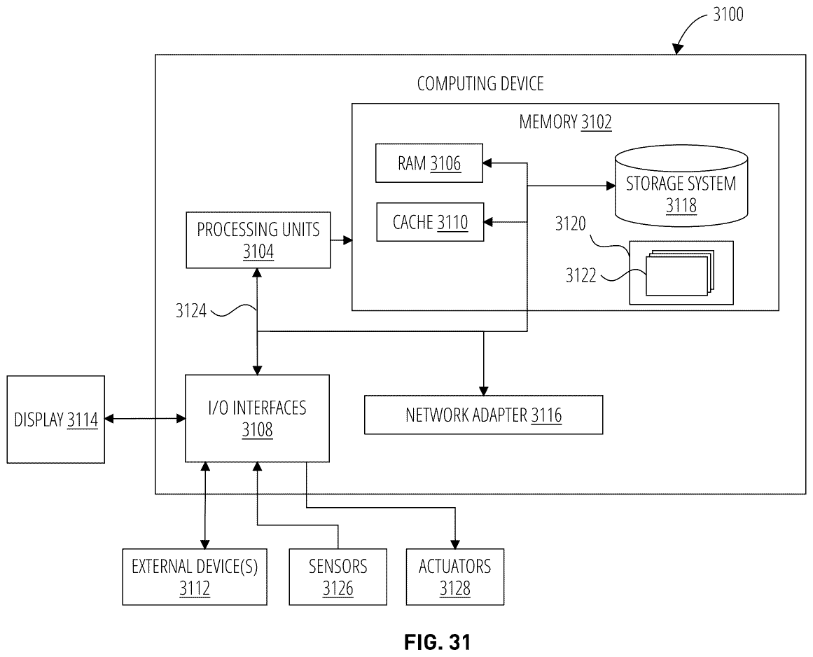

As shown in FIG. 31, a computing device 3100, such as a smart phone or other smart device, is shown in the form of a general-purpose computing device. The components of computing device 3100 may include, but are not limited to, one or more processors or processing units 3104, a system memory 3102, and a bus 3124 that couples various system components including system memory 3102 to processor processing units 3104. Computing device 3100 may include sensors 3126 such as cameras, accelerometers, microphones, etc., and actuators 3128, such as speakers, vibrating or haptic actuators, etc. Computing device 3100 may be a smartphone, a tablet, laptop, desktop computer, or other computing device suitable for implementing the disclosed solution as described herein.

Bus 3124 represents one or more of any of several types of bus structures, including a memory bus or memory controller, a peripheral bus, an accelerated graphics port, and a processor or local bus using any of a variety of bus architectures. By way of example, and not limitation, such architectures include Inter-Integrated Circuit (I2C), Serial Peripheral Interface (SPI), Controller Area Network (CAN), Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computing device 3100 typically includes a variety of computer system readable media. Such media may be any available media that is accessible by computing device 3100, and it includes both volatile and non-volatile media, removable and non-removable media.

System memory 3102 may include computer system readable media in the form of volatile memory, such as Random access memory (RAM) 3106 and/or cache memory 3110. Computing device 3100 may further include other removable/non-removable, volatile/non-volatile computer system storage media. By way of example, a storage system 3118 may be provided for reading from and writing to a non-removable, non-volatile magnetic media (not shown and typically called a “hard drive”). Although not shown, a flash drive, a magnetic disk drive for reading from and writing to a removable, non-volatile magnetic disk (e.g., a “floppy disk”), and an optical disk drive for reading from or writing to a removable, non-volatile optical disk such as a compact disc read only memory (CD-ROM), digital versatile disc read only memory (DVD-ROM), or other optical media, may be provided. In such instances, each may be connected to bus 3124 by one or more data media interfaces. As will be further depicted and described below, system memory 3102 may include at least one program product having a set (e.g., at least one) of program modules that are configured to carry out the functions of the disclosure.

Program/utility 3120 having a set (at least one) of program modules 3122 may be stored in system memory 3102 by way of example, and not limitation, as well as an operating system, one or more application programs, other program modules, and program data. Each of the operating system, one or more application programs, other program modules, and program data or some combination thereof, may include an implementation of a networking environment. Program modules 3122 generally carry out the functions and/or methodologies of the disclosure as described herein.

Computing device 3100 may also communicate with one or more external devices 3112 such as a keyboard, a pointing device, a display 3114, etc. ; one or more devices that allow a user to interact with computing device 3100; and/or any devices (e.g., network card, modem, etc.) that allow computing device 3100 to communicate with one or more other computing devices. Such communication may occur via I/O interfaces 3108. I/O interfaces 3108 may also manage input from computing device 3100 sensors 3126, as well as output to actuators 3128. Still yet, computing device 3100 may communicate with one or more networks such as a local area network (LAN), a general wide area network (WAN), and/or a public network (e.g., the Internet) via network adapter 3116. As depicted, network adapter 3116 communicates with the other components of computing device 3100 via bus 3124. It may be understood that although not shown, other hardware and/or software components could be used in conjunction with computing device 3100. Examples include, but are not limited to, microcode, device drivers, redundant processing units, external disk drive arrays, redundant array of independent disks (RAID) systems, tape drives, data archival storage systems, etc.

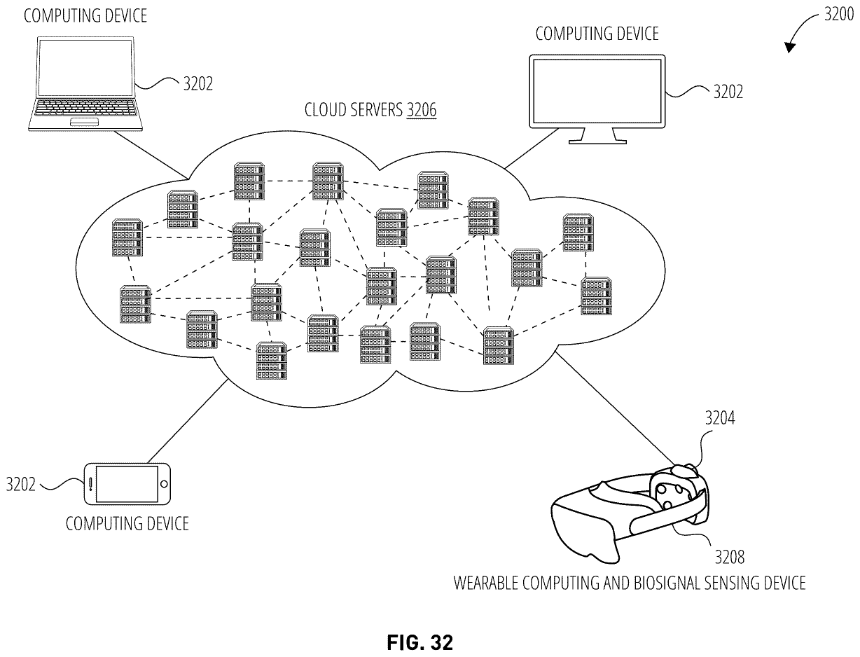

Referring now to FIG. 32, an illustrative cloud computing system 3200 is depicted. “Cloud computing” refers to a model for enabling convenient, on-demand network access to a shared pool of configurable computing resources (e.g., networks, servers, storage, applications, and services) that may be rapidly provisioned and released with minimal management effort or service provider interaction. This cloud model promotes availability and is comprised of at least five characteristics, at least three service models, and at least four deployment models. Examples of commercially hosted cloud computing systems 3200 include Amazon Web Services (AWS), Google Cloud, Microsoft Azure, etc.

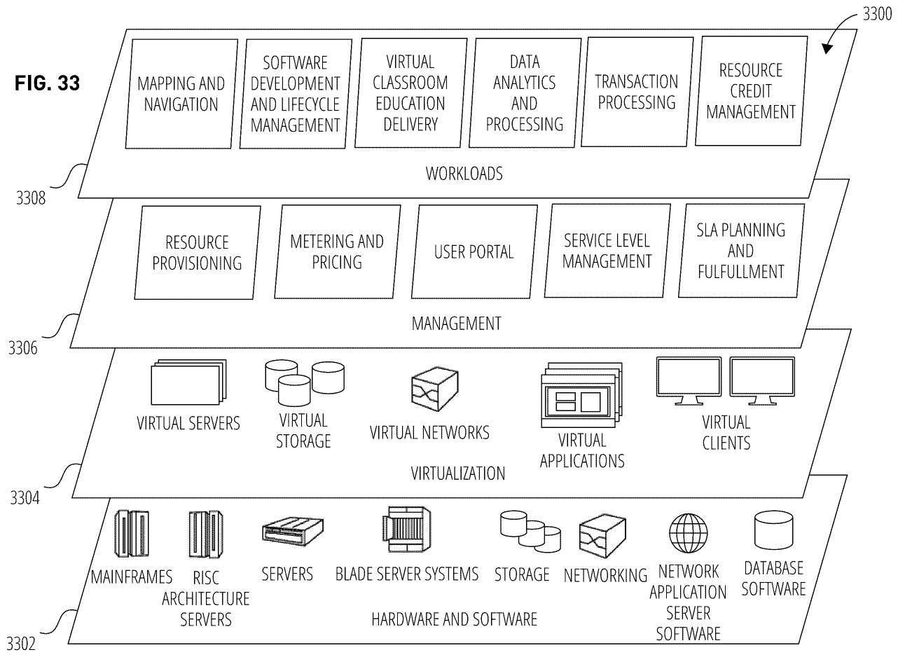

As shown, cloud computing system 3200 may comprise one or more cloud servers 3206, with which computing devices 3202 such as, for example, personal digital assistants (PDAs), smart devices such as smart phones, laptops, desktop computers, and/or wearable computing and biosignal sensing device 3208 brain computer interfaces or BCIs 3204 may communicate. This allows for infrastructure, platforms, and/or software to be offered as services (as described above in FIG. 33) from cloud servers 3206, so as to not require each client to separately maintain such resources. It is understood that the types of computing devices shown in FIG. 32 are intended to be illustrative and not limiting, and that cloud servers 3206 may communicate with any type of computerized device over any type of network and/or network/addressable connection (e.g., using a web browser).

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that may be rapidly provisioned and released with minimal management effort or interaction with a provider of the service. This cloud model may include at least five characteristics, at least three service models, and at least four deployment models.

Characteristics are as follows:
On-demand self-service: a cloud consumer may unilaterally provision computing capabilities, such as server time and network storage, as needed automatically without requiring human interaction with the service's provider.
Broad network access: capabilities are available over a network and accessed through standard mechanisms that promote use by heterogeneous thin or thick client platforms (e.g., mobile phones, laptops, and PDAs).
Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).
Rapid elasticity: capabilities may be rapidly and elastically provisioned, in some cases automatically, to quickly scale out and rapidly released to quickly scale in. To the consumer, the capabilities available for provisioning often appear to be unlimited and may be purchased in any quantity at any time.
Measured service: cloud systems automatically control and optimize resource use by leveraging a metering capability at some level of abstraction appropriate to the type of service (e.g., storage, processing, bandwidth, and active user accounts). Resource usage may be monitored, controlled, and reported, providing transparency for both the provider and consumer of the utilized service.

Service Models are as follows:
Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.
Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.
Infrastructure as a Service (IaaS): the capability provided to the consumer is to provision processing, storage, networks, and other fundamental computing resources where the consumer is able to deploy and run arbitrary software, which may include operating systems and applications. The consumer does not manage or control the underlying cloud infrastructure but has control over operating systems, storage, deployed applications, and possibly limited control of select networking components (e.g., host firewalls).

Deployment Models are as follows:
Private cloud: the cloud infrastructure is operated solely for an organization. It may be managed by the organization or a third party and may exist on-premises or off-premises.
Community cloud: the cloud infrastructure is shared by several organizations and supports a specific community that has shared concerns (e.g., mission, security needs, policy, and compliance considerations). It may be managed by the organizations or a third party and may exist on-premises or off-premises.
Public cloud: the cloud infrastructure is made available to the general public or a large industry group and is owned by an organization selling cloud services.
Hybrid cloud: the cloud infrastructure is a composition of two or more clouds (private, community, or public) that remain unique entities but are bound together by standardized or proprietary technology that supports data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 33, a set of cloud computing functional abstraction layers 3300 provided by cloud computing systems 3200 such as those illustrated in FIG. 32 is shown. It may be understood in advance that the components, layers, and functions shown in FIG. 33 are intended to be illustrative, and the disclosure is not limited thereto. As depicted, the following layers and corresponding functions are provided:

Hardware and software layer 3302 includes hardware and software components. Examples of hardware components include mainframes, reduced instruction set computer (RISC) architecture based servers, servers, blade servers, storage devices, and networks and networking components. Examples of software components include network application server software and database software.

Virtualization layer 3304 provides an abstraction layer from which the following exemplary virtual entities may be provided: virtual servers; virtual storage; virtual networks, including virtual private networks; virtual applications; and virtual clients.

Management layer 3306 provides the exemplary functions described below. Resource provisioning provides dynamic procurement of computing resources and other resources that are utilized to perform tasks within the Cloud computing environment. Metering and Pricing provide cost tracking as resources are utilized within the cloud computing environment, and billing or invoicing for consumption of these resources. In one example, these resources may comprise application software licenses. Security provides identity verification for users and tasks, as well as protection for data and other resources. The user portal provides access to the Cloud computing environment for both users and system administrators. Service level management provides Cloud computing resource allocation and management such that the service levels needed are met. Service Level Agreement (SLA) planning and fulfillment provide pre-arrangement for, and procurement of, Cloud computing resources for which a future need is anticipated in accordance with an SLA.

Workloads layer 3308 provides functionality for which the cloud computing environment is utilized. Examples of workloads and functions which may be provided from this layer include mapping and navigation; software development and lifecycle management; virtual classroom education delivery; data analytics processing; transaction processing; and resource credit management. As mentioned above, all of the foregoing examples described with respect to FIG. 33 are illustrative, and the disclosure is not limited to these examples.

### LISTING OF DRAWING ELEMENTS

100 exemplary head-mounted biosignal acquisition platform
102 sensor module
104 wired coupler
200 modular head-mounted biosignal acquisition system
202 tensioning element
300 structural chassis assembly
302 structural chassis
304 front optical lens assembly
306 strap assembly connector arm
308 optical see-through lens
310 eye tracking sensor insert
312 prescription lens insert
314 face sensor insert
316 rotational dial
318 electrical contact
320 peripheral connector
322 auditory stimulator
324 camera
326 lidar sensor
328 projector
330 microphone
332 notch
334 face pad
336 lower portion
338 near-field eye tracking sensor
340 near-field eye tracking emitter
400 wearable biosignal acquisition strap assembly
402 top strap assembly
404 rear strap assembly
406 top strap frame
408 top strap sensing assembly
410 rear strap frame
412 first rear strap sensing assembly
414 second rear strap sensing assembly
416 tensioning interface
500 standardized electromechanical mating interface
502 first interface member
504 second interface member
506 first magnet
508 first electrical contact
510 second magnet
512 second electrical contact
514 biosignal
516 ground
518 power
520 power conductor
522 ground conductor
524 biosignal channel conductor
526 digital identification conductor
528 signal channel conductor
600 tensioning element installation and operation
602 tensioning key
604 traverse
606 rolling or rotational motion
608 tension and fit adjustment
700 strap assembly connector arm details
702 track or slot
704 depth-offset conductive channel
706 concentric ring contact
800 processing system
802 hip-pack accessory
804 touch display
806 isolated trigger input
808 general purpose analog and/or digital interface
900 exemplary modular biosignal acquisition platform components
902 USB-C cable
904 Lightning cable
906 USB-A cable
908 US power adapter
910 EU power adapter
1000 routine
1002 block
1004 block
1006 block
1008 block
1010 block
1012 block
1014 block
1100 user agency and capability augmentation system
1102 user
1104 wearable computing and biosignal sensing device
1106 biosignals
1108 background material
1110 sensor data
1112 other device data
1114 application context
1116 prompt composer
1118 GenAI
1120 multimodal output stage
1122 output modalities
1124 utterance
1126 written text
1128 multimodal artifact
1130 other user agency
1132 encoder/parser
1134 non-language user agency device
1136 biosignals prompt
1138 context prompt
1140 user input prompt
1142 shared data
1144 prompt
1146 multimodal output
1148 output mode selection signal
1200 biosignals subsystem
1202 additional biosensors
1204 biosignals classifier
1206 EEG tokenizer
1208 kinematic tokenizer
1210 additional tokenizers
1300 context subsystem
1302 raw background material tokenizer
1304 final background material tokenizer
1306 raw sensor data tokenizer
1308 final sensor data tokenizer
1310 raw device data tokenizer
1312 final device data tokenizer
1314 raw application context tokenizer
1316 final application context tokenizer
1318 context prompt composer
1320 preliminary tokens
1322 final tokens
1400 routine
1402 block
1404 block
1406 block
1408 block
1410 block
1412 block
1414 block
1416 decision block
1418 block
1420 block
1422 block
1500 multiuser system
1502a simultaneous user
1502b simultaneous user
1502c simultaneous user
1502d simultaneous user
1504 network
1600 exemplary tokenizer
1602 input data
1604 tokenizable element
1606 token
1608 tokenized output
1700 BCI headset system
1702 augmented reality display lens
1704 top cover
1706 adjustable strap
1708 padding
1710 ground/reference electrode
1712 ground/reference electrode adjustment dial
1714 biosensor electrodes
1716 battery cell
1718 fit adjustment dial
1720 control panel cover
1722 control panel
1724 biosensor electrode adjustment dials
1726 auxiliary electrode ports
1728 power switch
1730 USB port
1732 forward housing
1734 smart phone
1736 smart phone slot
1800 BCI +AR environment
1802 human user
1804 sensor
1806 EEG analog to digital converter
1808 Audio/Video/Haptic Output
1810 processing
1812 augmented reality glasses
1814 strap
1816 BCI
1900 augmented reality device logic
1902 processing units
1904 CPU
1906 GPU
1908 NPU
1910 WiFi
1912 Bluetooth
1914 speakers
1916 microphones
1918 memory
1920 logic
1922 graphics engine
1924 camera
1926 visual display
1928 vibration/haptic driver
1930 cloud
1932 Bluetooth keyboard
2000 block diagram of nonverbal multi-input and feedback device
2002 application
2004 multimodal input and intent detection
2006 context awareness
2008 GenAI
2010 output and action
2012 touch
2014 bio-signals
2016 keyboard
2018 facial tracking
2020 eye and pupil tracking
2022 alternative inputs
2024 biometrics
2026 environment
2028 object recognition
2030 facial recognition
2032 voice recognition
2034 date and time
2036 history
2038 location
2040 proximity
2042 other metadata
2044 flat screen display
2046 augmented/virtual reality
2048 virtual AI assistant
2050 synthesized voice
2052 prosthetic device
2054 social media and messaging
2056 Media Consumption
2100 block diagram of a single framework of a nonverbal multi-input and feedback device
2102a sensor
2102b sensor
2102c sensor
2102d sensor
2102e sensor
2102f sensor
2104 input gesture
2106 context awareness
2108 machine learning
2110 output expression
2112 destination
2114 touch
2116 movement
2118 mental
2120 glances
2122 audible
2124 breath
2126 time synchronization
2128 configure data sources
2130 configure data processing parameters
2132 configure timing
2134 metadata tagging
2136 acquire analog data streams
2138 convert to digital data streams
2140 analyze data streams
2142 execute digital operations for actuation
2144 text
2146 symbol
2148 color
2150 image
2152 sound
2154 vibration
2156 mobile
2158 wearable 1
2160 wearable 2
2162 implant 1
2164 implant 2
2166 prosthetic 1
2200 block diagram of nonverbal multi-input and feedback device
2202 analog input
2204 sensors
2206 processing
2208 digital output
2210 output methods
2212 historical data
2214 learning system
2216 network
2218 personalization settings
2220 metadata
2222 application
2224 primary and secondary languages
2226 visual
2228 audible
2230 Written
2300 logical diagram of a user wearing an augmented reality headset
2302 head motion analog input
2304 headset with head motion detection sensors
2306 user selects options through head motion
2308 application creates output based on the user's selected options
2310 condition that system detects the user is away from home
2312 send output to a caregiver via text message
2400 logical diagram of a user wearing an augmented reality headset
2402 user wears an EEG-based brain-computer interface headset
2404 electrodes that are contacting the scalp
2406 streaming analog data may be acquired from the brainwave activity
2408 set of words to choose from
2410 Phrase They've Composed
2500 diagram of a use case including a user wearing an augmented reality headset
2502 augmented reality headset combined with a brain computer interface
2504 head motion analog input and brainwave activity
2506 set of words to choose from
2508 phrase they've composed
2600 flow diagram
2602 human user
2604 electrode sensors
2606 brain computer interface headset and firmware
2608 augmented reality mobile application
2610 machine learning capture and training
2612 augmented reality headset
2614 capture
2616 analog-to-digital
2618 intent detection
2620 action trigger
2622 user interface
2624 machine learning parameters
2626 raw data capture
2628 stimulus placement and timing
2630 augmented reality environment
2632 training data
2634 training and data analysis
2636 stimulus placement and timing
2638 evoke potential stimulus
2640 output and action
2700 Flow Diagram
2702 multimodal, multi-sensory system for communication and control
2704 wireless neck and head tracking
2706 wireless brain tracking
2708 central sensors
2710 peripheral sensors
2712 analog to digital signal processor
2714 analog to digital signal processor
2716 analog to digital subsystem
2718 sensor service subsystem
2720 collector subsystem
2722 real-time clock
2724 recognizer
2726 classifier
2728 wireless streamer
2730 serial streamer
2732 traditional workstation
2734 cloud
2736 miniaturized mobile computing system
2738 wireless muscle tracking
2740 wireless eye pupil tracking
2742 controller subsystem
2744 command queue
2746 command
2748 serial read
2750 BT write callback
2752 peripherals subsystem
2754 audio feedback
2756 haptic feedback
2758 OLED visual feedback
2760 storage subsystem
2800 block diagram
2802 data authentication
2804 sensory device and mobile system
2806 cloud system
2808 companion application
2810 firmware
2812 data analysis
2814 SQS message queuing
2816 server computing
2818 elastic computing
2820 object storage
2822 database
2900 block diagram
2902 integrated virtual AI assistant and web services
2904 audio input processor
2906 AI communication library
2908 virtual assistant
2910 AI directive sequencer library
2912 capability agent
2914 active focus manager library
2916 gesture
2918 sensor
2920 application user interface
2922 actuation subsystem
2924 visual feedback
2926 audible feedback
2928 haptic feedback
3000 block diagram
3002 AI virtual assistant
3004 content management system
3006 cloud data logs
3008 authentication
3010 speech generation
3012 runtime environment
3014 serverless cloud
3016 API gateway
3018 application
3020 TTS voice engine
3022 email client
3024 account analytics
3026 marketing analytics
3028 application analytics
3030 vocabulary
3032 user events
3034 customer relations management
3036 app store
3100 computing device
3102 system memory
3104 processing units
3106 Random access memory (RAM)
3108 I/O interfaces
3110 cache memory
3112 external devices
3114 display
3116 network adapter
3118 storage system
3120 program/utility
3122 program modules
3124 bus
3126 sensors
3128 actuators
3200 cloud computing system
3202 computing device
3204 BCI
3206 cloud servers
3208 wearable computing and biosignal sensing device
3300 cloud computing functional abstraction layers
3302 hardware and software layer
3304 virtualization layer
3306 management layer
3308 Workloads Layer

Various functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

Within this disclosure, different entities (which may variously be referred to as “units,” “circuits,” other components, etc.) may be described or claimed as “configured” to perform one or more tasks or operations. This formulation—[entity] configured to [perform one or more tasks]—is used herein to refer to structure (i.e., something physical, such as an electronic circuit). More specifically, this formulation is used to indicate that this structure is arranged to perform the one or more tasks during operation. A structure may be said to be “configured to” perform some task even if the structure is not currently being operated. A “credit distribution circuit configured to distribute credits to a plurality of processor cores” is intended to cover, for example, an integrated circuit that has Circuitry that performs this function during operation, even if the integrated circuit in question is not currently being used (e.g., a power supply is not connected to it). Thus, an entity described or recited as “configured to” perform some task refers to something physical, such as a device, circuit, memory storing program instructions executable to implement the task, etc. This phrase is not used herein to refer to something intangible.

The term “configured to” is not intended to mean “configurable to.” An unprogrammed field programmable gate array (FPGA), for example, would not be considered to be “configured to” perform some specific function, although it may be “configurable to” perform that function after programming.

Reciting in the appended claims that a structure is “configured to” perform one or more tasks is expressly intended not to invoke 35 U.S.C. § 112(f) for that claim element. Accordingly, claims in this application that do not otherwise include the “means for” [performing a function] construct should not be interpreted under 35 U.S.C § 112(f).

As used herein, the term “based on” is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

As used herein, the phrase “in response to” describes one or more factors that trigger an effect. This phrase does not foreclose the possibility that additional factors may affect or otherwise trigger the effect. That is, an effect may be solely in response to those factors or may be in response to the specified factors as well as other, unspecified factors. Consider the phrase “perform A in response to B.” This phrase specifies that B is a factor that triggers the performance of A. This phrase does not foreclose that performing A may also be in response to some other factor, such as C. This phrase is also intended to cover an embodiment in which A is performed solely in response to B.

As used herein, the terms “first,” “second,” etc. are used as labels for nouns that they precede and do not imply any type of ordering (e.g., spatial, temporal, logical, etc.), unless stated otherwise. For example, in a register file having eight registers, the terms “first register” and “second register” may be used to refer to any two of the eight registers, and not, for example, just logical registers 0 and 1.

When used in the claims, the term “or” is used as an inclusive or and not as an exclusive or. For example, the phrase “at least one of x, y, or z” means any one of x, y, and z, as well as any combination thereof.

Having thus described illustrative embodiments in detail, it will be apparent that modifications and variations are possible without departing from the scope of the disclosure as claimed. The scope of disclosed subject matter is not limited to the depicted embodiments but is rather set forth in the following Claims.

Terms used herein may be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, systems, methods and media for carrying out the several purposes of the disclosed subject matter. It is important, therefore, that the claims be regarded as including such equivalent constructions insofar as they do not depart from the spirit and scope of the disclosed subject matter.

What is claimed is:

1. A head-mounted biosignal acquisition platform comprising:
a structural chassis configured to be worn on a user's head;
a standardized electromechanical mating interface disposed on the structural chassis, the standardized electromechanical mating interface defining:
a physical connector geometry;
a mechanical retention specification;
an electrical pin assignment comprising:
at least one power conductor, at least one ground conductor, at least one biosignal channel conductor, and at least one digital identification conductor; and
a module-identification signaling protocol; and
at least one sensor module coupled to the structural chassis via the standardized electromechanical mating interface; and
wherein the at least one sensor module is configured to acquire a biosignal comprising at least one of an electroencephalography signal, a functional near-infrared spectroscopy signal, an ultrasound signal, an electromyography signal, and an electrical stimulation signal;
wherein the standardized interface is defined by the physical connector geometry, mechanical retention specification, electrical pin assignment, and module-identification signaling protocol such that the structural chassis is configured to physically and electrically engage any sensor module conforming to all of the physical connector geometry, mechanical retention specification, electrical pin assignment, and module-identification signaling protocol.

2. The platform of claim 1, wherein the standardized electromechanical mating interface comprises at least one magnet and at least one electrical contact; and wherein the at least one magnet provides mechanical retention force between the structural chassis and the at least one sensor module and the at least one electrical contact simultaneously provides at least one of the at least one power conductor, the at least one ground conductor, the at least one biosignal channel conductor, and the at least one digital identification conductor when the at least one sensor module is mechanically coupled to the structural chassis.

3. The platform of claim 2, wherein the at least one electrical contact comprises at least one of concentric ring contacts, pogo-pin arrays, and flex-circuit interposers, and wherein each is mated with at least one of a permanent magnet and an electropermanent magnet.

4. The platform of claim 1, further comprising a non-sensing comfort insert dimensionally compatible with the standardized electromechanical mating interface and configured to couple to the structural chassis in place of the at least one sensor module;
wherein the at least one sensor module is configured for independent sterilization from the structural chassis;
wherein the at least one sensor module may be implemented as a single-use disposable insert; and
wherein the structural chassis is operable with at least one of the at least one sensor module and the non-sensing comfort insert installed at the standardized interface.

5. The platform of claim 1, wherein the at least one sensor module comprises at least one of an interchangeable sensor cartridge including dry or wet electroencephalography electrode arrays, a functional near-infrared spectroscopy emitter and detector pairs, transcranial ultrasound transducer arrays, transcranial electrical stimulation electrodes, transcranial magnetic stimulation coil elements, and a bridging module configured to communicate with an implantable biosignal acquisition device.

6. The platform of claim 5, wherein the platform is configured to alternate between acquisition and stimulation modes on a same standardized electromechanical mating interface.

7. The platform of claim 1, further comprising an external body-worn compute module connected to the structural chassis via a cable or a wireless link, wherein the external body-worn compute module provides computational resources for machine learning inference.

8. The platform of claim 7, wherein the external body-worn compute module exposes the standardized electromechanical mating interface to third-party peripherals including at least one of keyboards, joystick controllers, expansion sensor arrays, and machine learning accelerator modules.

9. The platform of claim 1, wherein the structural chassis incorporates a processing system internal to the structural chassis, the processing system being configured to perform signal processing of biosignals acquired by the at least one sensor module, and wherein the platform is operable without an external body-worn compute module.

10. A method of operating a modular biosignal acquisition platform, comprising:
detecting, by a processing system coupled to a structural chassis of a head-mounted device, one or more sensor modules connected to the structural chassis via a standardized electromechanical mating interface;
identifying, from a module-identification signal received via the standardized interface, a module type and a capability profile for each detected sensor module;
constructing, by the processing system, a sensor configuration representation based on the identified sensor modules;
computing, for each channel of each detected sensor module, a confidence measure based on at least one of electrical impedance, signal variance, power-line interference, and contact-quality metric; and
adjusting, by the processing system, a weight assigned to each channel in a signal-processing pipeline based on the computed confidence measure.

11. The method of claim 10, wherein detecting the one or more sensor modules and constructing the sensor configuration representation occur automatically upon power-on of the platform or upon connection or removal of the one or more sensor modules.

12. The method of claim 10, further comprising alerting a user via at least one of a visual augmented reality overlay, an audio tone, and a haptic cue to reposition or replace the one or more sensor modules that have a low computed confidence measure.

13. The method of claim 10, wherein the module-identification signal further comprises calibration metadata, and wherein identifying the module type and the capability profile further comprises reading the calibration metadata.

14. The method of claim 10, wherein computing the confidence measure comprises analyzing contact-quality proxies associated with each channel of each detected sensor module.

15. The method of claim 10, wherein adjusting the weight comprises reducing weighting in a signal-processing model for channels with a low computed confidence measure.

16. A wearable biosignal acquisition strap assembly comprising:
a first strap segment and a second strap segment configured to be worn on a user's head;
at least one tensioning element disposed along at least one of the first strap segment or the second strap segment, the at least one tensioning element configured to apply mechanical tension to maintain a fit of the strap assembly on the user's head; and
at least one electrical conductor integrated with the at least one tensioning element, wherein the at least one tensioning element and the at least one electrical conductor together form a composite structure that, during operation of the strap assembly, concurrently applies the mechanical tension and conducts at least one of a biosignal and a power signal between a sensor module and a processing system.

17. The strap assembly of claim 16, further comprising an adjustment mechanism coupled to the first strap segment and the second strap segment and configured to adjust the mechanical tension applied by the at least one tensioning element via a rolling or rotational motion;
wherein the adjustment mechanism comprises a plurality of electrical conductors disposed within or integrated into the adjustment mechanism such that the adjustment mechanism forms a continuous electrical signal pathway between the first strap segment and the second strap segment through the adjustment mechanism across a range of rotational positions of the adjustment mechanism.

18. The strap assembly of claim 17, wherein the adjustment mechanism comprises a rotational dial containing at least one of concentric conductive rings and brush contacts configured to maintain electrical continuity across rotational positions, allowing multi-channel biosignal data to pass through the dial.

19. The strap assembly of claim 16, wherein the at least one tensioning element comprises at least one of conductive fibers woven or braided around a tension cable, a conductive stainless-steel wire rope with an insulating coating, co-extruded composites, and a flex-circuit ribbon integrated with a tensioning tape.

20. The strap assembly of claim 16, wherein the at least one electrical conductor provides shielding against electromagnetic interference to maintain biosignal fidelity.

21. The strap assembly of claim 17, wherein the adjustment mechanism utilizes press-in or pull-out secondary motions to engage independent adjustment modes, and wherein the adjustment mechanism includes ratcheted detents to provide discrete size steps and a lock feature to secure the adjustment mechanism at a selected position.

22. An accessory interface specification for a head-mounted device, comprising:
a physical connector geometry;
a mechanical retention specification;
an electrical pin assignment defining at least one power conductor, at least one ground conductor, at least one biosignal channel conductor, and at least one digital identification conductor; and
a module-identification signaling protocol;
wherein the accessory interface specification is configured such that any module conforming to the physical connector geometry, mechanical retention specification, electrical pin assignment, and module-identification signaling protocol is physically and electrically engageable with the head-mounted device implementing the accessory interface specification without modification of the head-mounted device; and
wherein the module-identification signaling protocol is configured to identify a module type and a capability profile of the conforming module to a processing system of the head-mounted device.

23. A non-transitory computer-readable medium storing instructions that, when executed by a processing system coupled to a structural chassis of a head-mounted device, cause the processing system to:
detect one or more sensor modules connected to the structural chassis via a standardized electromechanical mating interface;
identify, from a module-identification signal received via the standardized interface, a module type and a capability profile for each detected sensor module;
construct a sensor configuration representation based on the identified sensor modules;
compute, for each channel of each detected sensor module, a confidence measure based on at least one of electrical impedance, signal variance, power-line interference, and contact-quality metric; and
adjust a weight assigned to each channel in a signal-processing pipeline based on the computed confidence measure, thereby improving signal-to-noise ratio of biosignals acquired by the head-mounted device.
